(19) United States
(12) Patent Application Publication
(10) Pub. No.: US 2026/0367087 A1
(43) Pub. Date: Nov. 19, 2026

(54) METHODS AND SYSTEM FOR DETECTING EXOSOMAL THOMSEN-FRIENDENREICH ANTIGEN EXPRESSION AND ITS USE IN CANCER IMMUNOTHERAPY

(71) Applicant: Roswell Park Cancer Institute Corporation, Buffalo, NY (US)

(72) Inventors: Grace K. Dy, Buffalo, NY (US); Yun Wu, Buffalo, NY (US); Kate W. Rittenhouse-Olson, Buffalo, NY (US)

(21) Appl. No.: 19/639,706

(22) Filed: Apr. 6, 2026

Related U.S. Application Data
(60) Provisional application No. 63/787,420, filed on Apr. 11, 2025.

(51) Int. Cl. G01N 33/575; G01N 33/543

(52) U.S. Cl. CPC G01N 33/5759; G01N 33/543; G01N 2333/70596; G01N 2800/52

(57) ABSTRACT

Disclosed are methods and systems to detect expression level of tumor-derived exosomal (TEX) biomarkers in liquid biopsy samples and assess the efficacy of a cancer immunotherapy response in a subject with cancer.

### CROSS-REFERENCE TO RELATED APPLICATIONS

This application claims the benefit of priority to U.S. Provisional Application No. 63/787,420, filed Apr. 11, 2025, which is incorporated by reference herein in its entirety.

### STATEMENT REGARDING FEDERALLY SPONSORED RESEARCH

This invention was made with Government Support under Grant Nos. R21CA267934 and R01CA272827 awarded by the National Cancer Institute part of the National Institutes of Health. The Government has certain rights in the invention.

### BACKGROUND

Exosomes are small (30-200 nm) membrane-bound vesicles that are released into the extracellular milieu. Exosomes contain growth factors, proteins, signaling lipids, and various types of RNA inducing messenger RNA (mRNA) and microRNA (miRNA). Their protein and RNA contents mediate many, if not most, of the effects on the cells with which the exosomes communicate.

Immune checkpoint inhibitors (ICIs) (e.g. pembrolizumab and nivolumab) have revolutionized the treatment for cancer. Unfortunately, only a subset of patients develops durable clinical benefits and there is no reliable method to predict which patients will show clinical benefit. Currently, imaging modalities are used to monitor the response to ICI therapy. However, imaging is limited by issues such as pseudo progression and delayed response with ICI therapy. Tissue-based assays are used to select patients and predict response to ICI therapy. However, there are several disadvantages to tissue-based assays. First, tumor PD-L1 expression has a disappointing 28.9% accuracy in predicting response to ICI therapy. Second, baseline tissue specimens are often insufficient for testing in routine practice, such as in 20-30% of lung cancer patients. Third, analysis of immune signatures in longitudinal tumor samples would be ideal to monitor treatment response, however, repeated invasive biopsies to evaluate dynamic changes in antigen expression are not feasible in solid malignancies. There is a critical need to address imaging and tissue-based limitations, effectively predict and monitor response to ICI therapy, and improve patient care and clinical outcomes.

Liquid biopsy detects tumor-derived biomarkers in body fluids such as blood, allows sequential monitoring of cancer development, complements medical imaging and risk factor data, provides a more comprehensive portrait of cancer, and thus represents a new and non-invasive strategy to address this unmet need. However, current cancer liquid biopsy biomarkers, such as CEA and CA-19, lack sensitivity and specificity. While characterization of circulating tumor DNA (ctDNA) can be more specific, this is costly and still of relatively low sensitivity. In addition, some test platforms require the availability of matching tissue specimens for the initial testing (variable from assay to assay, many patients especially lung cancer patients do not have sufficient tissue available for testing). Depending on the ctDNA platform as well, mutations arising from clonal hematopoietic cells may not be distinguished and affect interpretation. Moreover, currently the turnaround time of ctDNA still takes at least 1-2 weeks to process. For all these reasons, the ctDNA liquid biopsy assay is not routinely used for response monitoring. Due to these assay limitations, minimal residual disease assessment for solid tumors such as nonsmall cell lung cancer (NSCLC) remains an unmet need to help stratify patients according to risk groups to both determine need for adjuvant therapy such as immunotherapy and to predict its success. Therefore, there is a need for a non-invasive companion diagnostic platform for cancer-specific assessment of biomarkers expressed on the cell surface.

### SUMMARY

Disclosed herein are methods and systems to detect expression level of tumor-derived exosomal (TEX) biomarkers in liquid biopsy samples and assess the efficacy of a cancer immunotherapy response in a subject with cancer. In some aspects, disclosed herein is a two-step procedure using JAA-F11 antibody to the TF-Ag-α (alpha-linked Thomsen-Friendenreich glycoantigen; Galβ1-3GalNAc alpha) to capture tumor-associated exosomes and antibody to a variety of different antigens that will indicate whether a certain treatment such as immunotherapy will be successful.

In one aspect, disclosed herein are methods of detecting a tumor-derived exosomes (TEX) biomarker (including, but not limited to exosomes derived from lung cancer, breast cancer, prostate cancer, colon cancer, ovarian cancer, or bladder cancer) in a subject using liquid biopsy (including, but not limited to cerebrospinal fluid, saliva, amniotic fluid, ascites, blood, breast milk, bone marrow, or urine) comprising obtaining a liquid biopsy sample from the subject; capturing TEX in the liquid biopsy sample using an antibody (such as, for example JAA-F11) specific to the TEX biomarker (such as, for example TF-Ag-α); detecting surface plasmon resonance (SPR) signal generated by the capture of TEX; measuring the expression of TEX biomarker based on SPR signal; analyzing the signal between the sample with TEX biomarker and reference sample, wherein the reference sample is obtained from a healthy subject; and administering a therapeutically effective dose of a humanized TEX biomarker specific antibody to the subject with a higher TEX biomarker expression compared to the reference sample from a healthy subject. For example, in one aspect, disclosed herein are methods for determining a TEX biomarker in a subject comprising: obtaining a sample from the subject via liquid biopsy (including, but not limited to liquid biopsy of cerebrospinal fluid, saliva, amniotic fluid, ascites, blood, breast milk, bone marrow, or urine); using an antibody to the TF-Ag-α to capture the TEX in the sample; using SPR to measure the levels of TF-Ag-α on captured TEX; using a second fluorescence labeled antibody that binds to second TEX biomarker (for example, a marker associated with a therapy, including, but not limited to PD-L1); using surface plasmon enhanced fluorescence (SPEF) spectrometry to measure the expression of second TEX marker; analyzing the signal between the sample with TF-Ag-α and the second TEX biomarker and the reference sample, wherein the reference sample is obtained from a healthy subject; determining the TEX biomarker expression; administering a therapeutically effective dose of a humanized TEX biomarker specific antibody to the subject with TEX biomarker expression which is different when compared to the reference sample from a healthy subject.

Also disclosed herein in one aspect are methods of detecting a TEX biomarker of any preceding aspect, wherein the biochip further enables multiplex detection of additional TEX biomarkers co-expressed on TEX, wherein the additional TEX biomarkers comprise PD-L1, PD-1, LAG-3, TIM-3, or CTLA-4.

In one aspect are methods of detecting a TEX biomarker of any preceding aspect, wherein the expression level of the TF-Ag-α in the sample is calculated using following equation:

$$Expression of TEX TF - Ag - α = {( \frac{{I}_{TEX TF - Ag - α} - {I}_{PBS}}{{I}_{PBS} - {I}_{water}} )}_{JAA - F 11 antibody modified biochip} - {( \frac{{I}_{TEX TF - Ag - α} - {I}_{PBS}}{{I}_{PBS} - {I}_{water}} )}_{IgG 3 control antibody modified biochip}$$

wherein water and PBS emit baseline signal; wherein IgG3 is a control antibody; wherein a higher expression level of TEX TF-Ag-α in the sample relative to the reference sample indicates presence of a cancer.

Also described herein are systems to detect expression level of a TEX biomarker (including, but not limited to exosomes derived from lung cancer, breast cancer, prostate cancer, colon cancer, ovarian cancer, or bladder cancer) in a subject using liquid biopsy (including, but not limited to cerebrospinal fluid, saliva, amniotic fluid, ascites, blood, breast milk, bone marrow, or urine), comprising: a biochip for detection of TEX biomarker (such as, for example, a protein, a nucleic acid, a carbohydrate or a lipid including, but not limited to TF-Ag-α) in a liquid biopsy sample; an antibody (such as, for example JAA-FT 1) immobilized on the biochip, wherein the antibody is specific to the TEX biomarker; a biosensor (photodetector) to record a SPR signal from the liquid biopsy sample; wherein the SPR signal is obtained when TEX biomarker binds to the antibody; and an equation to analyze expression level of the TEX biomarker, wherein a high TEX biomarker expression compared to a reference sample indicates presence of a cancer.

Also disclosed herein are systems of any preceding aspect wherein the expression level of TEX TF-Ag-α in the sample is calculated using the equation:

$$Expression of TEX TF - Ag - α = {( \frac{{I}_{TEX TF - Ag - α} - {I}_{PBS}}{{I}_{PBS} - {I}_{water}} )}_{JAA - F 11 antibody modified biochip} - {( \frac{{I}_{TEX TF - Ag - α} - {I}_{PBS}}{{I}_{PBS} - {I}_{water}} )}_{IgG 3 control antibody modified biochip}$$

wherein water and PBS emit baseline signal; wherein IgG3 is a control antibody; wherein a higher expression level of TEX TF-Ag-α in the sample relative to the reference sample indicates presence of a cancer and the successful capture of TEX.

In one aspect, disclosed herein are methods to assess the efficacy of a cancer immunotherapy response in a liquid biopsy comprising: obtaining a liquid biopsy (including, but not limited to cerebrospinal fluid, saliva, amniotic fluid, ascites, blood, breast milk, bone marrow, or urine); capturing TEX using TEX biomarker specific antibody (first antibody) (such as, for example JAA-F11) attached to a biochip; detecting a TEX biomarker (such as, for example, TF-Ag-α) in the liquid biopsy by measuring the SPR related signal using an SPR biosensor; measuring TEX biomarker expression; exposing the biochip to a second antibody specific to a biomarker associated with response to an immune checkpoint inhibitor (such as, for example, anti-PD-1, anti-PD-L1, anti-LAG3, anti-TIM3 or anti-CTLA-4) co-expressed on TEX; measuring an SPEF signal related to the biomarker associated with response to an immune checkpoint inhibitor (ICI); measuring an SPEF signal related to TEX biomarker associated with the response to ICI (TEX-ICI); and administering a therapeutically effective dose of an ICI to a subject with increased expression of the TEX biomarker relative to a subject with no or decreased expression of TEX. For example, disclosed herein, in one aspect is a method to determine cancer immunotherapy response comprising: obtaining a sample from a subject via liquid biopsy (including, but not limited to liquid biopsy of cerebrospinal fluid, saliva, amniotic fluid, ascites, blood, breast milk, bone marrow, or urine); using an antibody to TF-Ag-α attached to a biochip to capture TEX expressing TF-Ag-α (i.e., TEX^{TF-Ag-α}); using SPR to measure the levels of TF-Ag-α on captured TEX^{TF-Ag-α}; using fluorescence labeled antibodies to bind to biomarkers associated with response to immunotherapy on the captured TEX^{TF-Ag-α}; using surface plasmon enhanced (SPEF) spectrometry to measure the levels of biomarker associated with immunotherapy response on TEX^{TF-Ag-α} (TEX^{TF-Ag-α}-ICI), wherein ICI is a type of cancer immunotherapy; analyzing the TEX^{TF-Ag-α}-ICI expression between the sample and a reference sample, wherein the reference sample is obtained from the same subject prior to treatment (pre-treatment baseline) or at the end of prior treatment cycle; calculating expression of TEX^{TF-Ag-α}-ICI; and administering a therapeutically effective dose of the ICI to the subject with increased expression of TEX^{TF-Ag-α}-ICI in contrast to the subject with no change or decreased expression of TEX^{TF-Ag-α}-ICI. In one aspect, the ICI comprises anti-immune checkpoint proteins monoclonal antibodies, wherein the monoclonal antibodies are anti-PD-1, anti-PD-L1, anti-LAG3, anti-TIM3 or anti CTLA-4.

Also disclosed herein are systems and methods to assess the efficacy of a cancer immunotherapy response of any preceding aspect, wherein the biomarker associated with immune checkpoint proteins co-expressed on TEX in the sample is calculated using following equation:

$$Expression of {TEX}^{TF - Ag - α} PD - L 1 = ( \frac{{I}_{after APC - PD - L 1} - {I}_{before APC - PD - L 1}}{{I}_{before APC - PD - L 1}} ) / ( Expression of TEX TF - Ag - α ) = ( \frac{{I}_{after APC - PD - L 1} - {I}_{before APC - PD - L 1}}{{I}_{before APC - PD - L 1}} ) / ( \frac{{I}_{TEX TF - Ag - α} - {I}_{PBS}}{{I}_{PBS} - {I}_{water}} )$$

wherein water and PBS emit baseline signal; wherein TF-Ag-α is a TEX biomarker; wherein TEX TF-Ag-α is the level of TF-Ag-α expressed on TEX; wherein TEX^{TF-Ag-α} is tumor-derived exosomes that express TF-Ag-α; wherein TEX^{TF-Ag-α} PD-L1 is the biomarker associated with immune checkpoint proteins co-expressed on TEX^{TF-Ag-α}; wherein PD-L1 is the biomarker associated with response to an ICI (for example, pembrolizumab); wherein APC is a fluorescent dye attached to anti-PD-L1 antibody; wherein after APC-PD-L1 is fluorescence intensity of APC after incubating biochip with anti-APC-PD-L1 antibody; and wherein before APC-PD-L1 is fluorescence intensity of APC before incubating biochip with anti-APC-PD-L1 antibody.

### BRIEF DESCRIPTION OF THE FIGS.

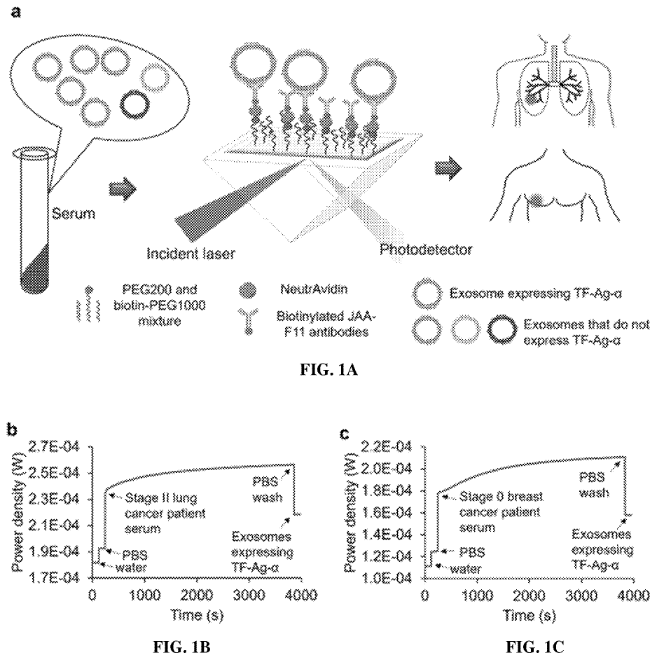

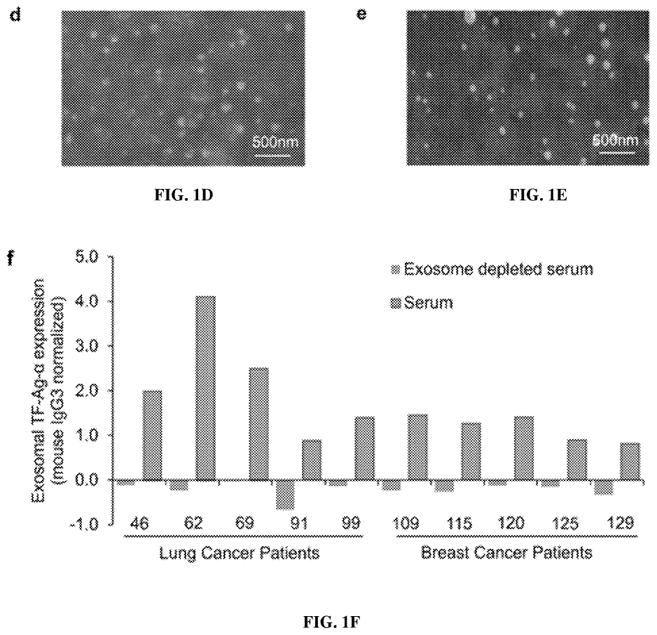

FIGS. 1A-1F show SPR assay detects exosomal TF-Ag-α for cancer diagnosis. FIG. 1A shows a sensing mechanism of SPR assay. Representative SPR curves for the detection of exosomal TF-Ag-α in serum samples from a lung cancer patient FIG. 1B and a breast cancer patient FIG. 1C. SEM images of exosomes expressing TF-Ag-α captured on the biochips from serum samples of a lung cancer patient FIG. 1D and a breast cancer patient FIG. 1E. FIG. 1F shows a little signal was detected in exosome-depleted serum samples, but strong signals were observed in the same serum samples without exosome depletion (n=10, patient characteristics was provided in TABLE 5 and TABLE 6).

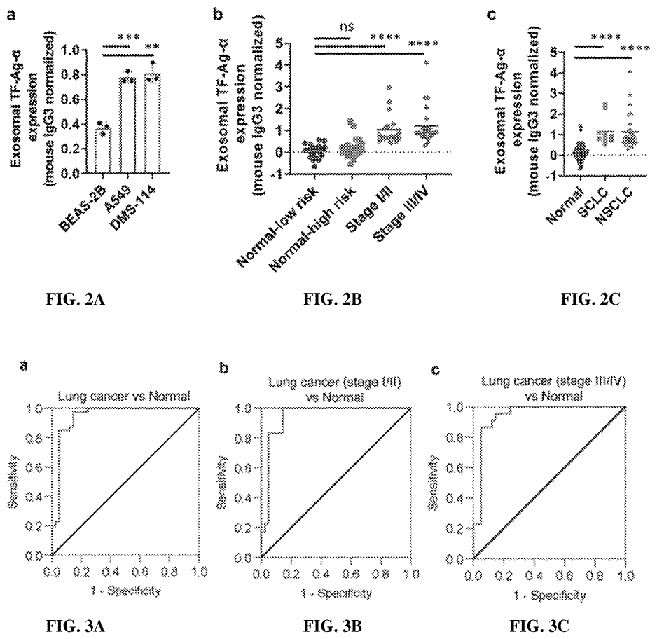

FIGS. 2A-2C show evaluation of exosomal TF-Ag-α in lung cancer diagnosis. FIG. 2A shows expression of TF-Ag-α was significantly higher in exosomes from A549 NSCLC cells and DMS-114 small cell lung cancer (SCLC) cells than those from BEAS-2B normal bronchial epithelial cells at exosome concentration of 4×10¹⁰ exosomes/mL. FIG. 2B shows the expression of exosomal TF-Ag-α in serum samples from a training set of lung cancer patients and normal controls was measured. Exosomal TF-Ag-α expression was significantly higher in serum samples from lung cancer patients at all stages (n=40; stage I/II: n=18; stage III/IV: n=22) than normal controls (n=41; low-risk: n=20; high-risk: n=21). FIG. 2C shows exosomal TF-Ag-α expression was significantly higher in serum samples from NSCLC (n=30) and SCLC (n=10) than normal controls (n=41). **p<0.01, ***p<0.001, ****p<0.0001.

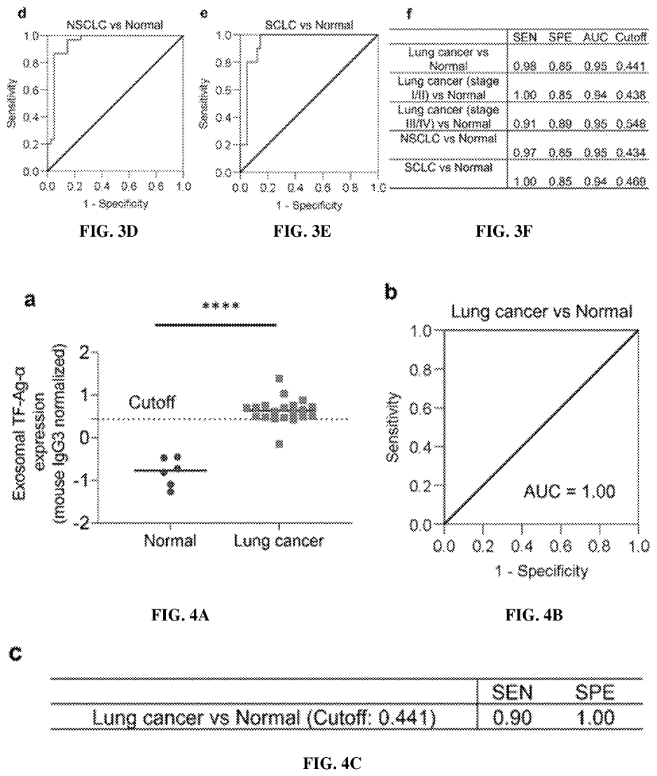

FIGS. 3A-3F show receiver operating characteristics (ROC) analysis to evaluate diagnostic performance of exosomal TF-Ag-α in lung cancer. ROC curves of comparisons between all lung cancer patients (n=40) and normal controls (n=41) FIG. 3A, early stage lung cancer (stage I/II, n=18) and normal controls (n=41) FIG. 3B, late stage lung cancer (stage III/IV, n=22) and normal controls (n=41) FIG. 3C, NSCLC (n=30) and normal controls (n=41) FIG. 3D, and SCLC (n=10) and normal controls (n=41) FIG. 3E shows normal controls included 20 low-risk patients and 21 high-risk patients. The sensitivity (SEN), specificity (SPE), area under curve (AUC) and cutoff values of exosomal TF-Ag-α for each comparison were summarized in FIG. 3F.

FIGS. 4A-4C show a blind evaluation of exosomal TF-Ag-α in lung cancer diagnosis using a test set of lung cancer patients and controls. FIG. 4A shows exosomal TF-Ag-α expression was significantly higher in serum samples from lung cancer patients at all stages (total n=20; stage I/II: n=10; stage III/IV: n=10) than normal controls (n=6). (****: p<0.0001) FIG. 4B shows ROC curve of comparison between lung cancer patients (n=20) and normal controls (n=6). Area under curve (AUC) was 1.00. FIG. 4C shows sensitivity (SEN) and specificity (SPE) of exosomal TF-Ag-α in distinguishing lung cancer patients from normal controls using cutoff value of 0.441 obtained from the training set.

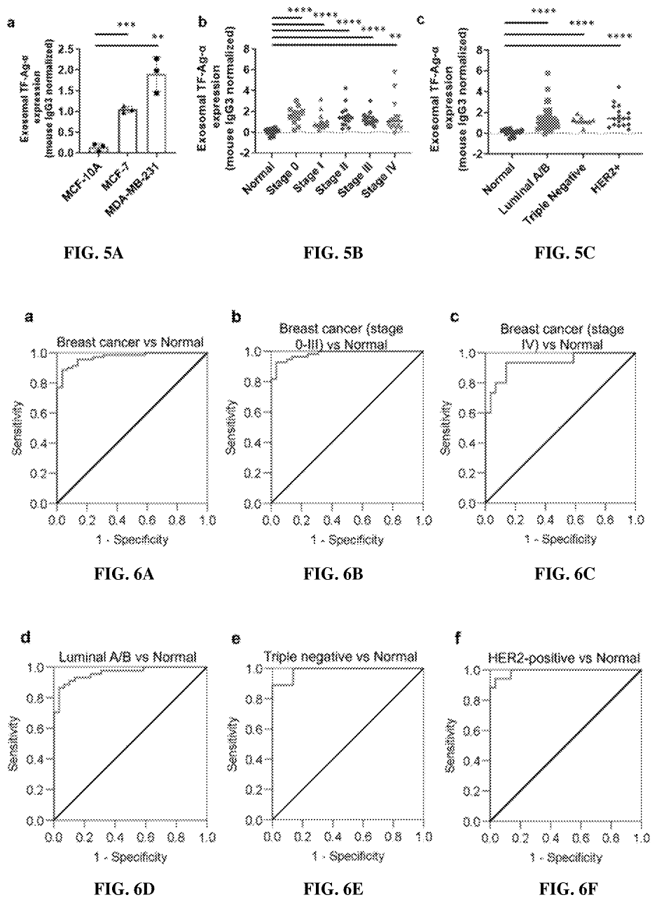

FIGS. 5A-5C show evaluation of exosomal TF-Ag-α in breast cancer diagnosis. FIG. 5A shows expression of TF-Ag-α was significantly higher in exosomes from MDA-MB-231 triple-negative breast cancer cells and MCF-7 luminal A breast cancer cells than those from MCF-10A normal breast epithelia cells at exosome concentration of 10¹¹ exosomes/mL. FIG. 5B shows the levels of exosomal TF-Ag-α in serum samples from a training set of breast cancer patients and normal controls were measured. Exosomal TF-Ag-α expression was significantly higher in serum samples from breast cancer patients at all stages (n=70; stage 0: n=12; stage I: n=14; stage II: n=15, stage III: n=14; stage IV: n=15) than normal controls (n=14) and patients with benign breast conditions (n=15). FIG. 5C shows exosomal TF-Ag-α expression was significantly higher in serum samples from luminal A/B (n=44), triple negative (n=9) and HER2-positive (n=17) breast cancer than normal controls (n=29 including 15 women with benign breast conditions). **p<0.01, ***p<0.001, ****p<0.0001.

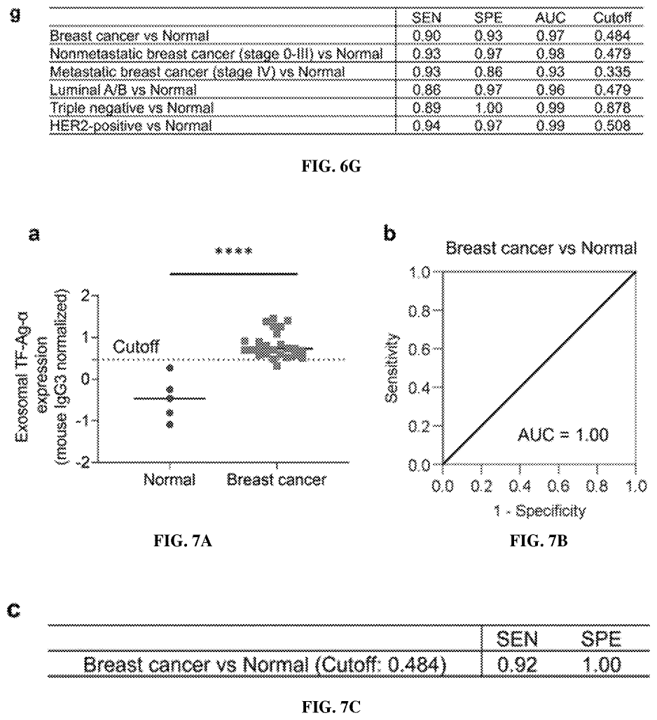

FIGS. 6A-6G show receiver operating characteristics (ROC) analysis to evaluate diagnostic performance of exosomal TF-Ag-α in breast cancer. ROC curves of comparisons between all breast cancer patients (n=70) and normal controls (n=29) FIG. 6A, nonmetastatic breast cancer (stage 0-III, n=55) and normal controls (n=29) FIG. 6B, metastatic breast cancer (stage IV, n=15) and normal controls (n=29) FIG. 6C, luminal A/B breast cancer (n=44) and normal controls (n=29) FIG. 6D, triple negative breast cancer (n=9) and normal controls (n=29) FIG. 6E, and HER2-positive breast cancer (n=17) and normal controls (n=29) FIG. 6F. The sensitivity (SEN), specificity (SPE) and area under curve (AUC), and cutoff values of exosomal TF-Ag-α for each comparison were summarized in FIG. 6G.

FIGS. 7A-7C show a blind evaluation of exosomal TF-Ag-α in breast cancer diagnosis using a test set of breast cancer patients and controls. FIG. 7A shows exosomal TF-Ag-α expression was significantly higher in serum samples from breast cancer patients at all stages (total n=25; stage 0-IV, 5 per stage) than normal controls (n=5 including 2 patients with benign breast conditions). (****: p<0.0001) FIG. 7B shows ROC curve of comparison between breast cancer patients (n=25) and normal controls (n=5). Area under curve (AUC) was 1.00. FIG. 7C shows sensitivity (SEN) and specificity (SPE) of exosomal TF-Ag-α in distinguishing breast cancer patients from normal controls using cutoff value of 0.484 obtained from the training set.

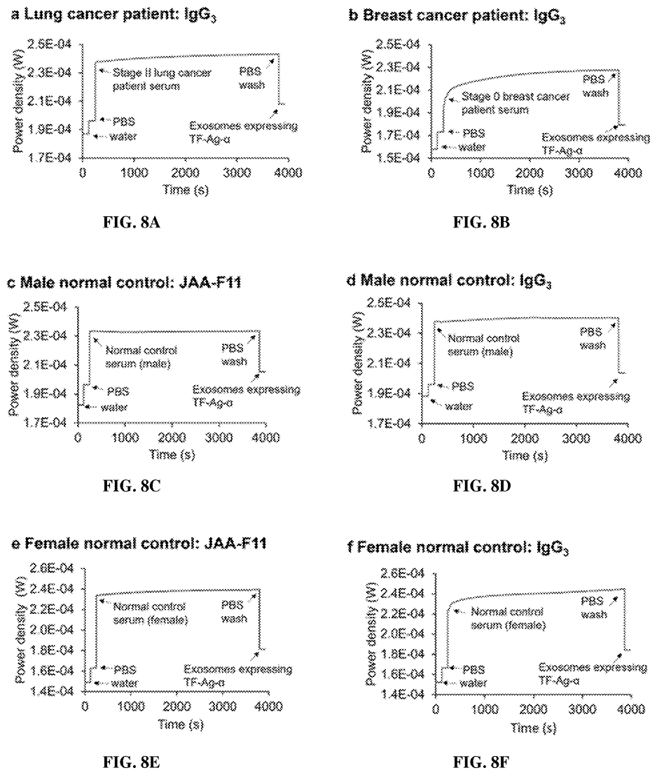

FIGS. 8A-8F show SPR assay detects exosomal TF-Ag-α for cancer diagnosis. Representative SPR curves for the detection of exosomal TF-Ag-α in serum samples from cancer patients and controls. FIG. 8A shows a Stage II lung cancer patient using a biochip modified with IgG3 negative control antibodies; FIG. 8B shows a Stage 0 breast cancer patient using a biochip modified with IgG3 negative control antibodies, FIG. 8C shows a male normal control using a biochip modified with JAA-F11 antibodies, FIG. 8D shows a male normal control using a biochip modified with IgG3 negative control antibodies, FIG. 8E shows a female normal control using a biochip modified with JAA-F11 antibodies, and FIG. 8F a female normal control using a biochip modified with IgG3 negative control antibodies.

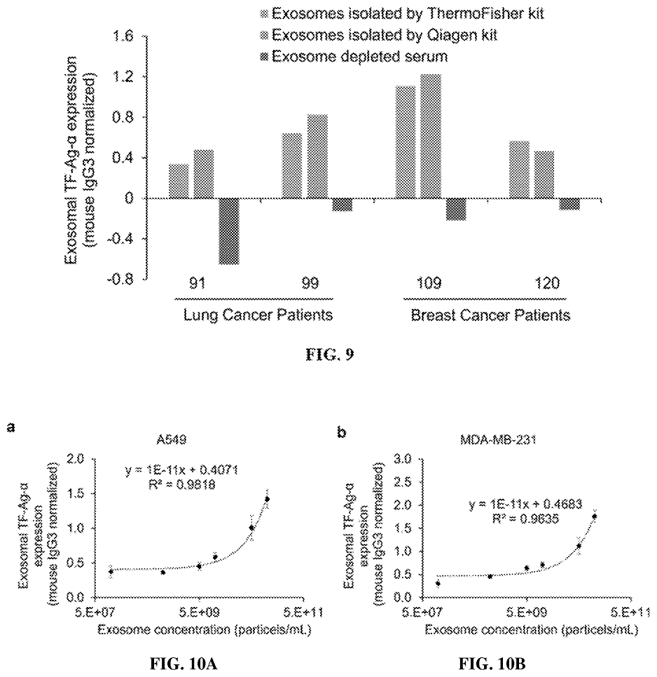

FIG. 9 shows SPR assay demonstrated exosomes carry TF-Ag-α. Exosomes were isolated from cancer patient serum samples using both total exosome isolation kit (from serum) (THERMO FISHER SCIENTIFIC®) and exoRNeasy midi kit (Qiagen®). Significantly higher levels of TF-Ag-α were observed in exosome samples isolated using both kits than the exosome-depleted serum samples. The exosome concentration was 3.29×10¹¹ exosomes/mL.

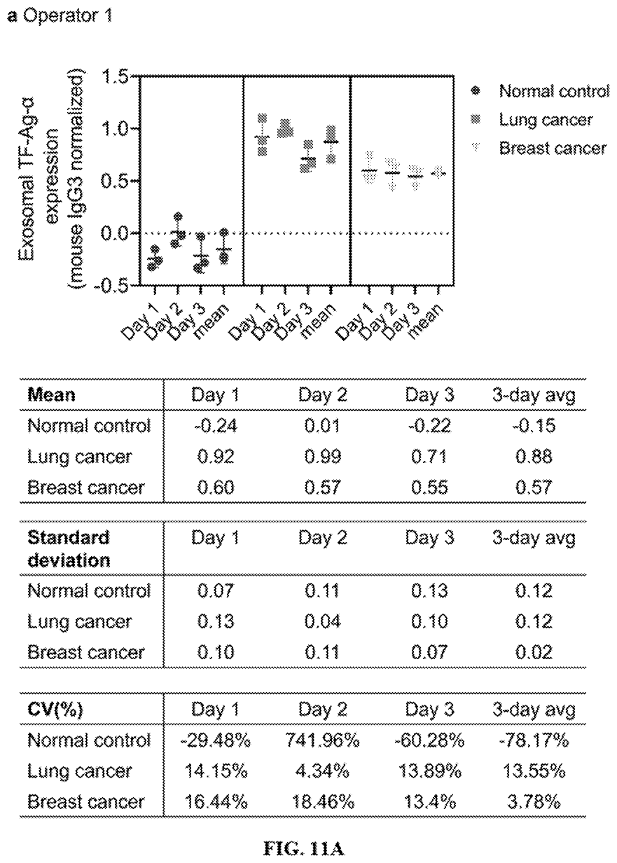

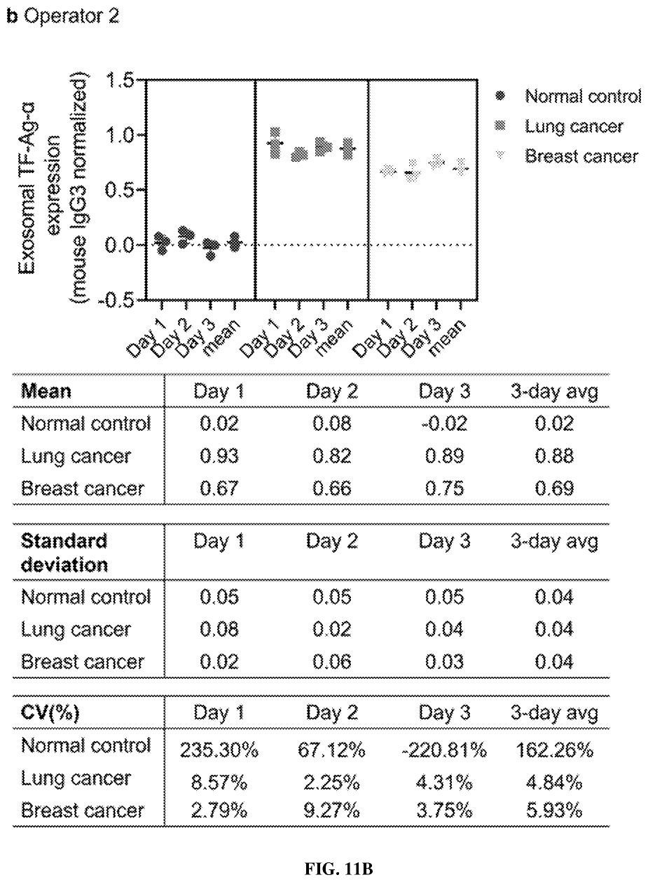

FIGS. 10A-10B show sensing performance of SPR assay in detecting exosomal TF-Ag-α. Exosomes from A549 NSCLC cells FIG. 10A and MDA-MB-231 breast cancer cells FIG. 10B were spiked in the serum of a normal control at concentrations of 0 to 10¹¹ exosomes/mL. The expression of exosomal TF-Ag-α was measured using the SPR assay. For A549 cell-derived exosomes, the LOD was 5×10⁹ exosomes/mL and the linear range was from 5×10⁹ to 10¹¹ exosomes/mL. For MDA-MB-231 cell-derived exosomes, the LOD was 10⁹ exosomes/mL and the linear range was from 10⁹ to 10¹¹ exosomes/mL.

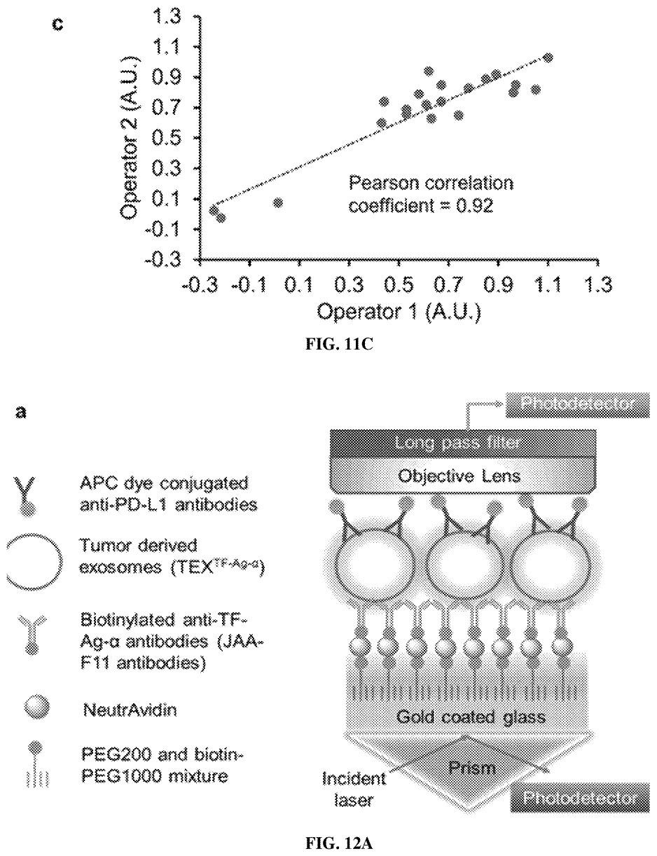

FIGS. 11A-11C show repeatability and reproducibility of SPR assay in detecting exosomal TF-Ag-α. The levels of exosomal TF-Ag-α in serum samples from one normal control, one lung cancer patient and one breast cancer patient were measured by operator 1 FIG. 11A and operator 2 FIG. 11B on 3 different days, 3 replicates per day. The mean, standard deviation, and 16 coefficient of variation (CV) were reported. FIG. 11C shows a Pearson correlation of results from operator 1 and operator 2.

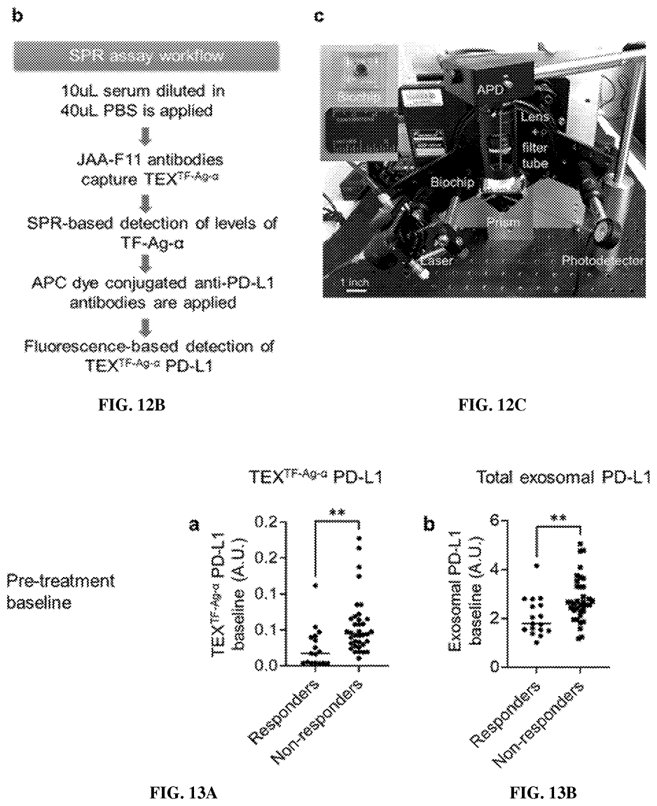

FIGS. 12A-12C show SPR based liquid biopsy assay that selectively captures TEX^{TF-Ag-α} and measures TEX^{TF-Ag-α} PD-L1. The schematic diagram FIG. 12A, the workflow FIG. 12B and a photo FIG. 12C of the SPR based liquid biopsy assay. This shows the prognostic ability of the present disclosure.

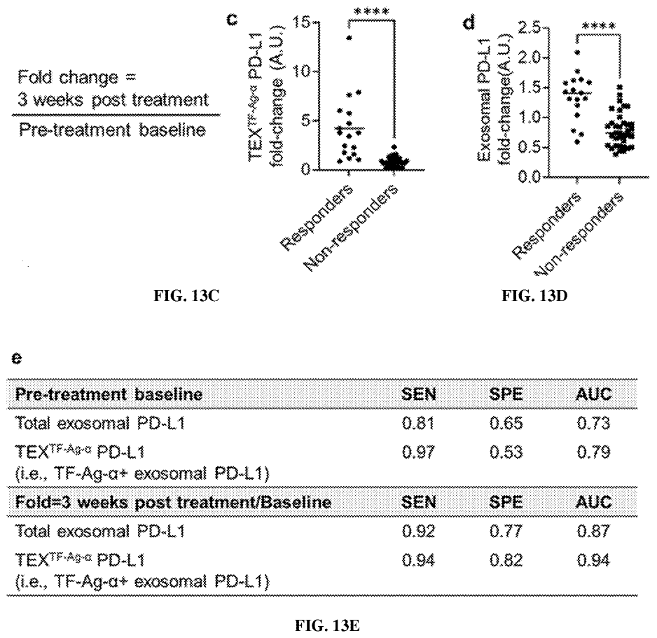

FIGS. 13A-13E show TEX^{TF-Ag-α} PD-L1 better distinguished responders (n=17) from non-responders (n=36) than total exosomal PD-L1. Levels of TEX^{TF-Ag-α} PD-L1 (FIG. 13A) and total exosomal PD-L1 (FIG. 13B) in serum samples collected at pre-treatment baseline. Fold change of TEX^{TF-Ag-α} PD-L1 (FIG. 13C) and total exosomal PD-L1 (FIG. 13D) in serum samples collected at 3 weeks post treatment. FIG. 13E shows ROC analysis showed that TEX^{TF-Ag-α} PD-L1 showed higher sensitivity (SEN), specificity (SPE) and area under curve (AUC) than total exosomal PD-L1 in distinguishing responders from non-responders at both pre-treatment baseline and 3 weeks post treatment. (**: p<0.01; ****: p<0.0001). This shows the prognostic ability of the present disclosure.

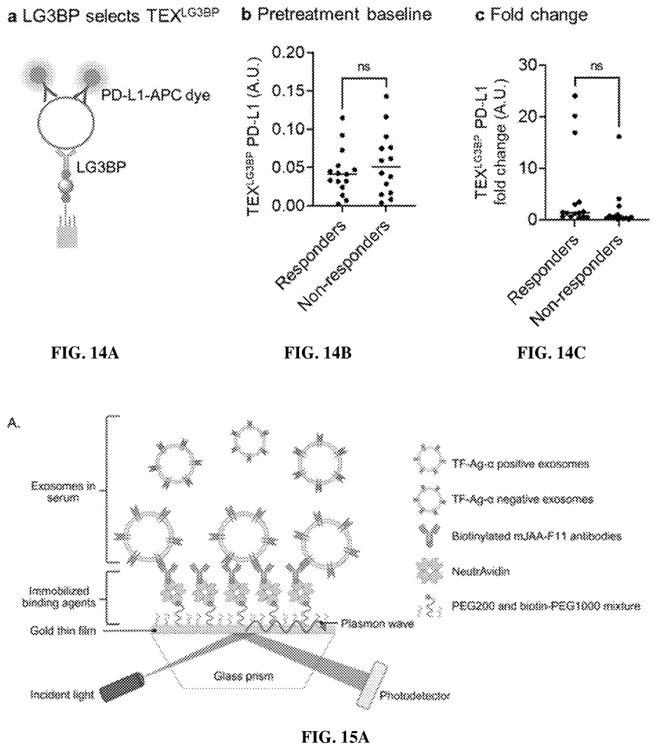

FIGS. 14A-14C show LG3BP is not an effective marker to select TEX. FIG. 14A shows TEX^{LG3BP} were selectively captured by anti-LG3BP antibody and the TEX^{LG3BP} PD-L1 levels were measured. FIG. 14B shows expression of TEX^{LG3BP} PD-L1 in serum samples collected at pre-treatment baseline. FIG. 14C shows fold change of TEX^{LG3BP} PD-L1 in serum samples collected at 3 weeks post treatment. No significant difference in the expression of baseline TEX^{LG3BP} PD-L1 and fold change of TEX^{LG3BP} PD-L1 was observed between responders (n=15) and non-responders (n=14), suggesting that LG3BP is inferior to TF-Ag-α in selectively capture TEX. This shows the prognostic ability of the present disclosure.

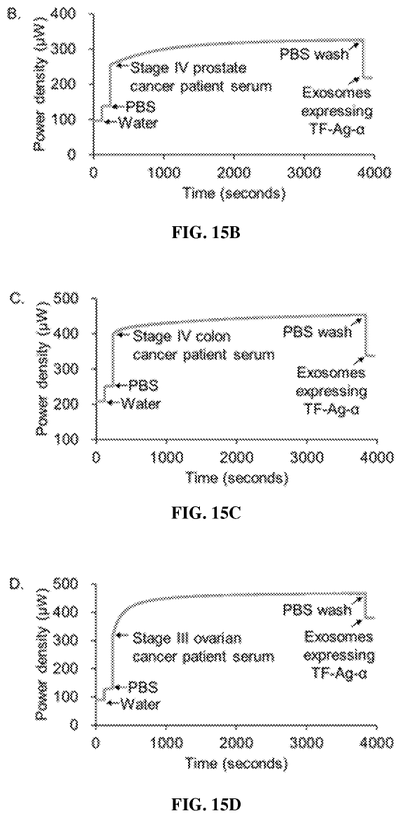

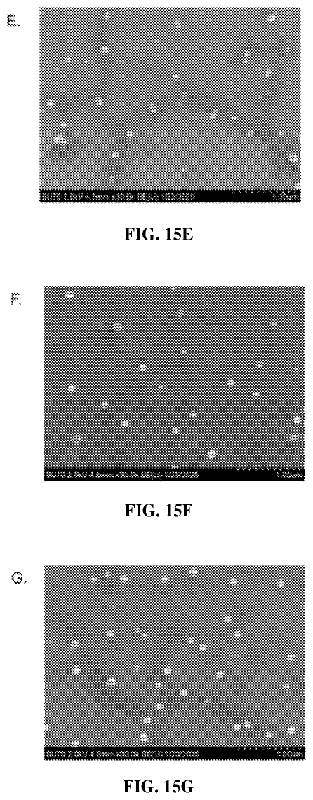

FIGS. 15A-15G show SPR assay detects exosomal TF-Ag-α for prostate, colon and ovarian cancer diagnosis. FIG. 15A shows a schematic illustration of SPR-based detection of exosomal TF-Ag-α. FIGS. 15B-15D show representative SPR curves for the detection of exosomal TF-Ag-α in serum samples from patients with prostate cancer (FIG. 15B), colon cancer (FIG. 15C), and ovarian cancer (FIG. 15D). FIGS. 15E-15G show SEM images of TF-Ag-α-positive exosomes captured on the biochips from serum samples of patients with prostate cancer (FIG. 15E), colon cancer (FIG. 15F), and ovarian cancer (FIG. 15G).

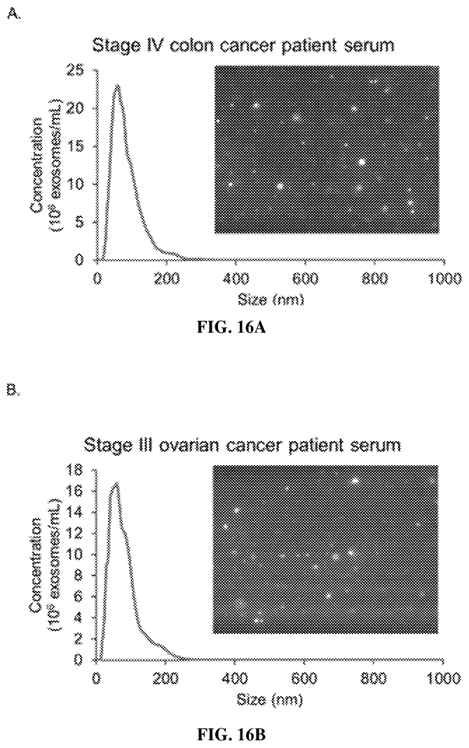

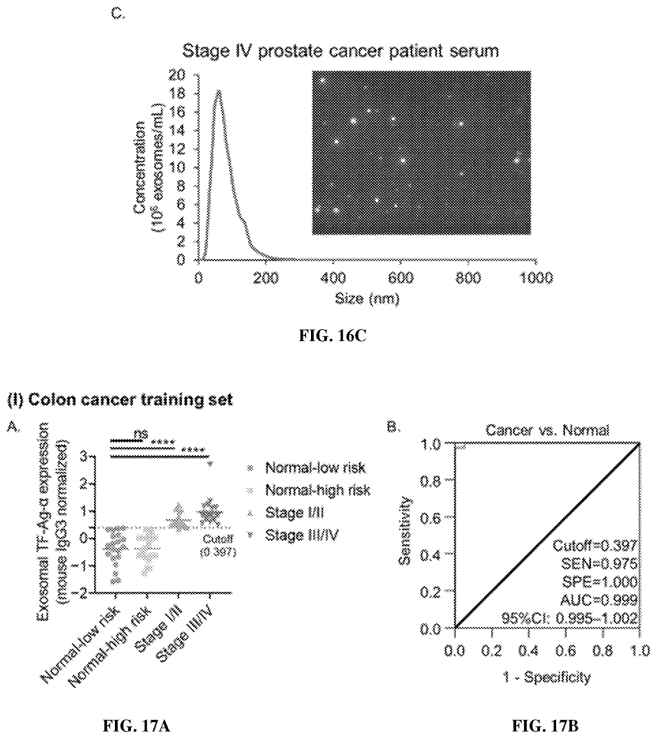

FIGS. 16A-16C show characterization of exosomes isolated from patient serum samples via nanoparticle tracking analysis (NTA). FIG. 16A shows a representative size distribution curve of exosomes isolated from 100 uL serum of a stage IV colon cancer patient (10,000× dilution). The mean diameter of exosomes was 85.56 nm (n=3). FIG. 16B shows a representative size distribution of exosomes isolated from 100 uL serum of a stage III ovarian cancer patient (1,000× dilution). The mean diameter of exosomes was 80.52 nm (n=3). FIG. 16C shows a representative size distribution of exosomes isolated from 100 uL serum of a stage IV prostate cancer patient (10,000× dilution). The mean diameter of exosomes was 81.16 nm (n=3). Insert: Screenshot from the recorded video of exosomes during NTA.

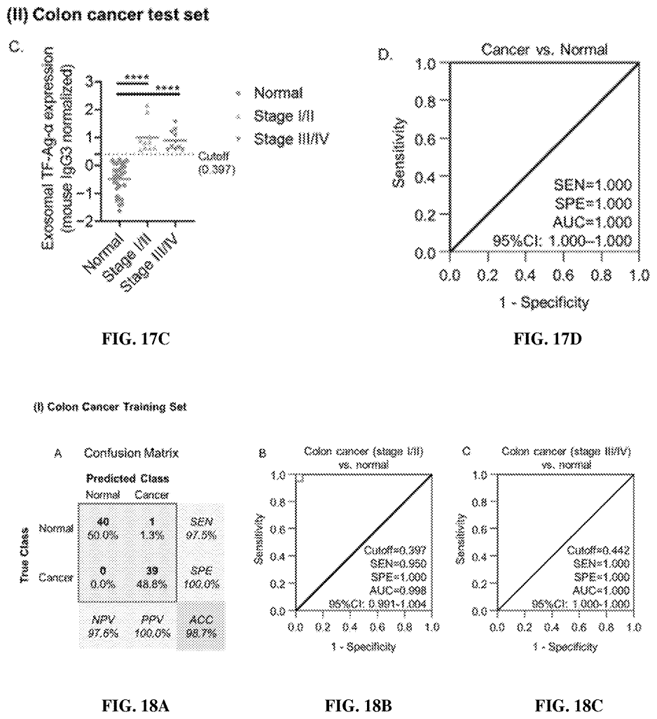

FIGS. 17A-17D show evaluation of exosomal TF-Ag-α for colon cancer diagnosis using a training set and an independent test set. FIG. 17A is a scatterplot showing serum exosomal TF-Ag-α levels in colon cancer patients (n=40; stage I/II: n=20; stage III/IV: n=20) and normal controls (n=40; low risk: n=20; benign conditions at high risk of cancer: n=20) in the training set. FIG. 17B shows ROC curve analysis comparing colon cancer patients vs. normal controls in the training set. FIG. 17C is a scatterplot showing serum exosomal TF-Ag-α levels in colon cancer patients (n=20; stage I/II: n=10; stage III/IV: n=10) and normal controls (n=29) in the test set. FIG. 17D shows ROC curve analysis comparing colon cancer patients vs. normal controls in the test set. (SEN: sensitivity, SPE: specificity, AUC: area under the curve. ns: not significant; ****: p<0.0001).

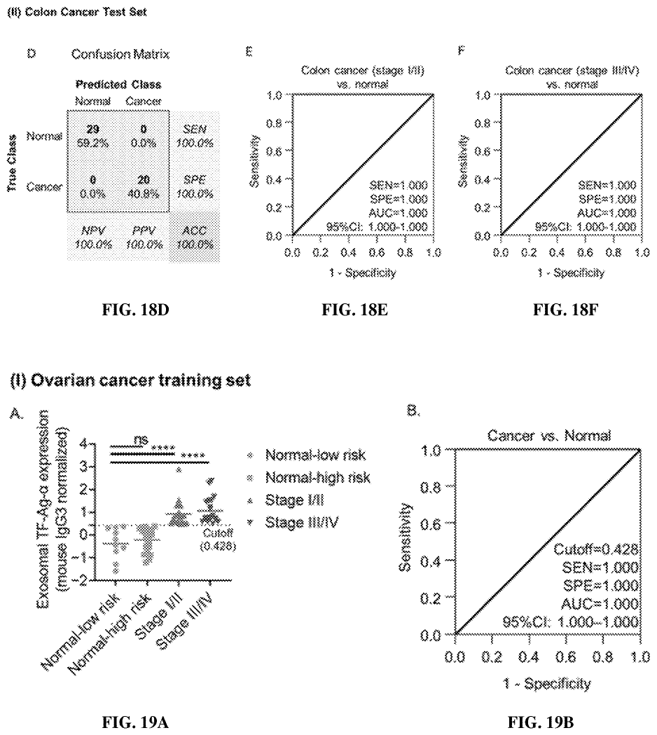

FIGS. 18A-18F show evaluation of exosomal TF-Ag-α for colon cancer diagnosis using a training set and an independent test set. FIG. 18A is a confusion matrix showing the diagnostic performance of exosomal TF-Ag-α in colon cancer based on the training set which included 40 colon cancer patients (stage I/II: n=20; stage III/IV: n=20) and 40 normal controls (low risk: n=20; benign conditions at high risk of cancer: n=20). FIG. 18B shows ROC curve analysis comparing early-stage colon cancer patients (Stage I/II, n=20) vs. normal controls (n=40) in the training set. FIG. 18C shows ROC curve analysis comparing late-stage colon cancer patients (Stage III/IV, n=20) vs. normal controls (n=40) in the training set. FIG. 18D is a confusion matrix showing the diagnostic performance of exosomal TF-Ag-α based on the test set which included 20 colon cancer patients (stage II: n=10; stage III/IV: n=10) and 29 normal controls. FIG. 18E shows ROC curve analysis comparing early-stage colon cancer patients (Stage I/II, n=10) vs. normal controls (n=29) in the test set. FIG. 18F shows ROC curve analysis comparing late-stage colon cancer patients (Stage III/IV, n=10) vs. normal controls (n=29) in the test set. (SEN: sensitivity, SPE: specificity, NPV: negative predictive value; PPV: positive predictive value, ACC: overall accuracy, AUC: area under the curve.)

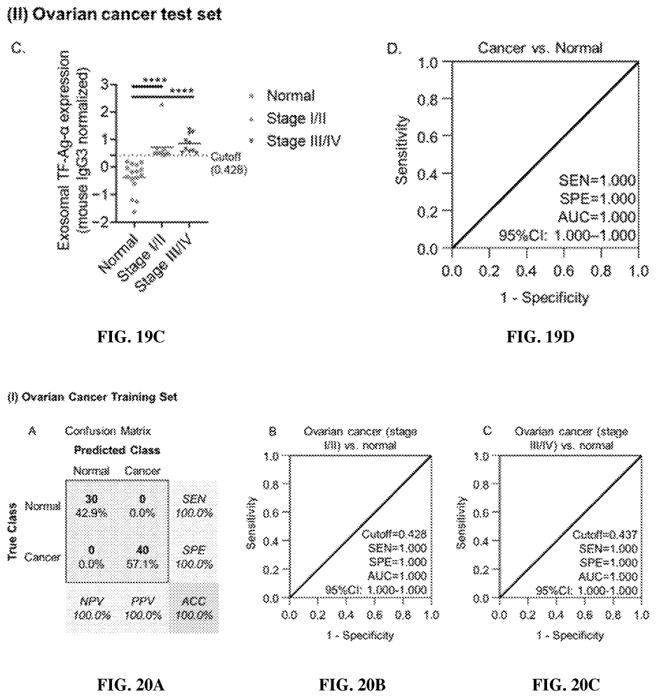

FIGS. 19A-19D show evaluation of exosomal TF-Ag-α in ovarian cancer diagnosis using a training set and an independent test set. FIG. 19A shows a scatterplot of serum exosomal TF-Ag-α levels in ovarian cancer patients (n=40; n=10 for each stage) and normal controls (n=30; low risk: n=10; high risk: n=20) in the training set. FIG. 19B shows ROC curve analyses comparing ovarian cancer patients vs. normal controls in the training set. FIG. 19C shows a scatterplot of exosomal TF-Ag-α levels in serum samples from ovarian cancer patients (n=20; stage I/II: n=10; stage III/IV: n=10) and normal controls (n=16) in the test set. FIG. 19D shows ROC curves comparing ovarian cancer patients and normal controls in the test set. (SEN: sensitivity, SPE: specificity, AUC: area under the curve. ns: not significant; ****: p<0.0001.).

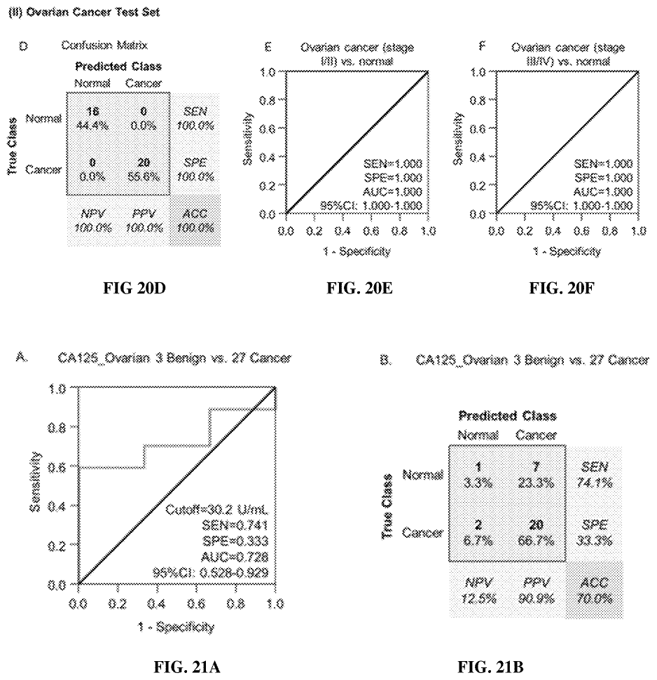

FIGS. 20A-20F show evaluation of exosomal TF-Ag-α for ovarian cancer diagnosis using a training set and an independent test set. FIG. 20A is a confusion matrix showing the diagnostic performance of exosomal TF-Ag-α in ovarian cancer based on the training set which included 40 ovarian cancer patients (stage I/II: n=20; stage III/IV: n=20) and 30 normal controls (low risk: n=10; benign conditions at high risk of cancer: n=20). FIG. 20B shows ROC curve analysis comparing early-stage ovarian cancer patients (Stage I/II, n=20) vs. normal controls (n=30) in the training set. FIG. 20C shows ROC curve analysis comparing late-stage ovarian cancer patients (Stage III/IV, n=20) vs. normal controls (n=30) in the training set. FIG. 20D is a confusion matrix showing the diagnostic performance of exosomal TF-Ag-α based on the test set which included 20 ovarian cancer patients (stage I/II: n=10; stage III/IV: n=10) and 16 normal controls. FIG. 20E shows ROC curve analysis comparing early-stage ovarian cancer patients (Stage I/II, n=10) vs. normal controls (n=16) in the test set. FIG. 20F shows ROC curve analysis comparing late-stage ovarian cancer patients (Stage III/IV, n=10) vs. normal controls (n=16) in the test set. (SEN: sensitivity, SPE: specificity, NPV: negative predictive value; PPV: positive predictive value, ACC: overall accuracy, AUC: area under the curve.)

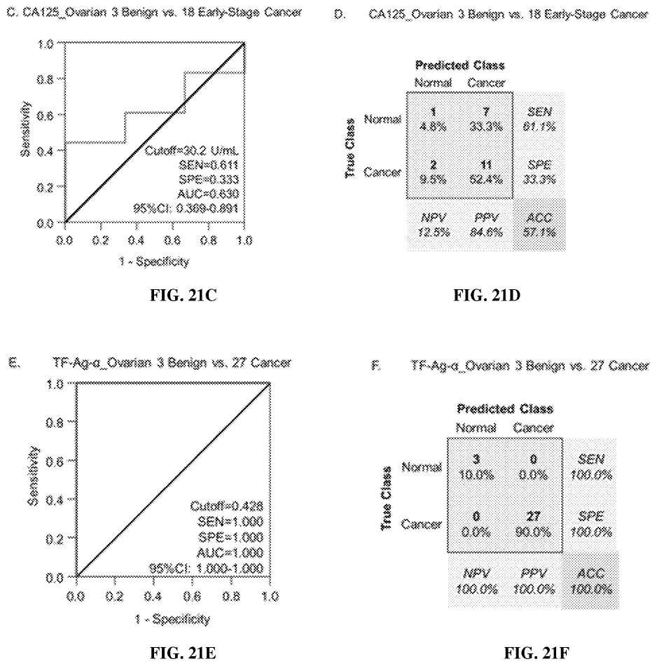

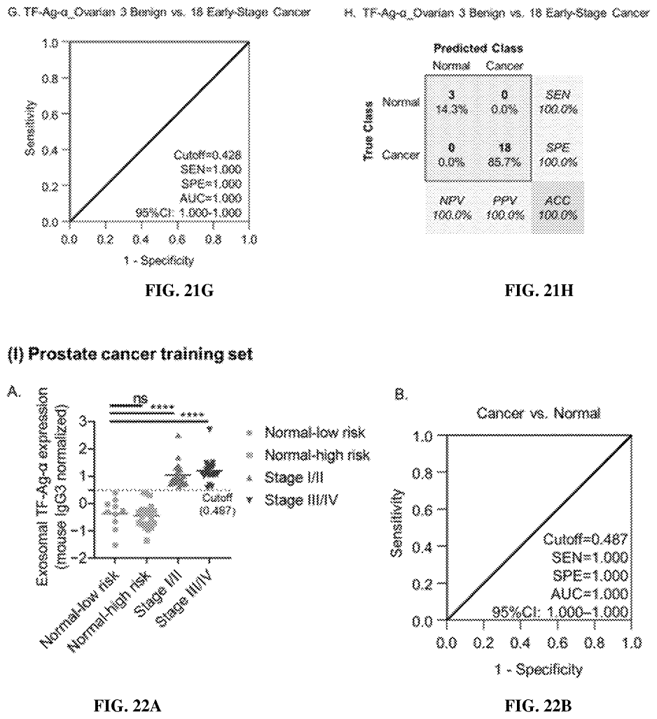

FIGS. 21A-21H show diagnostic performance comparison between CA125 and exosomal TF-Ag-α in ovarian cancer. Pre-treatment CA125 data were available for a subset of ovarian cancer patients (n=27) and patients with benign conditions (n=3), with blood samples collected within 100 days of those used for exosomal TF-Ag-α analysis to maintain temporal and analytical consistency. FIG. 21A shows ROC curve analysis comparing patients with ovarian cancer (n=27, stage I-IV) vs. benign conditions (n=3) using CA125 as the biomarker. FIG. 21B is a confusion matrix illustrating the diagnostic performance of CA125 based on 27 ovarian cancer patients and 3 patients with benign conditions. FIG. 21C shows ROC curve analysis comparing patients with early-stage ovarian cancer (n=18, stage I-II) vs. benign conditions (n=3) using CA125 as the biomarker. FIG. 21D is a confusion matrix showing diagnostic performance of CA125 based on 18 early-stage ovarian cancer patients and 3 patients with benign conditions. FIG. 21E shows ROC curve analysis comparing patients with ovarian cancer (n=27, stage I-IV) vs. benign conditions (n=3) using exosomal TF-Ag-α as the biomarker. FIG. 21F is a confusion matrix showed diagnostic performance of exosomal TF-Ag-α based on 27 ovarian cancer patients and 3 patients with benign conditions. FIG. 21G shows ROC curve analysis comparing patients with early-stage ovarian cancer (n=18, stage I-II) vs. benign conditions (n=3) using exosomal TF-Ag-α as the biomarker. FIG. 21H is a confusion matrix showed diagnostic performance of exosomal TF-Ag-α based on 18 early-stage ovarian cancer patients and 3 patients with benign conditions.

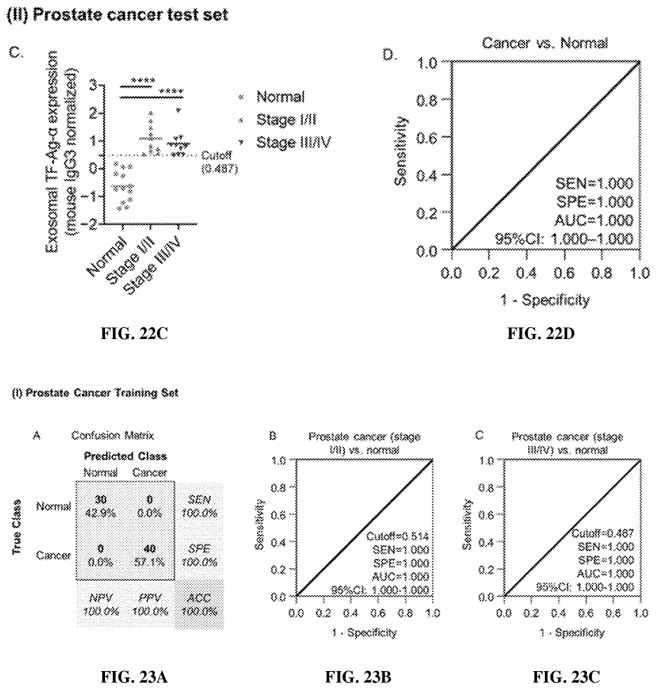

FIGS. 22A-22D show evaluation of exosomal TF-Ag-α for prostate cancer diagnosis using a training set and an independent test set. FIG. 22A is a scatterplot showing serum exosomal TF-Ag-α levels in prostate cancer patients (n=40; stage II: n=20; stage III/IV: n=20) and normal controls (n=30; low risk: n=10; high risk: n=20) in the training set. FIG. 22B shows ROC curve analysis comparing prostate cancer patients vs. normal controls in the training set. FIG. 22C is a scatterplot showing serum exosomal TF-Ag-α levels in prostate cancer patients (n=20; stage I/II: n=10; stage III/IV: n=10) and normal controls (n=13) in the test set. FIG. 22D shows ROC curve analysis comparing prostate cancer patients vs. normal controls in the test set. (SEN: sensitivity, SPE: specificity, AUC: area under the curve. ns: not significant; ****: p<0.0001.).

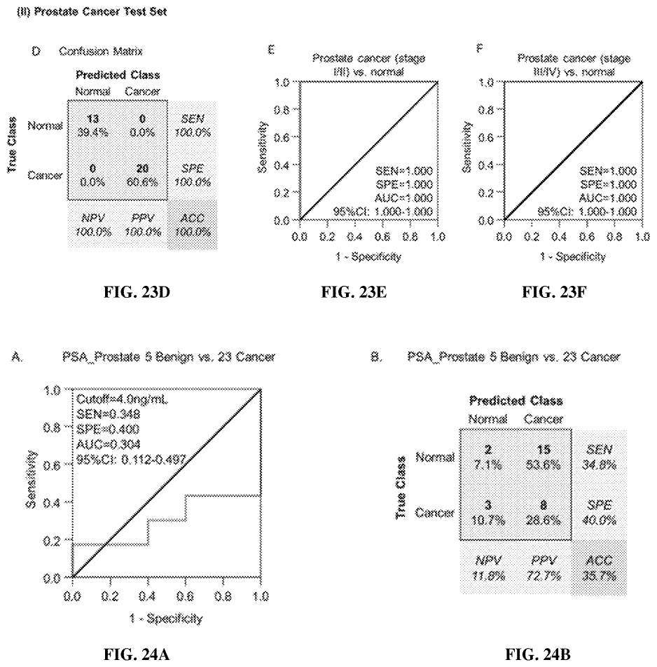

FIGS. 23A-23F show evaluation of exosomal TF-Ag-α for prostate cancer diagnosis using a training set and an independent test set. FIG. 23A is a confusion matrix showing the diagnostic performance of exosomal TF-Ag-α in prostate cancer based on the training set which included 40 prostate cancer patients (stage I/II: n=20; stage III/IV: n=20) and 30 normal controls (low risk: n=10; benign conditions at high risk of cancer: n=20). FIG. 23B shows ROC curve analysis comparing early-stage prostate cancer patients (Stage I/II, n=20) vs. normal controls (n=30) in the training set. FIG. 23C shows ROC curve analysis comparing late-stage prostate cancer patients (Stage III/IV, n=20) vs. normal controls (n=30) in the training set. FIG. 23D is a confusion matrix showing the diagnostic performance of exosomal TF-Ag-α based on the test set which included 20 prostate cancer patients (stage I/II: n=10; stage III/IV: n=10) and 13 normal controls. FIG. 23E shows ROC curve analysis comparing early-stage prostate cancer patients (Stage I/II, n=10) vs. normal controls (n=13) in the test set. FIG. 23F shows ROC curve analysis comparing late-stage prostate cancer patients (Stage III/IV, n=10) vs. normal controls (n=13) in the test set. (SEN: sensitivity, SPE: specificity, NPV: negative predictive value; PPV: positive predictive value, ACC: overall accuracy, AUC: area under the curve.)

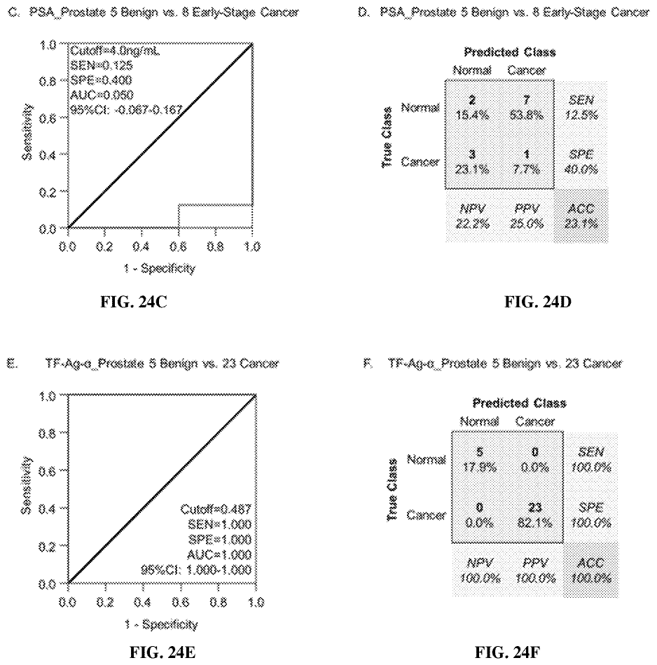

FIGS. 24A-24H show diagnostic performance comparison between PSA and exosomal TF-Ag-α in prostate cancer. Pre-treatment PSA data were available for a subset of prostate cancer patients (n=23) and patients with benign conditions (n=5), with blood samples collected within 100 days of those used for exosomal TF-Ag-α analysis to maintain temporal and analytical consistency. FIG. 24A shows ROC curve analysis comparing patients with prostate cancer (n=23, stage I-IV) vs. benign conditions (n=5) using PSA as the biomarker. FIG. 24B is a confusion matrix illustrating the diagnostic performance of PSA based on 23 prostate cancer patients and 5 patients with benign conditions. FIG. 24C shows ROC curve analysis comparing patients with early-stage prostate cancer (n=8, stage I-II) vs. benign conditions (n=5) using PSA as the biomarker. FIG. 24D is a confusion matrix showing diagnostic performance of PSA based on 8 early-stage prostate cancer patients and 8 patients with benign conditions. FIG. 24E shows ROC curve analysis comparing patients with prostate cancer (n=23, stage I-IV) vs. benign conditions (n=5) using exosomal TF-Ag-α as the biomarker. FIG. 24F is a confusion matrix showing diagnostic performance of exosomal TF-Ag-α based on 23 prostate cancer patients and 5 patients with benign conditions. FIG. 24G shows ROC curve analysis comparing patients with early-stage prostate cancer (n=8, stage I-II) vs. benign conditions (n=5) using exosomal TF-Ag-α as the biomarker. FIG. 24H is a confusion matrix showing diagnostic performance of exosomal TF-Ag-α based on 8 early-stage prostate cancer patients and 5 patients with benign conditions.

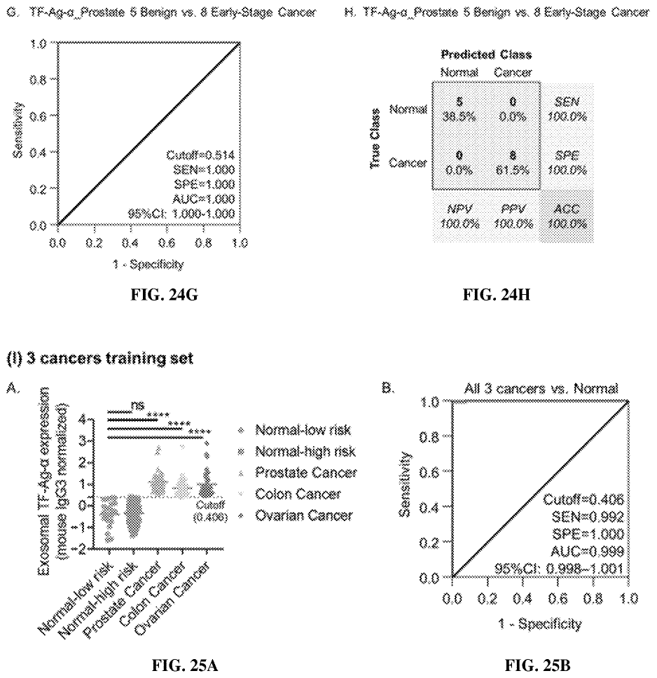

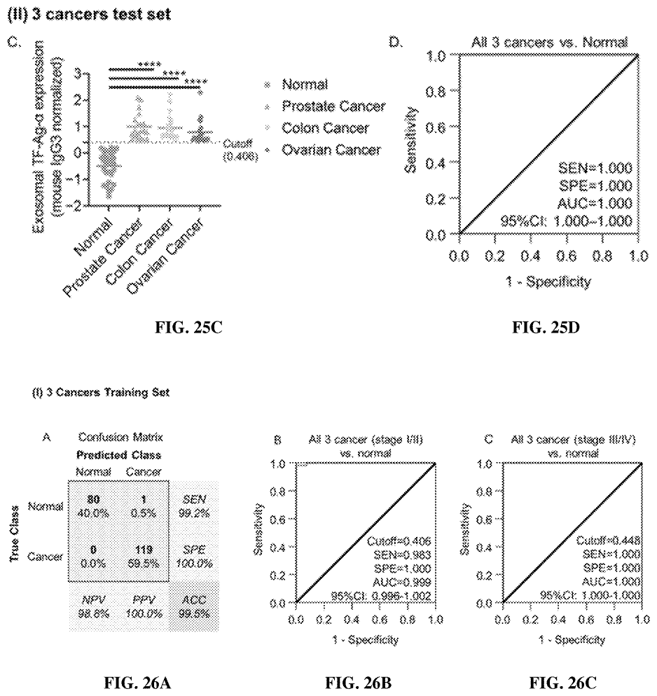

FIGS. 25A-25D show evaluation of exosomal TF-Ag-α in three cancers' diagnosis using the pooled training sets and independent test sets (colon, ovarian and prostate cancers). FIG. 25A is a scatterplot showing serum exosomal TF-Ag-α levels in three cancer patients (n=120; stage I/II: n=60; stage III/IV: n=60) and normal controls (n=80; low risk: n=20; high risk: n=60) in the training sets. FIG. 25B shows ROC curve analysis comparing all 3 cancer patients vs. normal controls in the training set. FIG. 25C is a scatterplot showing serum exosomal TF-Ag-α levels in three cancer patients (n=60; stage I/II: n=30; stage III/IV: n=30) and normal controls (n=29) in the test sets. FIG. 25D shows ROC curve analysis comparing all three cancer patients vs. normal controls in the test sets. (SEN: sensitivity, SPE: specificity, AUC: area under the curve. ns: not significant; ****: p<0.0001).

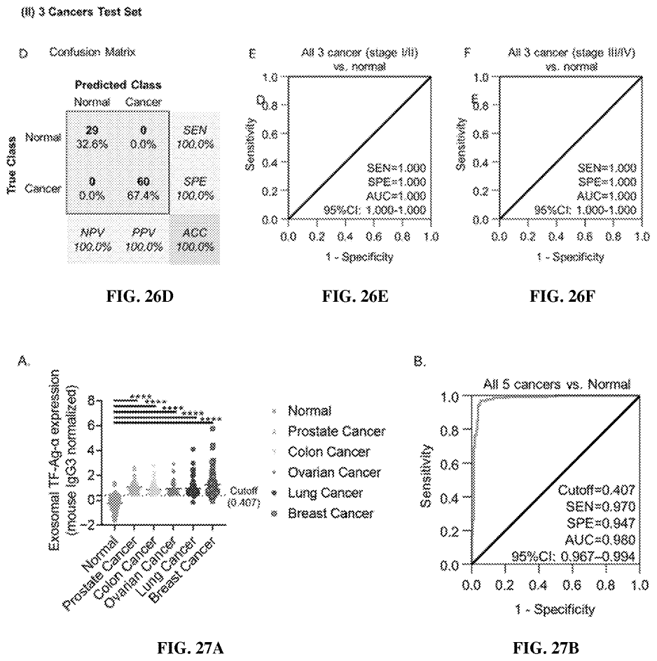

FIGS. 26A-26F show evaluation of exosomal TF-Ag-α in three cancer diagnosis using the pooled training sets and independent test sets (colon, ovarian and prostate cancers). FIG. 26A is a confusion matrix showing the diagnostic performance of exosomal TF-Ag-α in 3 cancers based on the pooled training sets which included 120 cancer patients (stage I/II: n=60; stage III/IV: n=60) and 80 normal controls (low risk: n=20; benign conditions at high risk of cancer: n=60). FIG. 26B shows ROC curve analysis comparing early-stage cancer patients (Stage I/II, n=60) vs. normal controls (n=80) in the training set. FIG. 26C shows ROC curve analysis comparing late-stage cancer patients (Stage III/IV, n=60) vs. normal controls (n=80) in the training set. FIG. 26D is a confusion matrix showing the diagnostic performance of exosomal TF-Ag-α based on the pooled test sets which included 60 cancer patients (stage I/II: n=30; stage III/IV: n=30) and 29 normal controls. FIG. 26E shows ROC curve analysis comparing early-stage cancer patients (Stage I/II, n=30) vs. normal controls (n=29) in the test set. FIG. 26F shows ROC curve analysis comparing late-stage cancer patients (Stage III/IV, n=30) vs. normal controls (n=29) in the test set. (SEN: sensitivity, SPE: specificity, NPV: negative predictive value; PPV: positive predictive value, ACC: overall accuracy, AUC: area under the curve.)

FIGS. 27A-27B show evaluation of exosomal TF-Ag-α in 5 cancer diagnosis (colon, ovarian, prostate, lung and breast cancers). FIG. 27A is a scatterplot showing serum exosomal TF-Ag-α levels in patients with five cancers (n=335; stage 0/I/II: n=174; stage III/IV: n=161) and normal controls (n=187; low risk: n=89; high risk: n=98). FIG. 27B shows ROC curve analysis comparing all cancer patients vs. normal controls. (SEN: sensitivity, SPE: specificity, AUC: area under the curve. ns: not significant; ****: p<0.0001).

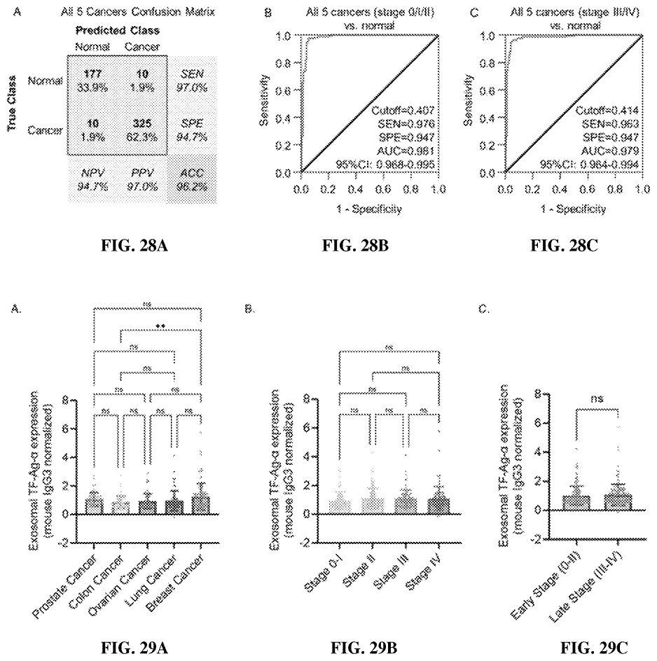

FIGS. 28A-28C show evaluation of exosomal TF-Ag-α in five cancer diagnosis (colon, ovarian, prostate, lung and breast cancers). FIG. 28A is a confusion matrix showing the diagnostic performance of exosomal TF-Ag-α in 5 cancers based on all patient data which included 335 cancer patients (stage 0/1/II: n=174; stage III/IV: n=161) and 187 normal controls (low risk: n=89; benign conditions at high risk of cancer: n=98). FIG. 28B shows ROC curve analysis comparing early-stage cancer patients (Stage I/II, n=174) vs. normal controls (n=187). FIG. 28C shows ROC curve analysis comparing late-stage cancer patients (Stage III/IV, n=161) vs. normal controls (n=187). (SEN: sensitivity, SPE: specificity, NPV: negative predictive value; PPV: positive predictive value, ACC: overall accuracy, AUC: area under the curve.)

FIGS. 29A-29C show exosomal TF-Ag-α levels remain largely consistent across cancer types and stages. One-way ANOVA analysis of exosomal TF-Ag-α levels across cancer types and stages in five cancer types (colon, ovarian, prostate, lung and breast). FIG. 29A shows Brown-Forsythe and Welch-corrected one-way ANOVA revealed significant differences in exosomal TF-Ag-α levels among the five cancer types (F (4.000, 160.9)=3.728, p=0.0063, **; F*(4.000, 286.8)=4.381, p=0.0019, **). Post-hoc Games-Howell comparisons indicated that breast cancer patients had significantly higher exosomal TF-Ag-α levels than colon cancer patients (p=0.0071, **), while all other pairwise differences were not significant (ns). FIG. 29B shows Brown-Forsythe and Welch-corrected one-way ANOVA showed no significant differences in exosomal TF-Ag-α levels among cancer stages (F (3.000, 177.6)=0.8260, p=0.4811; F*(3.000, 289.7)=0.6793, p=0.5653). Post-hoc Games-Howell comparisons showed no significant pairwise differences. FIG. 29C shows Welch's unpaired t-test comparing early-stage (0-II) and late-stage (III-IV) cancers showed no significant difference in exosomal TF-Ag-α levels (P=0.4344, ns).

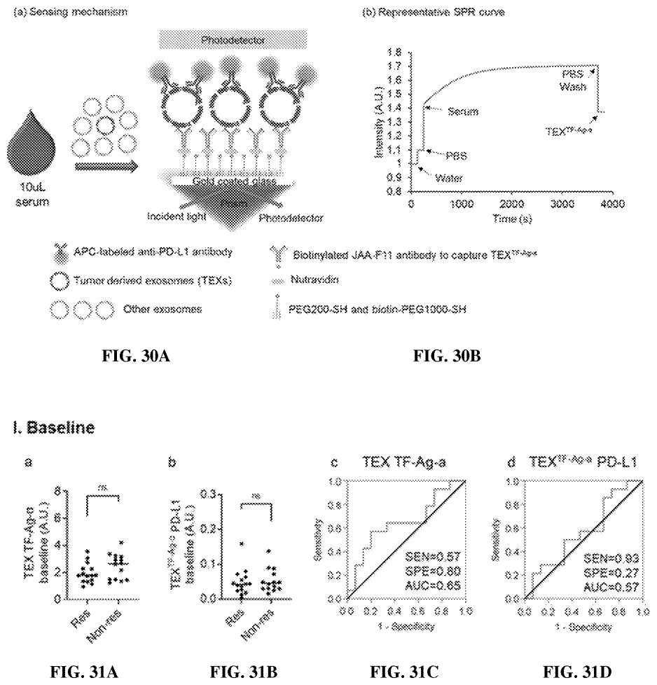

FIGS. 30A-30B show detection of TEX^{TF-Ag-α} PD-L1 using Exo-PROS assay. FIG. 30A shows the sensing mechanism of Exo-PROS assay. FIG. 30B shows a representative SPR curve for detecting TEX TF-Ag-α in a stage IV NSCLC patient serum sample.

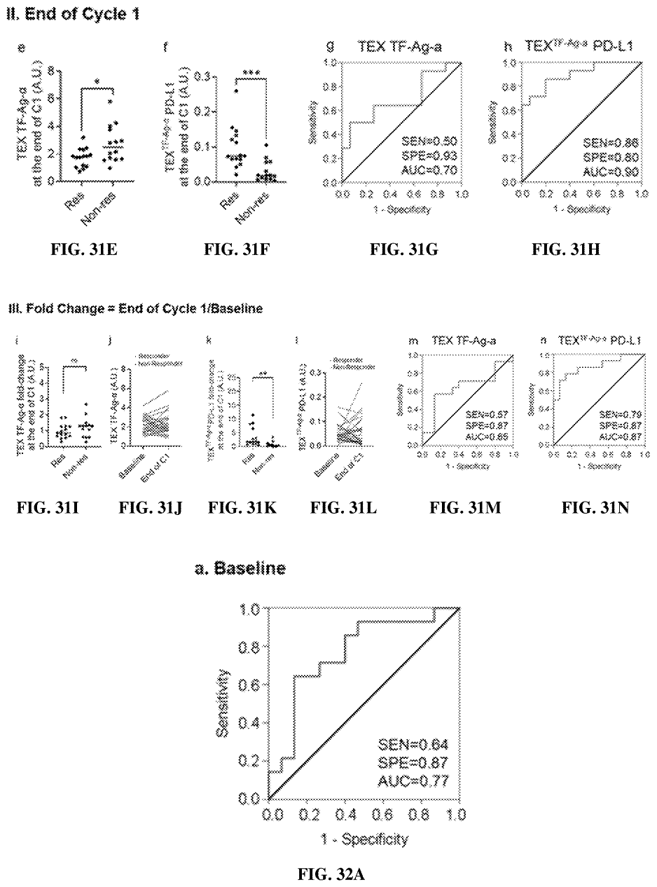

FIGS. 31A-31N show evaluation of TEX TF-Ag-α and TEX^{TF-Ag-α} PD-L1 for predicting response to anti-PD-1 therapy in a discovery cohort of NSCLC patients (n=15 responders; n=14 non-responders). FIGS. 31A-31B show TEX TF-Ag-α expression (FIG. 31A) and TEX^{TF-Ag-α} PD-L1 (FIG. 31B) expression at pre-treatment baseline. FIGS. 31C-31D show ROC analysis of the predictive performance of baseline TEX TF-Ag-α (FIG. 31C) and baseline TEX^{TF-Ag-α} PD-L1 (FIG. 31D) for distinguishing responders from non-responders. FIGS. 31E-31F show TEX TF-Ag-α expression (FIG. 31E) and TEX^{TF-Ag-α} PD-L1 expression (FIG. 31F) at the end of first treatment cycle. FIGS. 31G-31H show ROC analysis of the predictive performance of end-of-cycle 1 TEX TF-Ag-α (FIG. 31G) and end-of-cycle 1 TEX^{TF-Ag-α} PD-L1 (FIG. 31H) for distinguishing responders from non-responders. FIGS. 31I-31L show TEX TF-Ag-α expression fold change (FIGS. 31I-31J) and TEX_{TF-Ag-α} PD-L1 expression fold change (FIGS. 31K-31L) between the end of treatment cycle 1 and the pre-treatment baseline. FIGS. 31M-31N show ROC analysis of the predictive performance of TEX TF-Ag-α fold change (FIG. 31M) and TEX^{TF-Ag-α} PD-L1 fold change (FIG. 31N) for distinguishing responders from non-responders.

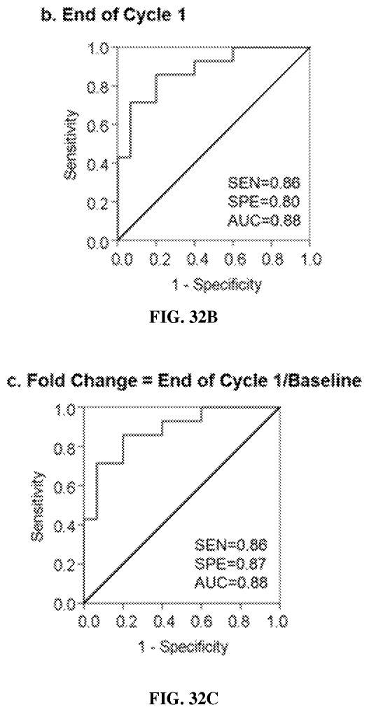

FIGS. 32A-32C show evaluation of TEX TF-Ag-α and TEX^{TF-Ag-α} PD-L1 combined biomarkers for predicting response to anti-PD-1 therapy in a discovery cohort of NSCLC patients (n=15 responders; n=14 non-responders). FIGS. 32A-32C show ROC analysis of the predictive performance of the combination of two baseline-based biomarkers (FIG. 32A), two end-of-cycle 1-based biomarkers (FIG. 32B), and two fold-change-based biomarkers (FIG. 32C) for distinguishing responders from non-responders.

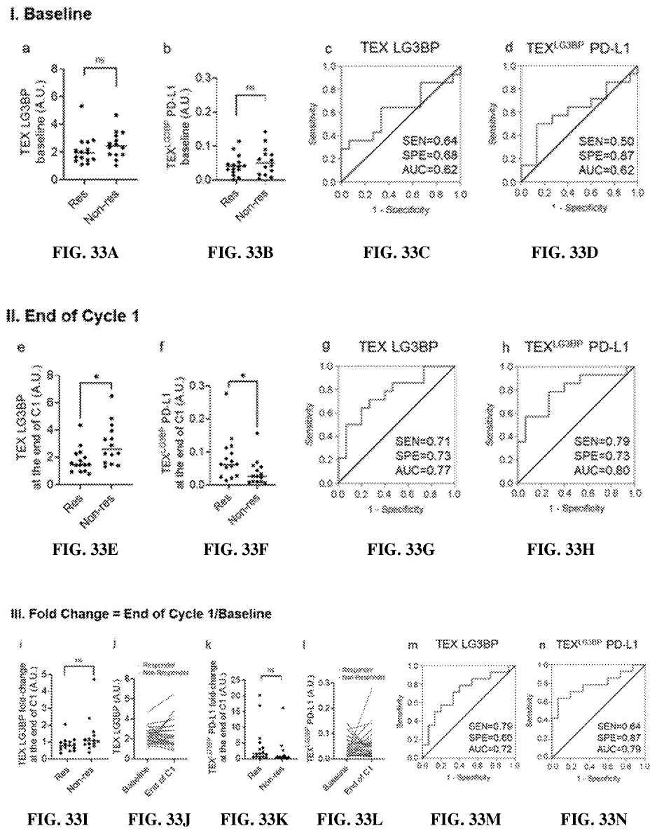

FIGS. 33A-33N show evaluation of TEX LG3BP and TEX^{LG3BP} PD-L1 for predicting response to anti-PD-1 therapy in a discovery cohort of NSCLC patients (n=15 responders; n=14 non-responders). FIGS. 33A-33B show TEX LG3BP expression (FIG. 33A) and TEX^{LG3BP} PD-L1 expression (FIG. 33B) at pre-treatment baseline. FIGS. 33C-33D show ROC analysis of the predictive performance of baseline TEX LG3BP (FIG. 33C) and baseline TEX_{LG3BP} PD-L1 (FIG. 33D) for distinguishing responders from non-responders. FIGS. 33E-33F show TEX LG3BP expression (FIG. 33E) and TEX^{LG3BP} PD-L1 expression (FIG. 33F) at the end of first treatment cycle. FIGS. 33G-33H show ROC analysis of the predictive performance of end-of-cycle 1 TEX LG3BP (FIG. 33G) and end-of-cycle 1 TEX^{LG3BP} PD-L1 (FIG. 33H) for distinguishing responders from non-responders. FIGS. 33I-33L show TEX LG3BP expression fold change (FIGS. 33I-33J) and TEX^{LG3BP} PD-L1 expression fold change (FIGS. 33K-33L) between the end of treatment cycle 1 and the pre-treatment baseline. FIGS. 33M-33N show ROC analysis of the predictive performance of TEX LG3BP fold change (FIG. 33M) and TEX^{LG3BP} PD-L1 fold change (FIG. 33N) for distinguishing responders from non-responders.

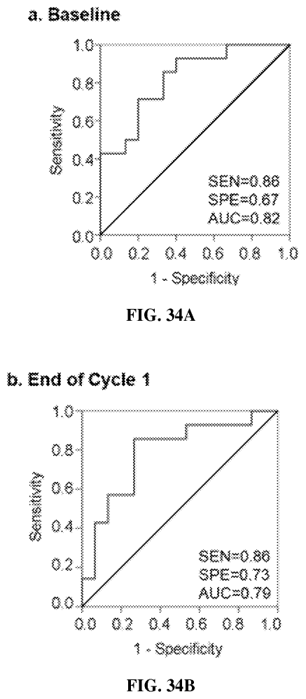

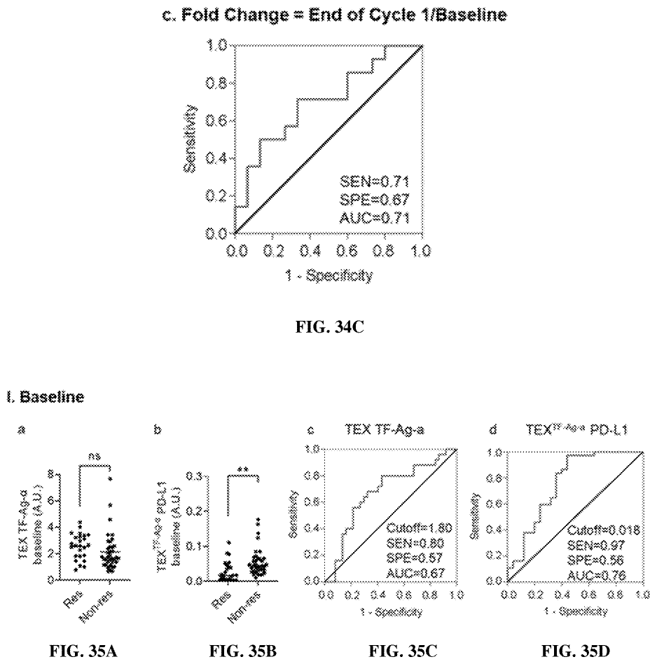

FIGS. 34A-34C show evaluation of TEX LG3BP and TEX^{LG3BP} PD-L1 combined biomarkers for predicting response to anti-PD-1 therapy in a discovery cohort of NSCLC patients (n=15 responders; n=14 non-responders). FIGS. 34A-34C show ROC analysis of the predictive performance of the combination of two baseline-based biomarkers (FIG. 34A), two end-of-cycle 1-based biomarkers (FIG. 34B), and two fold-change-based biomarkers (FIG. 34C) for distinguishing responders from non-responders.

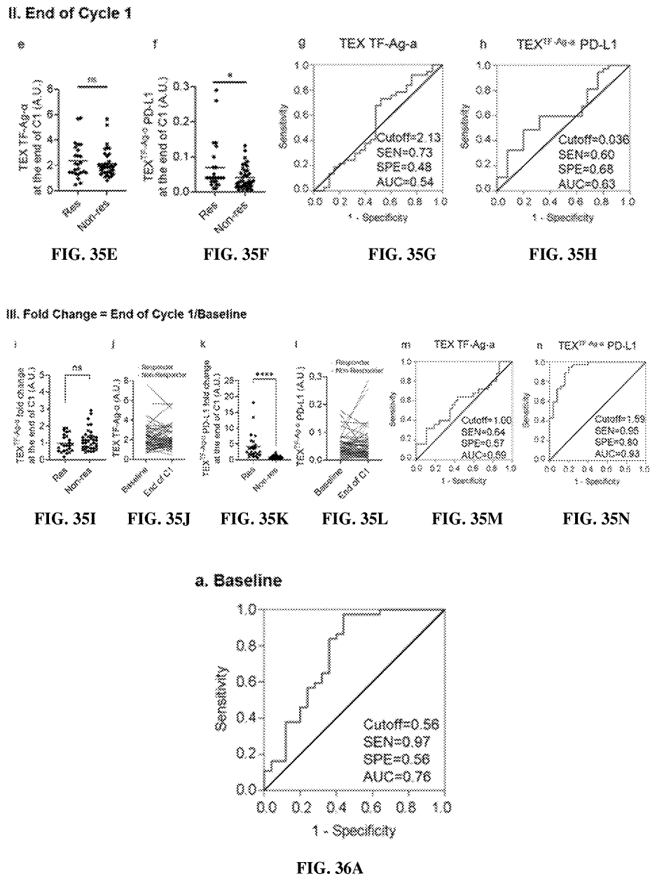

FIGS. 35A-35N show evaluation of TEX TF-Ag-α and TEX^{TF-Ag-α} PD-L1 for predicting response to anti-PD-1 therapy in a validation cohort of NSCLC patients (n=25 responders; n=37 non-responders). FIGS. 35A-35B show TEX TF-Ag-α expression (FIG. 35A) and TEX^{TF-Ag-α} PD-L1 expression (FIG. 35B) at pre-treatment baseline. FIGS. 35C-35D show ROC analysis of the predictive performance of baseline TEX TF-Ag-α (FIG. 35C) and baseline TEX^{TF-Ag-α} PD-L1 (FIG. 35D) for distinguishing responders from non-responders. FIGS. 35E-35F show TEX TF-Ag-α expression (FIG. 35E) and TEX^{TF-Ag-α} PD-L1 expression (FIG. 35F) at the end of first treatment cycle. FIGS. 35G-35H show ROC analysis of the predictive performance of end-of-cycle 1 TEX TF-Ag-α (FIG. 35G) and end-of-cycle 1 TEX^{TF-Ag-α} PD-L1 (FIG. 35H) for distinguishing responders from non-responders. FIGS. 35I-35L show TEX TF-Ag-α expression fold change (FIGS. 35I-35J) and TEX_{TF-Ag-α} PD-L1 expression fold change (FIGS. 35K-35L) between the end of treatment cycle 1 and the pre-treatment baseline. FIGS. 35M-35N show ROC analysis of the predictive performance of TEX TF-Ag-α fold change (FIG. 35M) and TEX^{TF-Ag-α} PD-L1 fold change (FIG. 35N) for distinguishing responders from non-responders.

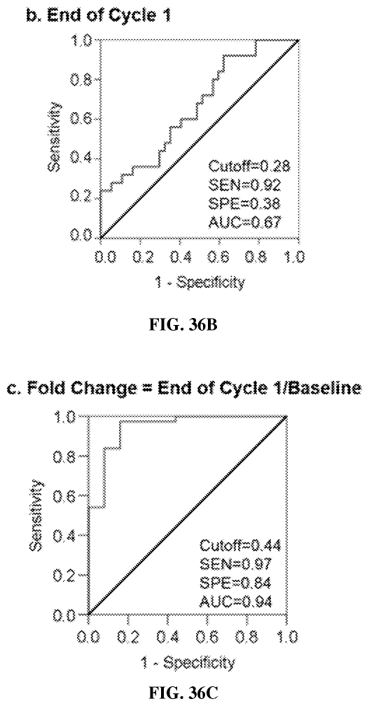

FIGS. 36A-36C show evaluation of TEX TF-Ag-α and TEX^{TF-Ag-α} PD-L1 combined biomarkers for predicting response to anti-PD-1 therapy in a validation cohort of NSCLC patients (n=25 responders; n=37 non-responders). FIGS. 36A-36C show ROC analysis of the predictive performance of the combination of two baseline-based biomarkers (FIG. 36A), two end-of-cycle 1-based biomarkers (FIG. 36B), and two fold-change-based biomarkers (FIG. 36C) for distinguishing responders from non-responders.

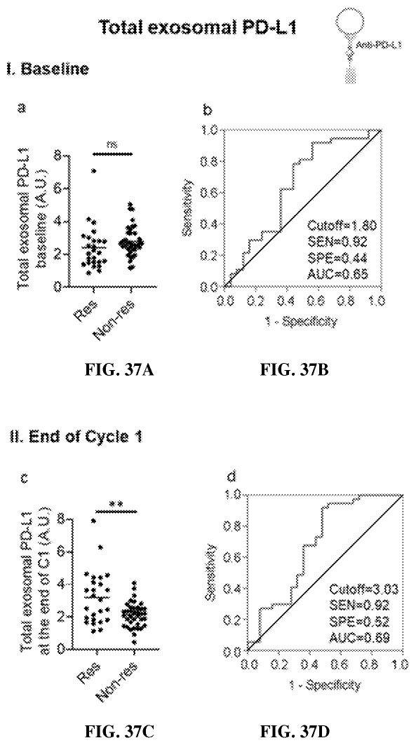

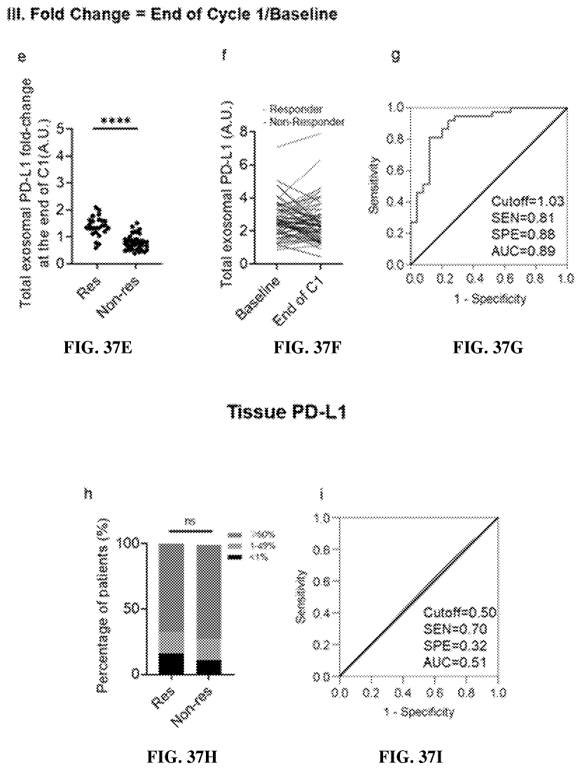

FIGS. 37A-37G show predictive performance of total exosomal PD-L1 and tissue PD-L1 for response to anti-PD-1 immunotherapy (n=25 responders; n=37 non-responders). Total exosomal PD-L1 expression is shown at the pre-treatment baseline (FIG. 37A), at the end of first treatment cycle (FIG. 37C), and as fold change (FIGS. 37E-37F). Corresponding ROC analyses for distinguishing responders from non-responders are shown in FIG. 37B, FIG. 37D, and FIG. 37G, respectively. FIGS. 37H-37I show tissue PD-L1 tumor proportion score (TPS, FIG. 37H) and corresponding ROC analysis (FIG. 37I) for distinguishing responders from non-responders.

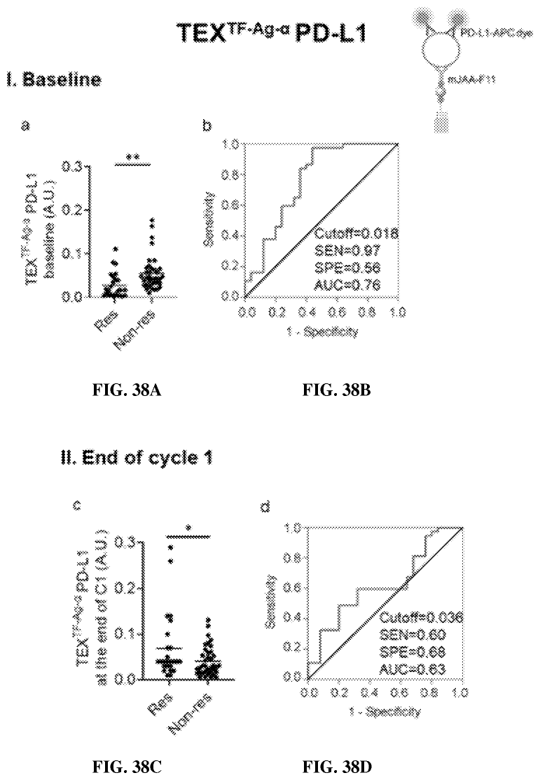

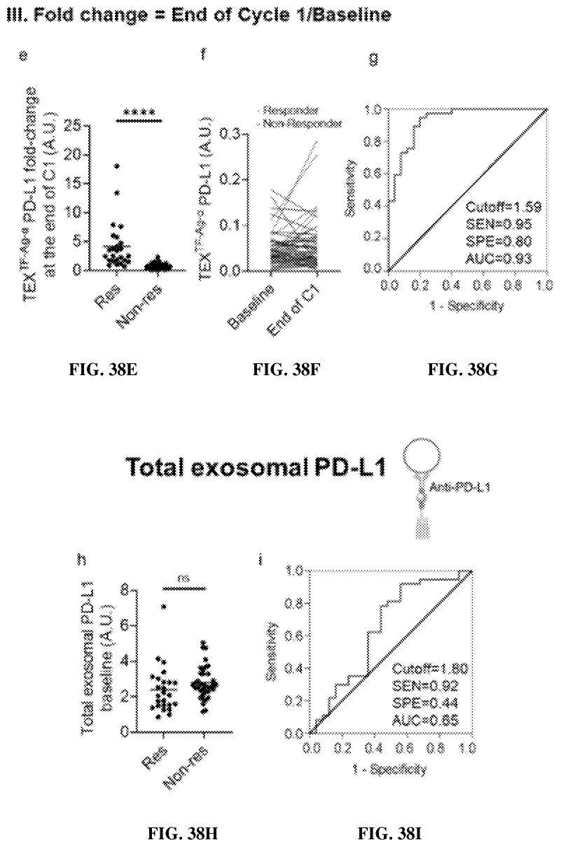

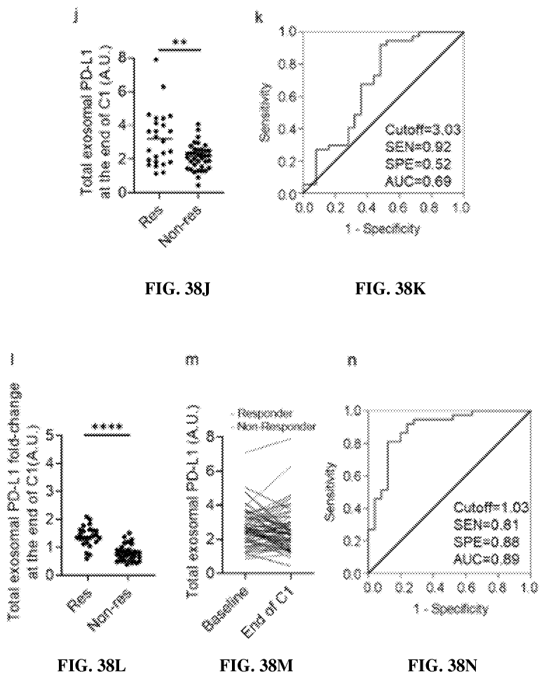

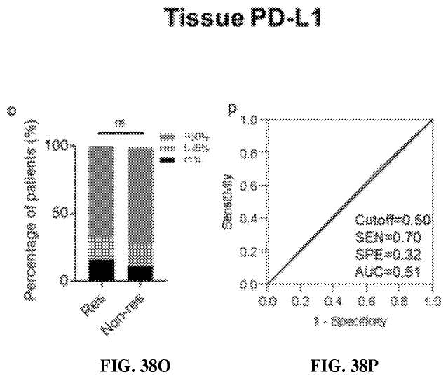

FIGS. 38A-38P show comparison of predictive performance of TEX^{TF-Ag-α} PD-L1, total exosomal PD-L1 and tissue PD-L1 for response to anti-PD-1 immunotherapy in the validation cohort (n=25 responders; n=37 non-responders). TEX^{TF-Ag-α} PD-L1 expression is shown at the pre-treatment baseline (FIG. 38A), at the end of first treatment cycle (FIG. 38C), and as fold change (FIGS. 38E-38F). Corresponding ROC analyses for distinguishing responders from non-responders are shown in FIG. 38B, FIG. 38D, and FIG. 38G, respectively. Total exosomal PD-L1 expression is shown at the pre-treatment baseline (FIG. 38H), at the end of first treatment cycle (FIG. 38J) and as fold change (FIGS. 38L-38M). Corresponding ROC analyses for distinguishing responders from non-responders are shown in FIG. 38I, FIG. 38K, and FIG. 38N, respectively. FIGS. 38O-38P show tissue PD-L1 tumor proportion score (TPS, FIG. 38O) and corresponding ROC analysis (FIG. 38P) for distinguishing responders from non-responders.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “subject” is defined herein to include animals such as mammals, including, but not limited to, primates (e.g., humans), cows, horses, pigs, sheep, goats, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “decrease” can refer to any change that results in a smaller gene expression, protein production, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain (i.e., nociception) relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Tumor-derived exosomes (TEX) play important roles in every step of cancer development, immune regulation, metastasis and drug resistance, therefore, TEX are promising biomarkers for cancer liquid biopsy. Recent studies have shown that exosomal PD-L1 is closely correlated with response to ICI therapy. However, exosomes expressing PD-L1 are released not only from tumor cells but also by immune cells, such as T cells, B cells, NK cells, dendritic cells, macrophages. Selective capture of TEX followed by the detection of TEX PD-L1 represents a new strategy to achieve high predictive sensitivity, specificity and accuracy. Accordingly, disclosed herein are methods and systems to detect expression level of a TEX biomarker in a subject using liquid biopsy.

Exosomes are nanosized extracellular vesicles released by cells to transport biomolecules such as proteins and RNAs for intercellular communication. Exosomes play important roles in cancer development and metastasis; therefore, they have emerged as potential liquid biopsy biomarkers for cancer screening, diagnosis and management. Many exosome cargos, including proteins, RNAs and lipids, have been extensively investigated as biomarkers for cancer liquid biopsy. However, carbohydrates, an important type of biomolecules, have not yet been explored for this purpose. In this study, a new exosomal carbohydrate biomarker is reported, i.e., alpha-linked Thomsen-Friedenreich glycoantigen (TF-Ag-α; Galβ1-3GalNAc alpha). To transform the discovery for clinical applications, a SPR-based assay is introduced which utilizes a unique monoclonal antibody, JAA-F11, with high specificity to TF-Ag-α to measure the levels of exosomal TF-Ag-α in blood. Exosomal TF-Ag-α was found in as low as 10 μL serum samples from cancer patients but in contrast, levels were negligible from normal controls. It is shown that exosomal TF-Ag-α detected lung cancer and breast cancer from normal controls with ≥95% and ≥97% accuracy, respectively. These results demonstrate that exosomal TF-Ag-α is a potential liquid biopsy biomarker for cancer diagnosis.

TEX carry various biomolecules including nucleic acids, proteins and lipids from their parental cells. and actively participate in tumor initiation, development and metastasis. Therefore, TEX, especially their RNA and protein cargos, are promising liquid biopsy biomarkers for cancer screening and diagnosis.

In one aspect, disclosed herein are methods of detecting a TEX biomarker (including, but not limited to exosomes derived from lung cancer, breast cancer, prostate cancer, colon cancer, ovarian cancer, or bladder cancer) in a subject using liquid biopsy (including, but not limited to cerebrospinal fluid, saliva, amniotic fluid, ascites, blood, breast milk, bone marrow, or urine) comprising obtaining a liquid biopsy sample from the subject; capturing TEX in the liquid biopsy sample using an antibody (such as, for example JAA-F11) specific to the TEX biomarker (such as, for example TF-Ag-α); detecting SPR signal generated by the capture of TEX; measuring the expression of TEX biomarker based on SPR signal; analyzing the signal between the sample with TEX biomarker and reference sample, wherein the reference sample is obtained from a healthy subject; and administering a therapeutically effective dose of a humanized TEX biomarker specific antibody to the subject with a higher TEX biomarker expression compared to the reference sample from a healthy subject. For example, in one aspect, disclosed herein are methods for determining a TEX biomarker in a subject comprising: obtaining a sample from the subject via liquid biopsy (including, but not limited to liquid biopsy of cerebrospinal fluid, saliva, amniotic fluid, ascites, blood, breast milk, bone marrow, or urine); using an antibody to TF-Ag-α to capture the TEX in the sample; using SPR to measure the levels of TF-Ag-α on captured TEX; using a second fluorescence labeled antibody that binds to second TEX biomarker (for example, a marker associated with a therapy, including, but not limited to PD-L1); using surface plasmon enhanced fluorescence (SPEF) spectrometry to measure the expression of second TEX marker; analyzing the signal between the sample with TF-Ag and the second TEX biomarker and the reference sample, wherein the reference sample is obtained from a healthy subject; determining the TEX biomarker expression; administering a therapeutically effective dose of a humanized TEX biomarker specific antibody to the subject with a different TEX biomarker expression compared to the reference sample from a healthy subject.

For example, disclosed herein are methods for determining a TEX biomarker in a subject comprising: obtaining a sample from the subject via liquid biopsy (including, but not limited to liquid biopsy of cerebrospinal fluid, saliva, amniotic fluid, ascites, blood, breast milk, bone marrow, or urine); capturing TEX using JAA-F11 antibody to TF-Ag-α in the sample; using SPR to measure the levels of TF-Ag-α on captured TEX; using a second fluorescence labeled antibody that binds to second TEX biomarker (for example, a marker associated with a therapy, including, but not limited to PD-L1); using surface plasmon enhanced fluorescence (SPEF) spectrometry to measure the expression of second TEX marker (for example, TEX PD-L1); analyzing the signal between the sample with TF-Ag and the second TEX biomarker and the reference sample, wherein the reference sample is obtained from a healthy subject; determining the TEX biomarker expression; administering a therapeutically effective dose of a humanized TEX biomarker specific antibody if the subject has a different TEX biomarker expression compared to the reference sample from a healthy subject.

Also disclosed herein in one aspect are methods of detecting a TEX biomarker, wherein the biochip further enables multiplex detection of additional TEX biomarkers co-expressed on TEX, wherein the additional TEX biomarkers comprise PD-L1, PD-1, LAG-3, TIM-3, or CTLA-4.

In one aspect are methods of detecting a TEX biomarker, wherein the expression level of the TF-Ag-α in the sample is calculated using following equation:

$$Expression of TEX TF - Ag - α = {( \frac{{I}_{TEX TF - Ag - α} - {I}_{PBS}}{{I}_{PBS} - {I}_{water}} )}_{JAA - F 11 antibody modified biochip} - {( \frac{{I}_{TEX TF - Ag - α} - {I}_{PBS}}{{I}_{PBS} - {I}_{water}} )}_{IgG 3 control antibody modified biochip}$$

wherein water and PBS emit baseline signal; wherein JAA-F11 is an antibody against TF-Ag-α; wherein IgG3 is a control antibody; wherein a higher expression level of TEX TF-Ag-α in the sample relative to the reference sample indicates presence of a cancer.

Also described herein are systems to detect expression level of a TEX biomarker (including, but not limited to exosomes derived from lung cancer, breast cancer, prostate cancer, colon cancer, ovarian cancer, or bladder cancer) in a subject using liquid biopsy (including, but not limited to cerebrospinal fluid, saliva, amniotic fluid, ascites, blood, breast milk, bone marrow, or urine), comprising: a biochip for detection of TEX biomarker (such as, for example, a protein, a nucleic acid, a carbohydrate or a lipid including, but not limited to TF-Ag-α) in a liquid biopsy sample; an antibody (such as, for example JAA-F11) immobilized on the biochip, wherein the antibody is specific to the TEX biomarker; a biosensor (photodetector) to record a SPR signal from the liquid biopsy sample; wherein the SPR signal is obtained when TEX biomarker binds to the antibody; and an equation to analyze expression level of the TEX biomarker, wherein a high TEX biomarker expression compared to a reference sample indicates presence of a cancer.

Disclosed herein, in one aspect are methods and systems wherein the expression level of the TF-Ag-α in the sample is calculated using the equation:

$$Expression of TEX TF - Ag - α = {( \frac{{I}_{TEX TF - Ag - α} - {I}_{PBS}}{{I}_{PBS} - {I}_{water}} )}_{JAA - F 11 antibody modified biochip} - {( \frac{{I}_{TEX TF - Ag - α} - {I}_{PBS}}{{I}_{PBS} - {I}_{water}} )}_{IgG 3 control antibody modified biochip}$$

wherein water and PBS emit baseline signal; wherein JAA-F11 is an antibody against TF-Ag-α; wherein IgG3 is a control antibody; wherein a higher expression level of the TF-Ag-α in the sample relative to the reference sample indicates presence of a cancer and the successful capture of TEX.

The clinical applications of many exosome components including nucleic acids (DNAs, mRNAs, microRNAs, lncRNAs, circular RNAs), proteins and lipids in cancer diagnosis have been extensively studied. However, carbohydrates, an important type of biomolecule, have not yet been investigated. In this study, a new exosomal marker that is carbohydrate-based is discovered, specifically the Thomsen-Friedenreich glycoantigen (TF-Ag-α; Galβ1-3GalNAc alpha). It is shown that exosomal TF-Ag-α is highly sensitive and specific for cancer diagnosis. Expression of the Thomsen-Friedenreich antigen (TF-Ag or CD176) on erythrocytes after exposure to bacterial neuraminidase was first described by Thomsen in 1927 and further detailed by Freidenreich. TF-Ag is a typically cryptic antigen that is aberrantly ‘unmasked’ or expressed in cancer cells due to incomplete surface glycosylation. It is an oncofetal antigen that has limited expression in normal adult tissues (e.g., placenta). There are two types of linkage configurations for TF-Ag, one is linked in alpha (α) orientation (expressed in cancer cells only) and the other is linked in beta (β) orientation (widely expressed in normal cells). TF-Ag-α plays important roles in tumor growth, immune evasion, and metastasis. Interestingly, immune response to TF-Ag-α correlate with cancer prognosis and survival. Thus, as noted above, disclosed herein are methods and systems for detecting expression level of TEX biomarkers using liquid biopsy, wherein the tumor-derived exosomal biomarker comprises a protein, a nucleic acid, a carbohydrate or a lipid, wherein the nucleic acid comprises a DNA, an mRNA, a microRNA, a lncRNA or a circular RNA. In one aspect, the carbohydrate is alpha-linked Thomsen-Friedenreich glycoantigen (TF-Ag-α; Galβ1-3GalNAc alpha).

In one aspect, In one aspect, disclosed herein are methods to assess the efficacy of a cancer immunotherapy response in a liquid biopsy comprising: obtaining a liquid biopsy (including, but not limited to cerebrospinal fluid, saliva, amniotic fluid, ascites, blood, breast milk, bone marrow, or urine); capturing TEX using TEX biomarker specific antibody (first antibody) (such as, for example JAA-F11) attached to a biochip; detecting a TEX biomarker (such as, for example, TF-Ag-α) in the liquid biopsy by measuring SPR related signal using a SPR biosensor; measuring TEX biomarker expression; exposing the biochip to a second antibody specific to a biomarker associated with response to an immune checkpoint inhibitor (such as, for example, anti-PD-1, anti-PD-L1, anti-LAG3, anti-TIM3 or anti-CTLA-4) co-expressed on TEX; measuring an SPEF signal related to the biomarker associated with response to an immune checkpoint inhibitor (ICI); measuring an SPEF signal related to TEX biomarker associated with the response to ICI (TEX-ICI); and administering a therapeutically effective dose of an ICI to a subject with increased expression of the TEX biomarker relative to a subject with no or decreased expression of TEX. For example, disclosed herein, in one aspect is a method to determine cancer immunotherapy response comprising: obtaining a sample from a subject via liquid biopsy (including, but not limited to liquid biopsy of cerebrospinal fluid, saliva, amniotic fluid, ascites, blood, breast milk, bone marrow, or urine); using an antibody to TF-Ag-α attached to a biochip to capture TEX expressing TF-Ag-α (i.e., TEX^{TF-Ag-α}); using SPR to measure the levels of TF-Ag-α on captured TEX^{TF-Ag-α}; using fluorescence labeled antibodies to bind to biomarkers associated with response to immunotherapy on the captured TEX^{TF-Ag-α}; using surface plasmon enhanced fluorescence (SPEF) spectrometry to measure the levels of biomarker associated with immunotherapy response on TEX^{TF-Ag-α} (TEX^{TF-Ag-α}-ICI), wherein immune checkpoint inhibitors (ICI) are a type of cancer immunotherapy; analyzing the TEX^{TF-Ag-α}-ICI expression between the sample and a reference sample, wherein the reference sample is obtained from the same subject prior to treatment (pretreatment baseline) or at the end of prior treatment cycle; calculating expression of TEX^{TF-Ag-α}-ICI; and administering a therapeutically effective dose of the ICI to the subject with increased expression of TEX^{TF-Ag-α}-ICI in contrast to the subject with no change or decreased expression of TEX^{TF-Ag-α}-ICI. In one aspect, the ICI comprises anti-immune checkpoint proteins monoclonal antibodies, wherein the monoclonal antibodies are anti-PD-1, anti-PD-L1, anti-LAG3, anti-TIM3 or anti CTLA-4.

For example, disclosed herein is a method to assess the efficacy of a cancer immunotherapy response in a liquid biopsy comprising: obtaining a sample from a subject via liquid biopsy (including, but not limited to liquid biopsy of cerebrospinal fluid, saliva, amniotic fluid, ascites, blood, breast milk, bone marrow, or urine); capturing the TEX in the sample with the JAA-F11 antibody to TF-Ag-α attached to a biochip; using SPR to measure the levels of TF-Ag-α on captured TEX that express TF-Ag-α, i.e., TEX^{TF-Ag-α}; and using fluorescence labeled antibodies to bind to biomarkers associated with response to immunotherapy on the captured TEX^{TF-Ag-α}; using surface plasmon enhanced fluorescence (SPEF) spectrometry to measure the levels of biomarker associated with response to immunotherapy on TEX^{TF-Ag-α}, i.e., TEX^{TF-Ag-α}-ICI, wherein immune checkpoint inhibitors (ICI) are one type of immunotherapy; calculating expression of TEX^{TF-Ag-α}-ICI; analyzing the TEX^{TF-Ag-α}-ICI expression between the sample and a reference sample, wherein the reference sample is obtained from the same subject prior to treatment (pretreatment baseline) or at the end of prior treatment cycle; and administering a therapeutically effective dose of the ICI to the subject with increased expression of TEX^{TF-Ag-α}-ICI in contrast to the subject with no change or decreased expression of TEX^{TF-Ag-α}-ICI. In one aspect, the ICI comprises anti-immune checkpoint proteins monoclonal antibodies, wherein the monoclonal antibodies are anti-PD-1, anti-PD-L1, anti-LAG3, anti-TIM3 or anti CTLA-4.

Also disclosed herein, in the method of any preceding aspect wherein the biomarker associated with immune checkpoint proteins co-expressed on TEX^{TF-Ag-α}-ICI in the sample is calculated using following equation:

$$Expression of {TEX}^{TF - Ag - α} PD - L 1 = ( \frac{{I}_{after APC - PD - L 1} - {I}_{before APC - PD - L 1}}{{I}_{before APC - PD - L 1}} ) / ( Expression of TEX TF - Ag - α ) = ( \frac{{I}_{after APC - PD - L 1} - {I}_{before APC - PD - L 1}}{{I}_{before APC - PD - L 1}} ) / ( \frac{{I}_{TEX TF - Ag - α} - {I}_{PBS}}{{I}_{PBS} - {I}_{water}} )$$

wherein the water and the PBS emit baseline signal; wherein TF-Ag-α is a TEX biomarker; wherein TEX TF-Ag-α is the level of TF-Ag-α expressed on TEX; wherein TEX^{TF-Ag-α} is tumor-derived exosomes that express TF-Ag-α; wherein TEX^{TF-Ag-α} PD-L1 is the biomarker associated with immune checkpoint proteins co-expressed on TEX^{TF-Ag-α}; wherein the PD-L1 is a biomarker associated with the response to ICI treatment (for example, pembrolizumab), wherein the APC is a fluorescent dye labelling anti-PD-L1 antibody; wherein after APC-PD-L1 is fluorescence intensity of APC after incubating biochip with anti-APC-PD-L1 antibody; and wherein before APC-PD-L1 is fluorescence intensity of APC before incubating biochip with anti-APC-PD-L1 antibody. Other types of immunodetection can be used in this multi-step method of capturing tumor derived exosomes, quantitating them and following with a detection of prognosis related molecules.

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorimetric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically, fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the aspect include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

Labeling can be either direct or indirect. In direct labeling, the detecting antibody (the antibody for the molecule of interest) or detecting molecules (the molecule that can be bound by an antibody to the molecule of interest) include a label. Detection of the label indicates the presence of the detecting antibody or detecting molecules, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecules. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecules can have a label or signal-generating molecule or moiety. The additional molecule can be an antibody, which can thus be termed a secondary antibody. Binding a secondary antibody to the primary antibody can form a complex with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecules can also be or include one of a pair of molecules or moieties that can bind to each other, such as the biotin/avidin pair. In this mode, the detecting antibody or detecting molecules should include the other member of the pair.

Other modes of indirect labeling include the detection of primary immune complexes by a two-step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of a substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Capture assays are generally more useful for quantitatively evaluating the concentration of a specific protein, or antibody to a specific protein, in a sample.

Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Fluorescence labeling and detection methods are widely used. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, AZ), mass spectrometry (Intrinsic Bioprobes; Ciphergen, Fremont, CA), resonance light scattering (Genicon Sciences, San Diego, CA) and atomic force microscopy [BioForce Laboratories].

Capture arrays form the basis of diagnostic chips and arrays for expression profiling. They employ high affinity capture reagents, such as conventional antibodies, single domains, engineered scaffolds or peptides, to bind and detect specific target ligands in high throughput manner.

Antibody arrays have the required properties of specificity and acceptable background, and some are available commercially (BD Biosciences, San Jose, CA; Clontech, Mountain View, CA; BioRad; Sigma, St. Louis, MO). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; Biolnvent, Lund, Sweden; Affitech, Walnut Creek, CA; Biosite, San Diego, CA). In addition to the conventional antibodies, Fab and scFv fragments, single V-domains from camelids or engineered human equivalents (Domantis, Waltham, MA) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, MA) and ‘Anticalins’ based on the lipocalin structure (Pieris Proteolab, Freising-Weihenstephan, Germany). These can be used on capture arrays in a similar fashion to antibodies and may have advantages of robustness and ease of production.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

The disclosed compositions can be used in the diagnosis and prognosis of any disease where uncontrolled cellular proliferation occurs such as cancers and exosomes are representative of the cells. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

It is understood and herein contemplated that the disclosed detection and prognosis regimens can used alone or in combination with any anti-cancer therapy known in the art including, but not limited to Abemaciclib, Abiraterone Acetate, ABITREXATE® (Methotrexate), ABRAXANE® (Paclitaxel Albumin-stabilized Nanoparticle Formulation), ABVD, ABVE, ABVE-PC, AC, AC-T, ADCETRIS® (Brentuximab Vedotin), ADE, Ado-Trastuzumab Emtansine, ADRIAMYCIN® (Doxorubicin Hydrochloride), Afatinib Dimaleate, AFINITOR® (Everolimus), AKYNZEO® (Netupitant and Palonosetron Hydrochloride), ALDARA® (Imiquimod), Aldesleukin, ALECENSA® (Alectinib), Alectinib, Alemtuzumab, ALIMTA® (Pemetrexed Disodium), ALIQOPA® (Copanlisib Hydrochloride), ALKERAN™ for Injection (Melphalan Hydrochloride), ALKERAN™ Tablets (Melphalan), ALOXI® (Palonosetron Hydrochloride), ALUNBRIG® (Brigatinib), AMBOCHLORIN® (Chlorambucil), AMBOCLORIN® (Chlorambucil), Amifostine, Aminolevulinic Acid, Anastrozole, Aprepitant, AREDIA® (Pamidronate Disodium), ARIMIDEX® (Anastrozole), AROMASIN® (Exemestane), ARRANON® (Nelarabine), Arsenic Trioxide, ARZERRA® (Ofatumumab), Asparaginase Erwinia chrysanthemi, Atezolizumab, AVASTIN® (Bevacizumab), Avelumab, Axitinib, Azacitidine, BAVENCIO® (Avelumab), BEACOPP, BECENUM® (Carmustine), BELEODAQ® (Belinostat), Belinostat, Bendamustine Hydrochloride, BEP, BESPONSA® (Inotuzumab Ozogamicin), Bevacizumab, Bexarotene, BEXXAR® (Tositumomab and Iodine 1131 Tositumomab), Bicalutamide, BICNU® (Carmustine), Bleomycin, Blinatumomab, BLINCYTO® (Blinatumomab), Bortezomib, BOSULIF® (Bosutinib), Bosutinib, Brentuximab Vedotin, Brigatinib, BuMel, Busulfan, BUSULFEX® (Busulfan), Cabazitaxel, CABOMETYX® (Cabozantinib-S-Malate), Cabozantinib-S-Malate, CAF, CAMPATH® (Alemtuzumab), CAMPTOSAR® (Irinotecan Hydrochloride), Capecitabine, CAPOX, CARAC® (Fluorouracil-Topical), Carboplatin, CARBOPLATIN-TAXOL, Carfilzomib, CARMUBRIS® (Carmustine), Carmustine, Carmustine Implant, CASODEX® (Bicalutamide), CEM, Ceritinib, CERUBIDINE® (Daunorubicin Hydrochloride), CERVARIX® (Recombinant HPV Bivalent Vaccine), Cetuximab, CEV, Chlorambucil, CHLORAMBUCIL-PREDNISONE, CHOP, Cisplatin, Cladribine, CLAFEN® (Cyclophosphamide), Clofarabine, CLOFAREX® (Clofarabine), CLOLAR® (Clofarabine), CMF, Cobimetinib, COMETRIQ® (Cabozantinib-S-Malate), Copanlisib Hydrochloride, COPDAC, COPP, COPP-ABV, COSMEGEN® (Dactinomycin), COTELLIC® (Cobimetinib), Crizotinib, CVP, Cyclophosphamide, CYFOS® (Ifosfamide), CYRAMZA® (Ramucirumab), Cytarabine, Cytarabine Liposome, CYTOSAR-U® (Cytarabine), CYTOXAN® (Cyclophosphamide), Dabrafenib, Dacarbazine, DACOGEN® (Decitabine), Dactinomycin, Daratumumab, DARZALEX® (Daratumumab), Dasatinib, Daunorubicin Hydrochloride, Daunorubicin Hydrochloride and Cytarabine Liposome, Decitabine, Defibrotide Sodium, DEFITELIO® (Defibrotide Sodium), Degarelix, Denileukin Diftitox, Denosumab, DEPOCYT® (Cytarabine Liposome), Dexamethasone, Dexrazoxane Hydrochloride, Dinutuximab, Docetaxel, DOXIL® (Doxorubicin Hydrochloride Liposome), Doxorubicin Hydrochloride, Doxorubicin Hydrochloride Liposome, DOX-SL® (Doxorubicin Hydrochloride Liposome), DTIC-DOME® (Dacarbazine), Durvalumab, EFUDEX® (Fluorouracil-Topical), ELITEK® (Rasburicase), ELLENCE® (Epirubicin Hydrochloride), Elotuzumab, ELOXATIN® (Oxaliplatin), Eltrombopag Olamine, EMEND® (Aprepitant), EMPLICITI® (Elotuzumab), Enasidenib Mesylate, Enzalutamide, Epirubicin Hydrochloride, EPOCH, ERBITUX® (Cetuximab), Eribulin Mesylate, ERIVEDGE® (Vismodegib), Erlotinib Hydrochloride, ERWINAZE® (Asparaginase Erwinia chrysanthemi), ETHYOL® (Amifostine), Etopophos ETOPOPHOS® (Etoposide Phosphate), Etoposide, Etoposide Phosphate, EVACET® (Doxorubicin Hydrochloride Liposome), Everolimus, EVISTA® (Raloxifene Hydrochloride), EVOMELA® (Melphalan Hydrochloride), Exemestane, 5-FU® (Fluorouracil Injection), 5-FU® (Fluorouracil-Topical), FARESTON® (Toremifene), FARYDAK® (Panobinostat), FASLODEX® (Fulvestrant), FEC, FEMARA® (Letrozole), Filgrastim, FLUDARA® (Fludarabine Phosphate), Fludarabine Phosphate, FLUOROPLEX® (Fluorouracil-Topical), Fluorouracil Injection, Fluorouracil-Topical, Flutamide, FOLEX® (Methotrexate), FOLEX PFS® (Methotrexate), FOLFIRI, FOLFIRI-BEVACIZUMAB, FOLFIRI-CETUXIMAB, FOLFIRINOX, FOLFOX, FOLOTYN® (Pralatrexate), FU-LV, Fulvestrant, GARDASIL® (Recombinant HPV Quadrivalent Vaccine), GARDASIL 9® (Recombinant HPV Nonavalent Vaccine), GAZYVA® (Obinutuzumab), Gefitinib, Gemcitabine Hydrochloride, GEMCITABINE-CISPLATIN, GEMCITABINE-OXALIPLATIN, Gemtuzumab Ozogamicin, GEMZAR® (Gemcitabine Hydrochloride), GILOTRIF® (Afatinib Dimaleate), GLEEVEC® (Imatinib Mesylate), GLIADEL® (Carmustine Implant), GLIADEL WAFER® (Carmustine Implant), Glucarpidase, Goserelin Acetate, HALAVEN® (Eribulin Mesylate), HEMANGEOL® (Propranolol Hydrochloride), HERCEPTIN® (Trastuzumab), HPV Bivalent Vaccine, Recombinant, HPV Nonavalent Vaccine, Recombinant, HPV Quadrivalent Vaccine, Recombinant, HYCAMTIN® (Topotecan Hydrochloride), HYDREA® (Hydroxyurea), Hydroxyurea, Hyper-CVAD, IBRANCE® (Palbociclib), Ibritumomab Tiuxetan, Ibrutinib, ICE, ICLUSIG® (Ponatinib Hydrochloride), IDAMYCIN® (Idarubicin Hydrochloride), Idarubicin Hydrochloride, Idelalisib, IDHIFA® (Enasidenib Mesylate), IFEX® (Ifosfamide), Ifosfamide, IFOSFAMIDUM® (Ifosfamide), IL-2 (Aldesleukin), Imatinib Mesylate, IMBRUVICA® (Ibrutinib), IMFINZI® (Durvalumab), Imiquimod, IMLYGIC® (Talimogene Laherparepvec), INLYTA® (Axitinib), Inotuzumab Ozogamicin, Interferon Alfa-2b, Recombinant, Interleukin-2 (Aldesleukin), INTRON A® (Recombinant Interferon Alfa-2b), Iodine 1131 Tositumomab and Tositumomab, Ipilimumab, IRESSA® (Gefitinib), Irinotecan Hydrochloride, Irinotecan Hydrochloride Liposome, ISTODAX® (Romidepsin), Ixabepilone, Ixazomib Citrate, IXEMPRA® (Ixabepilone), JAKAFI® (Ruxolitinib Phosphate), JEB, JEVTANA® (Cabazitaxel), KADCYLA® (Ado-Trastuzumab Emtansine), KEOXIFENE® (Raloxifene Hydrochloride), KEPIVANCE® (Palifermin), KEYTRUDA® (Pembrolizumab), KISQALI® (Ribociclib), KYMRIAH® (Tisagenlecleucel), KYPROLIS® (Carfilzomib), Lanreotide Acetate, Lapatinib Ditosylate, LARTRUVO® (Olaratumab), Lenalidomide, Lenvatinib Mesylate, LENVIMA® (Lenvatinib Mesylate), Letrozole, Leucovorin Calcium, LEUKERAN® (Chlorambucil), Leuprolide Acetate, LEUSTATIN® (Cladribine), LEVULAN® (Aminolevulinic Acid), LINFOLIZIN® (Chlorambucil), LIPODOX® (Doxorubicin Hydrochloride Liposome), Lomustine, LONSURF® (Trifluridine and Tipiracil Hydrochloride), LUPRON® (Leuprolide Acetate), LUPRON DEPOT® (Leuprolide Acetate), LUPRON DEPOT-PED® (Leuprolide Acetate), LYNPARZA® (Olaparib), MARQIBO® (Vincristine Sulfate Liposome), MATULANE® (Procarbazine Hydrochloride), Mechlorethamine Hydrochloride, Megestrol Acetate, MEKINIST® (Trametinib), Melphalan, Melphalan Hydrochloride, Mercaptopurine, Mesna, MESNEX® (Mesna), METHAZOLASTONE® (Temozolomide), Methotrexate, METHOTREXATE LPF® (Methotrexate), Methylnaltrexone Bromide, MEXATE® (Methotrexate), MEXATE-AQ® (Methotrexate), Midostaurin, Mitomycin C, Mitoxantrone Hydrochloride, MITOZYTREX® (Mitomycin C), MOPP, MOZOBIL® (Plerixafor), MUSTARGEN® (Mechlorethamine Hydrochloride), MUTAMYCIN® (Mitomycin C), MYLERAN® (Busulfan), MYLOSAR® (Azacitidine), MYLOTARG® (Gemtuzumab Ozogamicin), NANOPARTICLE PACLITAXEL® (Paclitaxel Albumin-stabilized Nanoparticle Formulation), NAVELBINE® (Vinorelbine Tartrate), Necitumumab, Nelarabine, NEOSAR® (Cyclophosphamide), Neratinib Maleate, NERLYNX® (Neratinib Maleate), Netupitant and Palonosetron Hydrochloride, NEULASTA® (Pegfilgrastim), NEUPOGEN® (Filgrastim), NEXAVAR® (Sorafenib Tosylate), NILANDRON® (Nilutamide), Nilotinib, Nilutamide, NINLARO® (Ixazomib Citrate), Niraparib Tosylate Monohydrate, Nivolumab, NOLVADEX® (Tamoxifen Citrate), NPLATE® (Romiplostim), Obinutuzumab, ODOMZO® (Sonidegib), OEPA, Ofatumumab, OFF, Olaparib, Olaratumab, Omacetaxine Mepesuccinate, ONCASPAR® (Pegaspargase), Ondansetron Hydrochloride, ONIVYDE® (Irinotecan Hydrochloride Liposome), ONTAK® (Denileukin Diftitox), OPDIVO® (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, PARAPLAT® (Carboplatin), PARAPLATIN® (Carboplatin), Pazopanib Hydrochloride, PCV, PEB, Pegaspargase, Pegfilgrastim, Peginterferon Alfa-2b, PEG-INTRON® (Peginterferon Alfa-2b), Pembrolizumab, Pemetrexed Disodium, PERJETA® (Pertuzumab), Pertuzumab, PLATINOL® (Cisplatin), PLATINOL-AQ® (Cisplatin), Plerixafor, Pomalidomide, POMALYST® (Pomalidomide), Ponatinib Hydrochloride, PORTRAZZA® (Necitumumab), Pralatrexate, Prednisone, Procarbazine Hydrochloride, PROLEUKIN® (Aldesleukin), PROLIA® (Denosumab), PROMACTA® (Eltrombopag Olamine), Propranolol Hydrochloride, PROVENGE® (Sipuleucel-T), PURINETHOL® (Mercaptopurine), PURIXAN® (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, RELISTOR® (Methylnaltrexone Bromide), R-EPOCH, REVLIMID® (Lenalidomide), RHEUMATREX® (Methotrexate), Ribociclib, R-ICE, RITUXAN® (Rituximab), RITUXAN HYCELA® (Rituximab and Hyaluronidase Human), Rituximab, Rituximab and, Hyaluronidase Human, Rolapitant Hydrochloride, Romidepsin, Romiplostim, RUBIDOMYCIN® (Daunorubicin Hydrochloride), RUBRACA® (Rucaparib Camsylate), Rucaparib Camsylate, Ruxolitinib Phosphate, RYDAPT® (Midostaurin), Sclerosol Intrapleural Aerosol (Talc), Siltuximab, Sipuleucel-T, SOMATULINE DEPOT® (Lanreotide Acetate), Sonidegib, Sorafenib Tosylate, SPRYCEL® (Dasatinib), STANFORD V, Sterile Talc Powder (Talc), STERITALC® (Talc), STIVARGA® (Regorafenib), Sunitinib Malate, SUTENT® (Sunitinib Malate), SYLATRON® (Peginterferon Alfa-2b), SYLVANT® (Siltuximab), Synribo SYNRIBO® (Omacetaxine Mepesuccinate), TABLOID® (Thioguanine), TAC, TAFINLAR® (Dabrafenib), TAGRISSO® (Osimertinib), Talc, Talimogene Laherparepvec, Tamoxifen Citrate, TARABINE PFS® (Cytarabine), TARCEVA® (Erlotinib Hydrochloride), TARGRETIN® (Bexarotene), TASIGNA® (Nilotinib), TAXOL® (Paclitaxel), TAXOTERE® (Docetaxel), TECENTRIQ® (Atezolizumab), TEMODAR® (Temozolomide), Temozolomide, Temsirolimus, Thalidomide, THALOMID® (Thalidomide), Thioguanine, Thiotepa, Tisagenlecleucel, TOLAK® (Fluorouracil-Topical), Topotecan Hydrochloride, Toremifene, TORISEL® (Temsirolimus), Tositumomab and Iodine I 131 Tositumomab, TOTECT® (Dexrazoxane Hydrochloride), TPF, Trabectedin, Trametinib, Trastuzumab, TREANDA® (Bendamustine Hydrochloride), Trifluridine and Tipiracil Hydrochloride, TRISENOX® (Arsenic Trioxide), TYKERB® (Lapatinib Ditosylate), UNITUXIN® (Dinutuximab), Uridine Triacetate, VAC, Vandetanib, VAMP, VARUBI® (Rolapitant Hydrochloride), VECTIBIX® (Panitumumab), VeIP, VELBAN® (Vinblastine Sulfate), VELCADE® (Bortezomib), VELSAR® (Vinblastine Sulfate), Vemurafenib, VENCLEXTA® (Venetoclax), Venetoclax, VERZENIO® (Abemaciclib), VIADUR® (Leuprolide Acetate), VIDAZA® (Azacitidine), Vinblastine Sulfate, VINCASAR PFS® (Vincristine Sulfate), Vincristine Sulfate, Vincristine Sulfate Liposome, Vinorelbine Tartrate, VIP, Vismodegib, VISTOGARD® (Uridine Triacetate), VORAXAZE® (Glucarpidase), Vorinostat, VOTRIENT® (Pazopanib Hydrochloride), VYXEOS® (Daunorubicin Hydrochloride and Cytarabine Liposome), WELLCOVORIN® (Leucovorin Calcium), XALKORI® (Crizotinib), XELODA® (Capecitabine), XELIRI, XELOX, XGEVA® (Denosumab), XOFIGO® (Radium 223 Dichloride), XTANDI® (Enzalutamide), YERVOY® (Ipilimumab), YONDELIS® (Trabectedin), ZALTRAP® (Ziv-Aflibercept), ZARXIO® (Filgrastim), ZEJULA® (Niraparib Tosylate Monohydrate), ZELBORAF® (Vemurafenib), ZEVALIN® (Ibritumomab Tiuxetan), ZINECARD® (Dexrazoxane Hydrochloride), Ziv-Aflibercept, ZOFRAN® (Ondansetron Hydrochloride), ZOLADEX® (Goserelin Acetate), Zoledronic Acid, ZOLINZA® (Vorinostat), ZOMETA® (Zoledronic Acid), ZYDELIG® (Idelalisib), ZYKADIA® (Ceritinib), and/or ZYTIGA® (Abiraterone Acetate). The treatment methods can include or further include checkpoint inhibitors including, but are not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep).

In some embodiments, the cancer includes, but is not limited to acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

### EXAMPLES

### Example 1: Detection of Exosomal TF-Ag-α via a Compact SPR Biosensor

The expression of exosomal TF-Ag-α was measured using the SPR sensing mechanism. As shown in FIG. 1A, the surface of the gold coated glass slide was modified with a mixture of PEG200 and biotin-PEG1000 (10 mM, 3:1 molar ratio) to provide hydrophilicity and prevent nonspecific binding. Then neutravidin (50 μg/mL) and biotinylated JAA-F11 antibodies (50 μg/mL) were applied sequentially to attach JAA-F11 antibodies on the surface of the biochip via the avidin-biotin interaction.

A compact SPR biosensor was developed to offer highly sensitive, label-free and real-time sensing of exosomal TF-Ag-α. A polarized laser beam was guided by the prism and reached the biochip that sat on top of the prism. The angle of the incident laser was adjusted to trigger SPR on the surface of the gold film, and the intensity of the reflected laser beam was recorded by a photodetector (FIG. 1A). Water and PBS were first applied to collect baseline signals. Then serum samples were added and incubated on the biochip for 1 h to allow the capture of exosomes expressing TF-Ag-α via JAA-F11 antibodies. The binding of exosomes to JAA-F11 antibodies changed the local refractive index, affected SPR and increased the intensity of the reflected laser beam. After washing off unbound exosomes, the signals from exosomes expressing TF-Ag-α were recorded. The expression of exosomal TF-Ag-α was calculated using the following equation.

$$Expression of TEX TF - Ag - α = {( \frac{{I}_{TEX TF - Ag - α} - {I}_{PBS}}{{I}_{PBS} - {I}_{water}} )}_{JAA - F 11 antibody modified biochip} - {( \frac{{I}_{TEX TF - Ag - α} - {I}_{PBS}}{{I}_{PBS} - {I}_{water}} )}_{IgG 3 control antibody modified biochip}$$

Representative SPR curves for the measurements of exosomal TF-Ag-α expression in sera from a stage II lung cancer patient, a stage 0 breast cancer patient and two normal controls were shown in FIG. 1B, FIG. 1C, and FIGS. 8A-F, respectively. The SPR assay was completed in approximately 1 h and used as low as 10 μL serum.

To confirm TF-Ag-α is mainly carried by exosomes, SEM was first used to visualize exosomes expressing TF-Ag-α captured on the biochip surface. Individual exosomes were observed as shown in FIGS. 1D and 1E. Next, the SPR assay was used to measure TF-Ag-α levels in exosome-depleted serum samples (n=10, 5 lung cancer patients and 5 breast cancer patients). Little signal was detected in exosome-depleted serum samples, but strong signals were observed in the same serum samples without exosome depletion (FIG. 1F). Finally, two exosome isolation kits were used, total exosome isolation kit (from serum) and ExoRNeasy Midi kit, to isolate exosomes from serum samples (n=4, 2 lung cancer patients and 2 breast cancer patients). The TF-Ag-α expression of isolated exosomes was measured by SPR assay at the same concentration of 3.29×1011 exosome/mL. Compared with exosome-depleted serum samples, significantly higher levels of TF-Ag-α were observed in exosomes isolated using both kits (FIG. 9). Results from both SEM and SPR assay demonstrated that exosomes do carry TF-Ag-α.

### Example 2: Limit of Detection (LOD) and Reproducibility of SPR Assay

The LODs of the SPR assay were characterized using exosomes derived from A549 NSCLC cells and MDA-MB-231 breast cancer cells. Cell-derived exosomes (40 μL) were spiked in serum from a normal control (10 μL, H9614, Sigma-Aldrich) and applied on the biochip at various concentrations ranging from 0 (blank control) to 10¹¹ exosomes/mL. The blank control was the exosomes isolated from cell culture medium that went through the same procedure but with no cells. The LOD was calculated as the mean of the blank control plus 3 times of the standard deviation. For A549 NSCLC cell-derived exosomes, the SPR assay detected exosomal TF-Ag-α with the LOD of 5×10⁹ exosomes/mL and the linear range from 5×10⁹ to 10¹¹ exosomes/mL (FIG. 10A). For MDA-MB-231 breast cancer cell-derived exosomes, the SPR assay detected exosomal TF-Ag-α with the LOD of 10⁹ exosomes/mL and the linear range from 10⁹ to 10¹¹ exosomes/mL (FIG. 10B). The previous results indicated that the exosome concentration in serum is 10¹² to 10¹³ exosomes/mL. Hence, the SPR assay may detect as low as 0.01% exosomes that express TF-Ag-α in total exosomes, demonstrating its high sensitivity.

To determine the repeatability and reproducibility of SPR assay, the levels of exosomal TF-Ag-α in serum samples from 3 human subjects were measured by two different operators on 3 different days, 3 replicates per day. The three human subjects consisted of one normal control, one lung cancer patient and one breast cancer patient. As shown in FIG. 11A-C, for serum samples from both cancer patients, the inter-day CV and intra-day CV for operator 1 were ≤18.46% and ≤13.55% respectively, and the inter-day CV and intra-day CV for operator 2 were ≤9.27% and ≤5.93% respectively. For the serum from normal control, because the expression of exosomal TF-Ag-α was below the detection limit, as expected, relatively large CV was observed. The results obtained from the two operators showed a very good correlation with Pearson correlation coefficient of 0.92. These results demonstrated that SPR assay had excellent repeatability and reproducibility.

### Example 3: Evaluation of Exosomal TF-Ag-α in Lung Cancer Diagnosis

The diagnostic value of exosomal TF-Ag-α in detecting lung cancer was first evaluated using cell-derived exosomes. Exosomes derived from A549 NSCLC cells, DMS-114 SCLC cells and BEAS-2B human normal bronchial epithelial cells were applied on the biochips at the same concentration of 4×10¹⁰ exosomes/mL. The expression of TF-Ag-α was 2.1-fold and 2.2-fold higher in exosomes from A549 cells and DMS-114 cells respectively than in those from BEAS-2B cells (FIG. 2A).

Next, the diagnostic performance of exosomal TF-Ag-α was evaluated using serum samples from a training set of lung cancer patients and matched controls. The training set consisted of 41 normal controls (including 20 patients at low risk of lung cancer and 21 patients at high risk of lung cancer) and 40 lung cancer patients. Two major subtypes of lung cancer, i.e., NSCLC (n=30) and SCLC (n=10) were included. Among them, 18 patients had early stage (I/II) lung cancer and 22 patients had late stage (III/IV) lung cancer. The patient characteristics were provided in TABLE 1. The serum sample (10 μL) was diluted with 40 μL PBS and applied on the biochip for the detection of exosomal TF-Ag-α. A significantly higher levels of exosomal TF-Ag-α was observed in all lung cancer groups than normal controls (FIG. 2B and FIG. 2C). Among all normal controls, no significant difference was observed between the low-risk group and the high-risk group. A higher exosomal TF-Ag-α levels in sera was observed from late stage (III/IV) lung cancer patients than early stage (I/II) lung cancer patients (FIG. 2B). And there was no significant difference in the expression of exosomal TF-Ag-α between cancer subtypes (FIG. 2C). The lack of significant difference between cancer stages or subtypes may be due to the small sample size, which requires further investigation and validation in large patient cohorts. The other possible reason may be because TF-Ag-α is involved in homotypic aggregation and metastasis. Homotypic aggregation may decrease exosome release of TF-Ag-α as tumor associated TF-Ag-α is a critical molecule in initiating tumor/endothelial cell adhesion and facilitating cancer metastasis.

TABLE 1
Characteristics of lung cancer patients (training set).

| Characteristics | | N (%) |
|---|---|---|
| Total patients | | 81 |
| Normal controls (low-risk) | | 20(24.7) |
| Normal controls (high-risk) | | 21(25.9) |
| Cancer | | 40(49.4) |
| | Normal controls | |
| | (both low-risk and | Lung cancer |
| | high-risk patients) | patients |
| Gender | | |
| Males | 24(29.6) | 20(24.7) |
| Females | 17(20.9) | 20(24.7) |
| Age (in years) | | |
| Mean | 59.9 | 64.3 |
| Median | 58 | 63 |
| Range | 42-77 | 47-78 |
| Stage | | |
| I | | 10(25.0) |
| II | | 8(20.0) |
| III | | 14(35.0) |
| IV | | 8(20.0) |
| Morphology | | |
| NSCLC | | 30(75.0) |
| SCLC | | 10(25.0) |

Receiver operating characteristic (ROC) analysis was used to determine the sensitivity, specificity, area under curve (AUC) of exosomal TF-Ag-α in lung cancer diagnosis (FIGS. 3A-3F). Exosomal TF-Ag-α distinguished lung cancer patients (n=40) from normal controls (n=41) with sensitivity of 0.98, specificity of 0.85 and AUC of 0.95 at cutoff value of 0.441 (FIG. 3A). To further refine the analysis lung cancer patients were divided into early stage (stage I/II) and late stage (stage III/IV) groups. Exosomal TF-Ag-α distinguished early stage lung cancer patients (n=18) from normal controls (n=41) with sensitivity of 1.00, specificity of 0.85 and AUC of 0.94 (FIG. 3B), and late stage lung cancer patients (n=22) from normal controls (n=41) with sensitivity of 0.91, specificity of 0.95 and AUC of 0.95 (FIG. 3C). Lung cancer patients were also divided based on cancer subtype, i.e., NSCLC and SCLC. Exosomal TF-Ag-α differentiated NSCLC (n=30) from normal controls (n=41) with sensitivity of 0.97, specificity of 0.85 and AUC of 0.95 (FIG. 3D), and SCLC (n=10) from normal controls (n=41) with sensitivity of 1.00, specificity of 0.85 and AUC of 0.94 (FIG. 3E).

To confirm the diagnostic performance of exosomal TF-Ag-α, a blinded study was performed using a test cohort of lung cancer patients and controls. The test set consisted of 6 normal controls and 20 lung cancer patients. Two major subtypes of lung cancer, i.e., NSCLC (n=14) and SCLC (n=6) were included. Among them, 10 patients had early stage (I/II) lung cancer and 10 patients had late stage (III/IV) lung cancer. The patient characteristics were provided in TABLE 2. The levels of exosomal TF-Ag-α expression were measured by both operator 1 and operator 2. As shown in FIGS. 4A-4C, exosomal TF-Ag-α expression was significantly higher in serum samples from lung cancer patients at all stages than normal controls. Based on the observed exosomal TF-Ag-α levels, an ROC curve was plotted, and AUC was determined to be 1.00, Compared with the training set (AUC=0.95), exosomal TF-Ag-α showed a higher diagnostic accuracy in the test set (AUC=1.00). When the cutoff value (0.441) identified from the training set was applied, the sensitivity and specificity of exosomal TF-Ag-α in distinguishing lung cancer patients from normal controls were 0.90 and 1.00, respectively. These results collectively demonstrated that exosomal TF-Ag-α was a highly sensitive and specific biomarker for the diagnosis of lung cancer.

TABLE 2
Characteristics of lung cancer patients (test set).

| Characteristics | | N (%) |
|---|---|---|
| Total patients | | 26 |
| Normal controls (low-risk) | | 6(23.1) |
| Cancer | | 20(76.9) |
| | Normal | Lung cancer |
| | controls | patients |
| Gender | | |
| Males | 3(11.5) | 10(38.5) |
| Females | 3(11.5) | 10(38.5) |
| Age (in years) | | |
| Mean | 62.7 | 59.7 |
| Median | 62.5 | 62 |
| Range | 48-77 | 41-70 |
| Stage | | |
| I | | 5(25.0) |
| II | | 5(25.0) |
| III | | 5(25.0) |
| IV | | 5(25.0) |
| Morphology | | |
| NSCLC | | 14(70.0) |
| SCLC | | 6(30.0) |

### Example 4: Evaluation of Exosomal TF-Ag-α in Breast Cancer Diagnosis

The diagnostic value of exosomal TF-Ag-α in detecting breast cancer was evaluated using both cell-derived exosomes and human serum samples. Exosomes derived from MDA-MB-231, MCF-7 breast cancer cells and MCF-10A normal cells were applied on the biochip at the concentration of 10¹¹ exosomes/mL. Compared with exosomes from MCF-10A cells, the expression of TF-Ag-α was 13.5-fold higher in exosomes from MDA-MB-231 cells and 7.5-fold higher in exosomes from MCF-7 cells (FIG. 5A).

Next, the levels of exosomal TF-Ag-α in serum samples from a training set of patients were measured using the SPR assay. The training set included 14 normal controls, 16 patients with benign breast conditions and 70 breast cancer patients. Benign breast conditions were atypical ductal hyperplasia, atypical lobular hyperplasia, fibroadenoma, fibrocystic change and intraductal papilloma. All 5 stages of breast cancer were included (stage 0: n=12; stage I: n=14; stage II: n=15, stage III: n=14; stage IV: n=15). All major subtypes of breast cancer, i.e., luminal A/B (n=44), triple negative (n=9) and HER2-positive (n=17) were included. The patient characteristics were provided in TABLE 3. Similar to lung cancer, a significantly higher levels of exosomal TF-Ag-α was observed in all breast cancer groups than normal controls and patients with benign breast conditions. No significant difference was observed between normal controls and patients with benign breast conditions, and between cancer stages or subtypes (FIGS. 5B and 5C).

TABLE 3
Characteristics of breast cancer patients (training set).

| Characteristics | | n (%) |
|---|---|---|
| Total patients | | 99 |
| Normal controls (no breast conditions) | | 14(14.1) |
| Normal controls (benign breast conditions) | | 15(15.2) |
| Cancer Patients | | 70(70.7) |
| | Normal controls | Breast |
| | (Including patients with | cancer |
| | benign breast conditions) | patients |
| Gender | | |
| Males | 0 | 0 |
| Females | 29(29.3) | 70(70.7) |
| Age (in years) | | |
| Mean | 55.9 | 57.1 |
| Median | 55 | 57 |
| Range | 36-77 | 30-80 |
| Stage | | |
| 0 | | 12(12.1) |
| I | | 14(14.1) |
| II | | 15(15.2) |
| III | | 14(14.1) |
| IV | | 15(15.2) |
| Morphology | | |
| Luminal A/B | | 44(44.4) |
| Triple negative | | 9(9.1) |
| HER2 enriched | | 17(17.2) |

ROC analysis was performed to determine the sensitivity, specificity and AUC of exosomal TF-Ag-α in breast cancer diagnosis (FIGS. 6A-6G). Exosomal TF-Ag-α distinguished breast cancer patients (n=70) from normal controls (n=29) with sensitivity of 0.90, specificity of 0.93 and AUC of 0.97 at cutoff value of 0.484 (FIG. 6A). Breast cancer patients were divided into non-metastatic group (stage 0-III) and metastatic group (stage IV). Exosomal TF-Ag-α distinguished non-metastatic breast cancer patients (n=55) from normal controls (n=29) with sensitivity of 0.93, specificity of 0.97 and AUC of 0.98 (FIG. 6B), and metastatic breast cancer patients (n=15) from normal controls (n=29) with sensitivity of 0.93, specificity of 0.86 and AUC of 0.93 (FIG. 6C). For various subtypes of breast cancer, exosomal TF-Ag-α differentiated luminal A/B (n=44) from normal controls (n=29) with sensitivity of 0.86, specificity of 0.97 and AUC of 0.96 (FIG. 6D), triple negative (n=9) from normal controls (n=29) with sensitivity of 0.89, specificity of 1.00 and AUC of 0.99 (FIG. 6E), and HER2-positive (n=17) from normal controls (n=29) with sensitivity of 0.94, specificity of 0.97 and AUC of 0.99 (FIG. 6F). To confirm the diagnostic performance of exosomal TF-Ag-α, a blinded study was performed using a test cohort of breast cancer patients and controls. The test set consisted of 5 normal controls (including 2 patients with benign breast conditions) and 25 breast cancer patients. All 5 stages of breast cancer (stage 0-IV, n=5 per stage) and all major subtypes of breast cancer (19 luminal A/B, 3 triple negative and 3 HER2-positive) were included. The patient characteristics were provided in TABLE 4. The levels of exosomal TF-Ag-α expression were measured by both operator 1 and operator 2. As shown in FIGS. 7A-7C, exosomal TF-Ag-α expression was significantly higher in serum samples from breast cancer patients than normal controls. Based on the observed exosomal TF-Ag-α levels, an ROC curve was plotted, and AUC was determined to be 1.00. Compared with the training set (AUC=0.97), exosomal TF-Ag-α showed a higher diagnostic accuracy in the test set (AUC=1.00). When the cutoff value (0.484) identified from the training set was applied, the sensitivity and specificity of exosomal TF-Ag-α in distinguishing breast cancer patients from normal controls were 0.92 and 1.00, respectively. All results demonstrated that exosomal TF-Ag-α was a sensitive biomarker for breast cancer diagnosis.

TABLE 4
Characteristics of breast cancer patients (test set).

| Characteristics | | n (%) |
|---|---|---|
| Total patients | | 30 |
| Normal controls (no breast conditions) | | 3(10.0) |
| Normal controls (benign breast conditions) | | 2(6.67) |
| Cancer Patients | | 25(83.3) |
| | Normal controls | Breast |
| | (Including patients with | cancer |
| | benign breast conditions) | patients |
| Gender | | |
| Males | 0(0) | 0(0) |
| Females | 5(16.7) | 25(83.3) |
| Age (in years) | | |
| Mean | 56.8 | 60.8 |
| Median | 50 | 62 |
| Range | 47-77 | 35-78 |
| Stage | | |
| 0 | | 5(20.0) |
| I | | 5(20.0) |
| II | | 5(20.0) |
| III | | 5(20.0) |
| IV | | 5(20.0) |
| Morphology | | |
| Luminal A/B | | 19(76.0) |
| Triple negative | | 3(12.0) |
| HER2 enriched | | 3(12.0) |

### Example 5: Evaluating the Use of TF-Ag-α Expressing Tumor-Derived Exosomal PD-L1 to Predict and Monitor Response to Cancer Immunotherapy

A SPR-based assay is developed to capture TEX and quantify TEX PD-L1 for ICI treatment response prediction. A unique monoclonal antibody is utilized, JAA-F11, with high specificity to TF-Ag-α to capture TEX expressing TF-Ag-α (TEX^{TF-Ag-α}). After the capture of TEX^{TF-Ag-α} the SPR assay was used to measure the expression of TF-Ag-α. Then APC fluorescent dye labelled anti-PD-L1 antibody was added to quantify the expression of TEX^{TF-Ag-α} PD-L1.

To perform the SPR-based assay, a SPR biochip was first prepared by coating the surface of a glass slide with 2 nm Ti and 49 nm Au films. Then the surface was modified with a mixture of polyethylene glycol (PEG200) and biotin-PEG1000. After washing off unbound PEG, the surface was treated with avidin. After washing off unbound avidin, biotinylated JAA-F11 antibodies were tethered on the surface through biotin-avidin interaction (FIG. 12A). The JAA-F11 antibodies uniquely bind to the alpha-linked TF-Ag (i.e. TF-Ag-α) but not the beta-linked present in glycolipids in normal tissues. JAA-F11 antibody is currently under the protection of a patent titled “Therapeutic Use of anti-TF-antigen Antibody”. This patent has been issued in (U.S. Pat. No. 7,374,755, issued on May 20, 2008), Europe (patent #: 1789027), Japan (patent #: 4904266) and Canada (patent #: 2582252).

A home built SPR instrument was used to measure the levels of TEX^{TF-Ag-α} PD-L1 (FIGS. 12B and 12C). Water and PBS were first applied on the biochip to collect background signals (I_{water} and I_{PBS}). Then 10 uL serum was added to 40 uL PBS and the mixture was applied and incubated on the biochip for 1 h. TEX^{TF-Ag-α} is captured by JAA-F11 antibodies on the biochip surface. The binding of TEX^{TF-Ag-α} to JAA-F11 antibodies change the local refractive index, affect the optical properties of the surface plasmon modes, and thus permit the optical detection of TEX^{TF-Ag-α} After washing off unbound exosomes with PBS, the intensity of reflected light is recorded as I_{TEX TF-Ag-α}. The expression of TF-Ag-α was calculated using the following equation:

$$Expression of TEX TF - Ag - α = \frac{{I}_{TEX TF - Ag - α} - {I}_{PBS}}{{I}_{PBS} - {I}_{water}}$$

Next, APC fluorescent dye labelled anti-PD-L1 antibody (APC-PD-L1) was added and incubated on the biochip for 1 h. After washing off excess APC-PD-L1 antibody, the fluorescence intensity of APC (I_{after APC-PD-L1}) was recorded and used to calculate the expression of TEX^{TF-Ag-α} PD-L1 using the equation:

$$Expression of {TEX}^{TF - Ag - α} PD - L 1 = ( \frac{{I}_{after APC - PD - L 1} - {I}_{before APC - PD - L 1}}{{I}_{before APC - PD - L 1}} ) / ( Expression of TEX TF - Ag - α ) = ( \frac{{I}_{after APC - PD - L 1} - {I}_{before APC - PD - L 1}}{{I}_{before APC - PD - L 1}} ) / ( \frac{{I}_{TEX TF - Ag - α} - {I}_{PBS}}{{I}_{PBS} - {I}_{water}} )$$

in which I_{before APC-PD-L1} was the fluorescence signal of background before APC-PD-L1 antibody was added.

It is demonstrated that the potential utility of TEX^{TF-Ag-α} PD-L1 in predicting response to anti-PD-1/PD-L1 therapy (e.g. pembrolizumab, nivolumab, etc) in advanced NSCLC patients. The levels of TEX^{TF-Ag-α} PD-L1 were measured in the serum samples from 17 responders (complete response and partial response) and 36 non-responders (stable disease and progression) collected at pre-treatment baseline and at 3 weeks post treatment, i.e. at the end of first treatment cycle. To demonstrate that the selective capture of TEX indeed increases predictive accuracy, the predictive performance was compared between TEX^{TF-Ag-α} PD-L1 and total exosomal PD-L1 (FIGS. 13A-13E). Significantly lower TEX^{TF-Ag-α} PD-L1 expression was observed in responders (n=17) than non-responders (n=36) at pre-treatment baseline (FIG. 13A). The levels of TEX^{TF-Ag-α} PD-L1 were increased in responders and were slightly decreased in non-responders. The fold change of TEX^{TF-Ag-α} PD-L1 expression was significantly higher in responders (4.05-fold) than non-responders (0.8-fold) at 3 weeks post treatment (FIG. 13C). Similar results were also observed with total exosomal PD-L1 (FIGS. 13B and 13D). However, when ROC analysis was performed to determine the sensitivity, specificity and area under curve (AUC) of TEX^{TF-Ag-α} PD-L1 and total exosomal PD-L1, TEX^{TF-Ag-α} PD-L1 better distinguished responders from non-responders at both pre-treatment baseline and at 3 weeks post treatment (FIG. 13E). These results strongly suggest that TEX^{TF-Ag-α} PD-L1 is a more effective biomarker than total exosomal PD-L1 in predicting response to anti-PD-1/PD-L1 therapy. These results strongly indicated that to achieve high predictive performance, it is important to selectively capture TEX and remove interference from other exosomes.

To further demonstrate the innovation of using TF-Ag-α to select TEX and thus greatly enhancing the predictive performance of PD-L1, LG3BP (galectin-3-binding protein) was used to select TEX and evaluated if LG3BP selection improved the predictive performance of PD-L1 (FIG. 14A). LG3BP facilitates lung tumor growth and metastasis. LG3BP is a promising biomarker that distinguished benign from malignant lung nodules according to results of the PANOPTIC (Pulmonary Nodule Plasma Proteomic Classifier) Trial.

The levels of TEX^{LG3BP} PD-L1 were measured in the serum samples from a total of 29 NSCLC patients who received ICI (i.e., pembrolizumab) therapy. Among them, 15 were responders (complete response and partial response) and 14 were non-responders (stable disease and progression). Serum samples were collected at pre-treatment baseline and at 3 weeks post treatment. As shown in FIGS. 14B and 14C, no significant difference was observed between responders and non-responders in the expression of TEX^{LG3BP} PD-L1 and TEX^{LG3BP}PD-L1 fold change. These results demonstrated that LG3BP is inferior to TF-Ag-α in selecting TEX and thus it cannot assist in improving the predictive performance of PD-L1.

### Discussion

Lab tests, medical imaging and biopsies are routinely used in clinical cancer diagnosis. For example, low-dose chest CT (LDCT) is a common lung cancer screening modality. Mammography and ultrasound serve as the main methods for breast cancer screening. However, high false positive rate, risk of overdiagnosis, and unnecessary radiation exposure are the main challenges. Although computer-assisted methods such as deep learning have been developed to assist LDCT and mammograms, and great progress has been made so far, accuracy of these approaches is still poor and subjective. In fact, tissue biopsy is the gold standard in clinic, however, it is an invasive procedure, cannot be easily repeated in certain patients such as those with lung cancer, and is challenged by tumor heterogeneity. Liquid biopsy detects circulating cancer biomarkers in body fluids, such as blood, urine and saliva. It is a minimal- or non-invasive test, provides supplementary information in the wake of medical imaging or tissue biopsy, and assists with cancer screening, diagnosis, and treatment response monitoring. However, current circulating blood-based biomarkers, such as CEA, PSA, CA125 and CAT9-1 suffer from poor sensitivity and specificity. Recent studies have demonstrated that circulating tumor cells (CTCs), circulating tumor DNAs (ctDNAs) and TEXs are promising biomarkers to enable highly sensitive and specific cancer diagnosis. Compared with CTCs and ctDNAs, TEXs are particularly suitable for cancer diagnosis because TEXs exist in much larger quantities than CTCs and ctDNAs, and thus may provide higher diagnostic sensitivity. TEXs are much more stable than CTCs and ctDNAs and do not require immediate nor special sample processing, which are necessary attributes for cost-effective, large-scale, routine local or central lab-based cancer tests. This stability allows for biomarker discovery/validation using samples in biorepositories. TEXs are secreted by living cancer cells, unlike ctDNA from ‘dying cells’, thus providing a more precise portrait of tumor status.

The discovery of TEX markers represents a new technological approach in cancer liquid biopsy platforms. Many TEX cargos, such as microRNAs and proteins have been extensively investigated. However, TEX carbohydrate markers have not yet been explored. It is shown that TF-Ag-α expression in tissue was detected by JAA-F11 antibody in 85% of 1269 samples of breast, lung, prostate, colon, bladder, and ovarian cancer, but not on normal tissues. Specifically, for lung and breast cancers, 84% of 235 lung cancer samples and 88% of 444 breast cancer samples tested positive for TF-Ag-α expression. These results suggest that TF-Ag-α may be a pan-cancer biomarker with high sensitivity and specificity. Exosomes carry TF-Ag-α and exosomal TF-Ag-α could be used as a liquid biopsy biomarker for cancer diagnosis is discovered. In the current invention, lung and breast cancer are selected as the disease models and it has been demonstrated that exosomes carry TF-Ag-α. In order to translate the discovery into clinical settings, a highly sensitive SPR assay is developed to detect exosomal TF-Ag-α directly in serum without sample preparation. Results from the LOD experiments showed that the SPR assay may detect as low as 0.01% exosomes that express TF-Ag-α in total exosomes. The SPR assay also showed excellent intra-day, inter-day and inter-operator reproducibility. The SPR assay embraced a user-friendly design, only requiring basic understanding of SPR sensing mechanism and experience in using a micropipette to perform this assay. With a total of 233 patients and controls in both training and test sets, exosomal TF-Ag-α distinguished lung cancer patients (stage I-IV) and breast cancer patients (stage 0-IV) from normal controls with AUC≥0.95 and ≥0.97, respectively. These results collectively demonstrated the great potential of exosomal TF-Ag-α in lung and breast cancer liquid biopsy. Due to its expression across various malignancies however, additional platforms to establish the tissue type of origin are necessary to confirm the specific cancer diagnosis.

Expanding the cancer type to include more cancers such as colon cancer, prostate cancer and ovarian cancer can be done because of the high expression of TF-Ag-α in the cancer tissues captured in this invention. In addition, exosomal TF-Ag-α measurement can be utilized as a non-invasive companion diagnostic platform for cancer-specific assessment of other membrane biomarkers expressed on the cell surface, e.g., to evaluate dynamic changes in HER2 expression status. Lastly, a humanized version of JAA-F11 (hJAA-F11) with high specificity to TF-Ag-α and low immunogenicity has been developed for clinical administration for imaging and therapy. The exosomal TF-Ag-α assay developed in this work may also be used as a companion test to select patients for hJAA-F11 therapy and monitor treatment response.

It has been demonstrated that exosomes carry TF-Ag-α, and exosomal TF-Ag-α is a cancer specific marker. TF-Ag-α can be used to select TEX and thus significantly improve the accuracy in predicting response to cancer therapy, such as ICIs. The far-reaching impact of using TF-Ag-α to select TEX and then measuring TEX biomarkers will go beyond the prediction of response to ICI therapy. Since TEX TF-Ag-α may reflect tumor burden, it can be used to monitor response to all cancer therapies such as chemotherapy, radiation therapy, targeted therapy and surgery. Moreover, given the ubiquitous expression of TF-Ag-α in malignancies, it can be used to distinguish cancer-specific exosomes in the development of complementary exosome-based assays as a surrogate to tissue-based analysis of target antigen expression by immunohistochemistry. In addition, a humanized version of JAA-F11 (hJAA-F11) with high specificity to TF-Ag-α and low immunogenicity has been developed for clinical administration. TEX TF-Ag-α may also be used as a companion test to select patients for hJAA-F11 therapy and monitor treatment response. TEX TF-Ag-α may also be used to determine minimal residual disease. For decades, our risk prediction models are chiefly driven by the TNM staging criteria and histopathologic parameters. A sensitive and specific non-invasive test for determining minimal residual disease in these settings can optimize our selection of patients who will benefit the most from costly and potentially toxic adjuvant systemic therapies that currently benefit only a small proportion of patients.

A major challenge in developing exosomes based cancer liquid biopsy is to capture TEX and differentiate them from exosomes released by normal cells. Without this feature in separating cancer-specific exosomes, current exosomes-based assays may result in the tests having poor sensitivity and specificity. The enrichment process of TEXs can be cumbersome and requires more sensitive techniques to distinguish true signals (TEX) vs noise arising from non-exosomes and exosomes derived from normal cells. Utilizing JAA-F11 to selectively identify TEX, i.e., exosomes containing TF-Ag-α can differentiate true signals versus those emanating from contaminants. This enrichment process for the capture and identification of TEX per se provides a methodologic improvement on existing methods for capture of TEX.

### Materials and Methods

Chemicals: Methyl-(PEG)4-SH (PEG200, MW 224 Da, 26132) was purchased from Sigma-Aldrich (St. Louis, MO). Biotin-PEG1000-SH (Biotin-PEG1000, MW 1000 Da, PG2-BNTH-1k) was purchased from Nanocs (Boston, MA). NeutrAvidin protein (31000), EZ-Link™ Micro Sulfo-NHS-LC-Biotinylation Kit (21935), biotin labelled anti-PD-L1 antibody (13-5983-82), APC fluorescent dye labelled anti-PD-L1 antibody (APC-PD-L1, 17-5983-42) and total exosome isolation (from cell culture medium) kit (4478359) were purchased from Thermo Fisher Scientific (Rockford, IL). JAA-F11 mouse monoclonal antibodies (anti-TF-Ag-α mAb) were supplied by For-Robin Inc. (Williamsville, NY). Mouse IgG3 antibodies (0105-01, RRID: AB_2793898) were purchased from SouthemBiotech (Birmingham, AL). ASYLGARD 184 silicone elastomer kit (PDMS, 2065622) was purchased from Dow Corning (Midland, MI).

Cell Culture: A549 human NSCLC cells (RRID: CVCL_0023) and BEAS-2B human normal bronchial epithelial cells (RRID: CVCL_0168) were cultured in RPMI 1640 medium (11875093, Thermo Fisher Scientific) supplemented with 10% v/v fetal bovine serum (FBS, 26140079, Thermo Fisher Scientific) and 1% v/v penicillin-streptomycin (PS, 15140122, Thermo Fisher Scientific). A549 and BEAS-2B cells were seeded at 1.0×106 and 1.5×106 cells/P100 dish, respectively, and subcultured every 2-3 days.

DMS-114 human small cell lung cancer (SCLC) cells (RRID: CVCL_1174) were cultured in Waymouth's medium (11220035, Thermo Fisher Scientific) supplemented with 15% v/v exosome-depleted FBS and 1% v/v PS. The exosome-depleted FBS was prepared by centrifuging FBS at 100,000 g for 19 h at 4 °C. DMS-114 cells were seeded at 1.5×106 cells/P100 dish and subcultured every 3-4 days.

MCF-10A human non-tumorigenic mammary epithelial cells (RRID: CVCL_0598) were cultured in DMEM/F12 (11330-032, Thermo Fisher Scientific) supplemented with 5% v/v horse serum (16050-122, Thermo Fisher Scientific), 20 ng/mL EGF (AF-100-15, Peprotech), 0.5 mg/mL hydrocortisone (H-0888, Sigma-Aldrich), 100 ng/mL cholera toxin (C-8052, Sigma-Aldrich), 10 μg/mL insulin (I-1882, Sigma-Aldrich) and 1% v/v PS. MCF-7 (RRID: CVCL_0031) and MDA-MB-231 (RRID:CVCL_0062) human breast cancer cells were cultured in DMEM (11995-065, Thermo Fisher Scientific) with 10% v/v FBS and 1% v/v PS. MCF-10A cells were seeded at 2×106 cells/P100 dish. MDA-MB-231 and MCF-7 cells were seeded at 1.5×10⁶ cells/P100 dish. All cells were subcultured every 2-3 days.

All cells used in this study were obtained from American Type Culture Collection (ATCC, Manassas, Virginia). Short tandem repeat profiling was used for cell line authentication. MycoFluor™ Mycoplasma Detection Kit (M7006, Thermo Fisher Scientific) was used to confirm all cell cultures were free of mycoplasma. All cells were used within 2 months after thawing.

Isolation of Exosomes from Cell Culture-Conditioned Media: A549, BEAS-2B, MCF-10A, MCF-7 and MDA-MB-231 cells were seeded and allowed to grow to 80% confluence in P100 dishes. Then the cells were washed with PBS and cultured in media containing no FBS or horse serum for 2 days. The cell culture-conditioned media were collected and centrifuged at 2,500 g for 10 min, 4,000 g for 30 min, and 10,000 g for 1 h at 4 °C. The total exosome isolation kit was added to the cell culture-conditioned media at 1:2 volume ratio according to the manufacturer's protocol. After incubation at 4 °C overnight, the mixture was centrifuged at 10,000 g for 1 h at 4 °C to pellet the exosomes. Exosome pellets were resuspended in PBS for future applications. DMS-114 cells were seeded and allowed to grow to 80% confluence in P100 dishes in cell culture medium containing exosome-depleted FBS. The cell culture-conditioned media were collected and processed as described above to isolate exosomes for further analyses.

Characterization of Exosomes by Nanoparticle Tracking Analysis (NTA): The NTA system (NanoSight, LM10, Malvern Instruments, Worcestershire, UK) was used to measure cell-derived exosomes' size, size distribution and number concentration. To maintain the accuracy and consistency of the measurements, exosomes were first diluted with PBS until 50-100 nanoparticles can be tracked in the field of view. The setting of measurement parameters was also identical for all measurements. The camera level was set at 14 during the view-capturing process. The detection threshold was set at 6, and the screen gain was set at 8 during the video processing process.

Human Serum Samples: De-identified serum samples and clinical data of normal controls, treatment naïve lung cancer patients, and treatment naïve breast cancer patients were obtained from the Data Bank and Bio Repository Shared Resource (DBBR) at Roswell Park Comprehensive Cancer Center (Buffalo, NY). After obtaining written informed consent from the patients, the DBBR collected high-quality serum samples and associated epidemiological and clinical data. After the serum samples were transferred to the University at Buffalo on dry ice, they were stored at −80 °C. All serum samples were centrifuged at 10,000 g at 4 °C for 30 min to remove debris, and then diluted with PBS at volume ratio of 1:4 before use. Approvals from Institutional Review Boards (IRBs) of Roswell Park Comprehensive Cancer Center and the University at Buffalo were obtained for the use of human serum samples in this study.

Exosome Isolation and Preparation of Exosomes-Depleted Human Serum Samples: Total exosome isolation kit (from serum) (4478360, Thermo Fisher Scientific) was used to isolate exosomes from human serum samples and prepare exosomes-depleted serum samples. Briefly, serum was mixed with the total exosome isolation kit (for serum) at 2:1 volume ratio according to the manufacturer's protocol. After incubation at 4 °C for 30 min, the mixture was centrifuged at 10,000 g for 10 min at room temperature (RT) to pellet exosomes. The supernatant, i.e., exosomes-depleted serum was collected. Exosome pellets were resuspended in 1×PBS.

ExoRNeasy Midi Kit (77144, Qiagen) was used to isolate exosomes from human serum samples. Briefly, 100 uL serum was mixed with 100 uL buffer XBP. The mixture was added into exoEasy spin column and centrifuged at 500 g for 1 min and at 3,000 g for another minute to ensure no liquid was left in the column. Buffer XWP (3.5 mL) was added into the column, and the column was washed by centrifugation at 3,000 g for 1 min. Finally, 100 uL buffer XE (76214, Qiagen) was added to the membrane inside the column and incubated for 1 min. The column was centrifuged at 3,000 g for 5 min to elute exosomes. The elute was reapplied on the membrane, and the column was centrifuged at 3,000 g for another 5 min to maximize the recovery of exosomes.

Fabrication of SPR Biochip: A glass slide (12-550-A3, Fisher Scientific) was sequentially cleaned with acetone, 200 proof ethanol, and DI water using a 10 min sonication for each step. A 2 nm Ti layer and a 49 nm Au layer were then deposited on the glass slide surface by electron-beam evaporation (Kurt J. Lesker Company, USA). The deposition rate for Ti was controlled at 0.8 Å/s and for Au was controlled at 1 Å/s. Finally, the Au-coated glass slide and a piece of PDMS sheet with a 6 mm in diameter hole were treated with oxygen plasma and bound together to complete the assembly of the biochip. The 6 mm in diameter hole served as the sample well.

Surface Modification of SPR Biochip: The PEG mixture (10 mM) was prepared by mixing biotin-PEG1000 with PEG200 at a molar ratio of 1:3 in PBS. The Au-coated surface of the biochip was incubated with 100 μL PEG mixture for 1 h at RT. Excess PEG was washed off with PBS. Then, 100 μL 50 μg/mL NeutrAvidin was added and incubated for 1 h at RT. After unbound NeutrAvidin was washed off with PBS, 100 μL biotinylated mouse JAA-F11 or mouse IgG3 antibodies or anti-PD-L1 antibodies (50 μg/mL in PBS) were added and incubated at 4 °C overnight to conjugate antibodies onto the surface of the biochip through the biotin-avidin interaction. After excess antibodies were removed carefully by PBS washing, the biochip was stored at 4 °C for future use.

Detection of Exosomal TF-Ag-α by the SPR biosensor: The biochip modified with biotinylated mouse JAA-F11 or mouse IgG3 antibodies was placed on the prism of the SPR biosensor (FIG. 12C). A 647 nm laser (30 mW power density) was shed through the sample well at the SPR angle, and the intensity of reflected laser beam (I) was recorded by a photodetector. To measure the expression of exosomal TF-Ag-α, water and PBS were first applied on the biochip. The SPR signals of water (Iwater) and PBS (IPBS) were recorded for 2 min each to establish the baseline measurements.

Then 50 μL cell-derived exosomes or human serum samples (10 μL serum was diluted with 40 μL PBS) were applied on the biochip and incubated at RT for 1 h to allow the capture of exosomes expressing TF-Ag-α by JAA-F11 antibodies. After washing off unbound exosomes, the SPR signals from exosomes expressing TF-Ag-α (I_{exosomal TF-Ag-α}) were recorded for 2 min. The biochip modified with the IgG3 control antibodies was used to measure the same sample, and the signal was used to remove background noise caused by nonspecific binding. The expression of exosomal TF-Ag-α was calculated using the following equation:

$$Expression of TEX TF - Ag - α = {( \frac{{I}_{TEX TF - Ag - α} - {I}_{PBS}}{{I}_{PBS} - {I}_{water}} )}_{JAA - F 11 antibody modified biochip} - {( \frac{{I}_{TEX TF - Ag - α} - {I}_{PBS}}{{I}_{PBS} - {I}_{water}} )}_{IgG 3 control antibody modified biochip}$$

The difference between I_{PBS} and I_{water} was used as the normalization factor to minimize the chip-to-chip variation. To detect total exosomal PD-L1 in serum samples, a biochip modified with biotinylated anti-PD-L1 antibody was used, and the same procedure was used.

Detection of TEX^{TF-Ag-α} PD-L1 by the SPR biosensor: After the detection of exosomal TF-Ag-α by the SPR biosensor as described above, 100 μL APC fluorescent dye labelled anti-PD-L1 antibody (APC-PD-L1, 10 μg/mL in PBS) was added and incubated on the biochip for 1 h. After washing off excess APC-PD-L1 antibody using PBS, the fluorescent signal of APC (I_{after APC-PD-L1}) was recorded by the avalanche photodiode (APD) detector (FIG. 12C). The expression of TEX^{TF-Ag-α} PD-L1 was calculated using the equation:

$$Expression of {TEX}^{TF - Ag - α} PD - L 1 = ( \frac{{I}_{after APC - PD - L 1} - {I}_{before APC - PD - L 1}}{{I}_{before APC - PD - L 1}} ) / ( Expression of TEX TF - Ag - α ) = ( \frac{{I}_{after APC - PD - L 1} - {I}_{before APC - PD - L 1}}{{I}_{before APC - PD - L 1}} ) / ( \frac{{I}_{TEX TF - Ag - α} - {I}_{PBS}}{{I}_{PBS} - {I}_{water}} )$$

wherein I_{before APC-PD-L1} is fluorescence intensity of APC before incubating biochip with anti-APC-PD-L1 antibody.

Detection of total exosomal PD-L1 by the SPR biosensor: The biochip modified with biotinylated anti-PD-L1 antibodies was placed on the prism of the SPR biosensor (FIG. 12C). A 647 nm laser (30 mW power density) was shed through the sample well at the SPR angle, and the intensity of reflected laser beam (I) was recorded by a photodetector. To measure the expression of exosomal PD-L1, water and PBS were first applied on the biochip. The SPR signals of water (I_{water}) and PBS (I_{PBS}) were recorded for 2 min each to establish the baseline measurements.

Then 50 μL human serum samples (10 μL serum was diluted with 40 μL PBS) were applied on the biochip and incubated at RT for 1 h to allow the capture of exosomes expressing PD-L1 by anti-PD-L1 antibodies. After washing off unbound exosomes, the SPR signals from exosomes expressing PD-L1 (I_{exosomal PD-L1}) were recorded for 2 min. The expression of exosomal PD-L1 was calculated using the following equation:

$$Expression of TEX PD - L 1 = ( \frac{{I}_{TEX PD - L 1} - {I}_{PBS}}{{I}_{PBS} - {I}_{water}} )$$

Scanning Electron Microscopy (SEM): After the capture of exosomes expressing TF-Ag-α on the biochip by JAA-F11 antibodies, the unbound exosomes were removed by washing the biochip with PBS 3 times. Then the biochip was dried at 4 °C and coated with a layer of carbon before SEM imaging. Exosomes expressing TF-Ag-α were characterized using a field emission scanning electron microscope (SU-70, Hitachi High Technologies, Schaumburg, IL) with an energy-dispersive X-ray spectrometer (Oxford Instruments, UK).

Statistical Data Analysis: Receiver operating characteristics (ROC) analysis and standard two-tailed t-tests were performed using the SPSS® software platform (Ver. 26, IBM, Armonk, NY).

Cutoff value determination: Using the training cohort, an exact logistic regression model was used to generate an ROC curve and corresponding cross-validated AUC (area under curve) assessing the ability of exosomal TF-Ag-α to discriminate between normal controls and cancer groups. The Youden's index criterion was used to identify the “optimal” cutoff value for differentiating between normal controls and cancer groups. The sensitivity and specificity associated with the cutoff value were estimated using 95% credible regions obtained by Jeffreys prior method.

Cutoff value testing: Using the test cohort, an ROC curve and corresponding cross-validated AUC were generated based on the observed exosomal TF-Ag-α values. The cutoff value developed using the training cohort was applied, and the sensitivity and specificity were calculated.

### Example 6: Exosomal Thomsen-Friedenreich Glycoantigen as a Sensitive and Specific Biomarker for Colon, Ovarian and Prostate Cancer Diagnosis

The screening and diagnosis of colon, ovarian, and prostate cancers are challenged by invasive procedures and lack of effective screening tests. To address these challenges, we developed an exosome-based liquid biopsy assay that utilized a new exosomal biomarker, α-linked Thomsen-Friedenreich glycoantigen (TF-Ag-α, Galβ1-3GalNAc-α). This assay provides a minimally invasive method with high sensitivity and specificity for the screening and diagnosis of colon, ovarian and prostate cancers. Exosomal TF-Ag-α levels in 10 μL serum samples were measured using a compact, surface plasmon resonance-based biosensor developed to facilitate clinical translation. The diagnostic performance of exosomal TF-Ag-α was initially evaluated in a training set consisting of normal controls (n=80) and cancer patients (n=120, 40 for prostate, 40 for colon, and 40 for ovarian cancer), and then validated in an independent test set including normal controls (n=29) and cancer patients (n=60, 20 for each cancer type). Receiver operating characteristic (ROC) analysis was performed to assess diagnostic sensitivity, specificity and area under the curve (AUC) of exosomal TF-Ag-α. Serum samples from cancer patients showed significantly elevated levels of exosomal TF-Ag-α compared with normal controls. Exosomal TF-Ag-α distinguished all cancer patients from normal controls with a sensitivity of 99.2%, specificity of 100% and an overall accuracy of 99.5% at a cutoff value of 0.406. These findings demonstrated the clinical translational promise of exosomal TF-Ag-α as a liquid biopsy biomarker for the detection of colon, ovarian and prostate cancers.

### Introduction

Cancer remains the leading cause of death worldwide, posing a significant public health challenge. Many malignancies, such as lung and ovarian cancers, are often diagnosed at advanced stages, where treatment options are limited and five-year survival rates are poor. Early detection of cancer can dramatically improve outcomes, with survival rates increasing to 90% for some cancers. Therefore, early cancer detection is critical for improving patient prognosis and the effectiveness of treatment. However, current screening and early detection methods for major cancers have notable limitations. For colon cancer, diagnostic methods include colonoscopy, fecal occult blood tests (FOBT), fecal immunochemical tests (FIT), and stool DNA tests, with CT colonography used in some cases. Colonoscopy remains the gold standard, as it allows for direct visualization and biopsy of suspicious lesions. Nonetheless, it is invasive, requires bowel preparation and sedation, and carries a small risk of complications. Stool-based tests, while less invasive, may miss early or precancerous lesions and often require follow-up colonoscopy if positive. In the case of ovarian cancer, diagnosis typically relies on pelvic exams, transvaginal ultrasound, CA125 blood tests, and imaging such as CT scans, with definitive diagnosis often requiring surgical exploration. However, symptoms of ovarian cancer are often nonspecific and usually present at a late stage; CA125 can be elevated in benign conditions; imaging is limited in detecting early-stage disease; and no effective screening test exists for the general population. For prostate cancer, diagnostic approaches include prostate-specific antigen (PSA) blood tests, digital rectal exams (DRE), imaging (such as MRI), and confirmatory prostate biopsy. PSA testing lacks specificity, leading to overdiagnosis and unnecessary biopsies; DRE is operator-dependent and may miss early tumors; and imaging and biopsy can be invasive. New diagnostic approaches are urgently needed to overcome these limitations and enable earlier, more accurate detection across these cancer types.

One promising new approach to address these limitations is liquid biopsy, a minimally or non-invasive technique that analyzes tumor-derived materials—such as circulating tumor DNA (ctDNA), RNA, proteins, or exosomes—in bodily fluids like blood, urine, or saliva. Liquid biopsy offers several advantages over traditional methods: it is less invasive, can be performed repeatedly to monitor disease progression or treatment response, and has the potential to detect cancers at earlier stages. Among the various components analyzed in liquid biopsies, tumor-derived exosomes have emerged as potent biomarkers for cancer liquid biopsy. Exosomes are small extracellular vesicles (sEVs) secreted by cells which carry molecular cargos, such as DNA, RNA, and proteins that reflect the physiological or pathological state of their cells of origin. Because they are abundant in biofluids and contain rich molecular information, exosomes hold great promise for improving the sensitivity and specificity of cancer liquid biopsy across a wide range of cancer types, including colon, prostate, and ovarian cancers. For example, in colon cancer, elevated levels of exosomal miR-125a-3p in plasma were shown to distinguish early-stage patients from healthy individuals, with diagnostic accuracy improving when combined with carcinoembryonic antigen (CEA). For prostate cancer, tumor-derived exosomal av$3 integrin has been linked to aggressive disease and may serve as a non-invasive marker for monitoring progression. In ovarian cancer, plasma-derived exosomal miR-4732-5p was significantly upregulated in patients, suggesting its utility as a promising biomarker for early detection. These studies highlight exosome-based liquid biopsy as a powerful analytical assay capable of enabling minimally invasive and accurate cancer diagnosis.

Among various exosomal biomarkers, the Thomsen-Friedenreich glycoantigen (TF-Ag) has emerged as a promising candidate for cancer liquid biopsy. TF-Ag is the disaccharide D-galactose-β-(1-3)-N-acetylgalactosamine (Gal-β-(1-3)-GalNAc), which is typically α-linked to serine or threonine residues in normal tissues, but present in normal tissue as a more complex structure, either mono- or di-sialylated or otherwise further glycosylated by an N-acetylglucosamine addition to the galactose or by fucosylation. However, in tumor tissues, due to alterations in the glycosylation machinery, this disaccharide is present without further glycosylation. This tumor-associated form, TF-Ag-α, is therefore a promising cancer-specific biomarker. In our previous study, we developed a TF-Ag-α-specific monoclonal antibody, JAA-F11, and demonstrated TF-Ag-α expression across a range of lethal human carcinomas, including breast, lung, colon, bladder, ovarian, prostate, and stomach cancers. We further confirmed that TF-Ag-α is carried by exosomes and that serum exosomal TF-Ag-α could discriminate lung and breast cancers from normal controls with diagnostic accuracy of ≥95% and ≥97%, respectively. Building on these findings, we aimed to evaluate the diagnostic potential of exosomal TF-Ag-α in colon, ovarian, and prostate cancers. Exosomal TF-Ag-α levels in serum samples were measured using a compact surface plasmon resonance (SPR) biosensor, designed to provide highly sensitive and rapid (~1 h) detection of exosomal biomarkers with minimal sample volume (as little as 10 μL of serum) and simple operation requiring only a micropipette. Exosomal TF-Ag-αlevels were significantly higher in serum samples from patients with all examined cancer types (n=60 for prostate, n=60 for colon, and n=60 for ovarian cancer) compared to normal controls (n=109), achieving an overall accuracy of 99.5% at the cutoff value of 0.406. When combined with data from lung and breast cancer patients from our previous study, exosomal TF-Ag-α maintained a high discriminative accuracy of 96.2% across all five cancer types (prostate, colon, ovarian, lung, and breast), effectively distinguishing cancer patients (n=335) from non-cancer controls (n=187). These findings highlight the broad clinical utility of exosomal TF-Ag-α as a liquid biopsy biomarker for multi-cancer detection.

### Materials and Methods

Materials: Thiolated biotin polyethylene glycol 1000 (Biotin-PEG1000-SH, MW 1000 Da, PG2-BNTH-1k) was purchased from Nanocs (New York, NY, USA). Methyl-PEG4-thiol (PEG200-SH, MW 224.32 Da, 26132), NeutrAvidin protein (31000), EZ-Link Micro Sulfo-NHS-LC-Biotinylation kit (21935), and glass microscope slides (12-550-A3) were purchased from Thermo Fisher Scientific (Waltham, MA, USA). JAA-F11 mouse antibodies (anti-TF-Ag-α mAb) were supplied by For-Robin, Inc (Buffalo, NY, USA). Mouse IgG3 antibodies (0105-01, RRID: AB_2793898) were purchased from SouthemBiotech (Birmingham, AL, USA).

Human Serum Sample Collection and Processing: Serum samples and corresponding clinical data were provided by the Data Bank and BioRepository Shared Resource at Roswell Park Comprehensive Cancer Center (Buffalo, NY, USA). Serum samples were collected from normal controls and treatment-naïve patients with colon, ovarian, or prostate cancer. The clinical data of all participants were de-identified. All participants provided informed consent prior to sample collection. All groups were matched on age, gender and race/ethnicity distributions. Serum samples were subsequently transferred to the University at Buffalo and stored at −80 °C. Prior to testing, serum samples were centrifuged at 10,000 g for 30 min at 4 °C to remove debris. The use of human serum samples in this study was approved by the Institutional Review Boards of Roswell Park Comprehensive Cancer Center and the University at Buffalo.

Fabrication of SPR Biochip: Briefly, glass slides were cleaned by sequential 10 min sonication in acetone, 200 proof ethanol, and deionized water. After cleaning, the slides underwent Ti (2 nm, 0.8 Å/s) and Au (49 nm, 0.8 Å/s) deposition by using an electron-beam evaporator (Kurt J. Lesker Company, Jefferson Hills, PA, USA). The biochip was assembled by bonding an Au-coated glass slide to a PDMS sheet with a 6 mm-diameter sample well after oxygen plasma treatment (forward RF power was 15 W, reflected power was 0 W, for 75 s).

Surface Modification of SPR Biochip: A 10 mmol/L thiolated polyethylene glycol (PEG) mixture was prepared by mixing Biotin-PEG1000-SH with PEG200-SH at a molar ratio of 1:3. The biochip surface was then coated with PEG via thiol-gold interactions by incubating the PEG mixture (100 μL) on the surface for 1 h at room temperature. After excess PEG was washed off using PBS, 100 μL of 50 μg/mL NeutrAvidin was added and immobilized on PEGylated biochip following 1 h incubation at room temperature. Finally, unbound NeutrAvidin was washed off with PBS, and 100 μL of 50 μg/mL biotinylated mouse JAA-F11 or mouse IgG3 antibodies was added and conjugated on the surface of biochip through the biotin-avidin interaction. The biochip was incubated at 4 °C overnight for future use.

Detection of Exosomal TF-Ag-α by the SPR Biosensor: The modified biochip was immobilized on the prism of the SPR biosensor. A 647 nm laser with a power density of 30 mW was directed through the sample well at the SPR angle. To measure the expression of exosomal TF-Ag-α, deionized water and PBS were first applied to the biochip, and the intensity of the reflected laser beam (I) was measured using a photodetector as baseline measurement. After acquiring the SPR signal for 2 min with both water (I_{water}) and PBS (I_{PBS}), 50 μL of diluted human serum sample (10 μL serum+40 μL PBS) was added on the biochip and incubated for 1 h at room temperature. The TF-Ag-α-positive exosomes were captured by JAA-F11 antibodies, and the change in SPR signal was recorded. After washing off unbound exosomes, the SPR signal of TF-Ag-α-positive exosomes (I_{exosomal TF-Ag-α}) was recorded for 2 min. The same sample was also applied on an IgG3 antibody modified biochip, which was used as a control for the measurement of expression of exosomal TF-Ag-α to remove background noise caused by nonspecific binding. The expression level of exosomal TF-Ag-α was calculated using the following equation:

$$Expression of TEX TF - Ag - α = {( \frac{{I}_{TEX TF - Ag - α} - {I}_{PBS}}{{I}_{PBS} - {I}_{water}} )}_{JAA - F 11 antibody modified biochip} - {( \frac{{I}_{TEX TF - Ag - α} - {I}_{PBS}}{{I}_{PBS} - {I}_{water}} )}_{IgG 3 control antibody modified biochip}$$

The difference between I_{PBS} and I_{water} was used as the normalization factor to address the chip-to-chip variation. Although each biochip was prepared using the same procedure, factors such as the thickness of gold film can vary slightly, which may affect the measured exosomal TF-Ag-α signals. While individual biochips may differ and the absolute values of I_{PBS} and I_{water} may vary, their difference remains consistent, reflecting the refractive index difference between water and PBS, and thus serves as the normalization factor to correct for chip-to-chip variation.

Characterization of Exosomes by Nanoparticle Tracking Analysis: The size, size distribution, and number concentration of exosomes isolated from patient serum samples were characterized using a nanoparticle tracking analysis (NTA) system (NanoSight, LM10; Malvern Panalytical, Westborough, MA, USA). Exosomes were first isolated from 100 μL of patient serum samples using a total exosome isolation kit (from serum; 4478360; Thermo Fisher Scientific), following the manufacturer's instructions. The isolated exosomes were then diluted in PBS to achieve 50-100 nanoparticles within the field of view. All measurements were performed under identical instrument settings. Camera level, detection threshold, and screen gain were set at 14, 6, and 8, respectively, for view-capturing and particle tracking process.

Characterization of TF-Ag-α-Positive Tumor-Derived Exosomes: We employed scanning electron microscopy (SEM) to observe TF-Ag-α-positive, tumor-derived exosomes captured on the biochips. Briefly, after TF-Ag-α-positive exosomes were captured by JAA-F11 antibodies, unbounded exosomes were washed off using PBS. Then, the biochips were cleaned with deionized water and dried at 4 °C. Prior to SEM imaging, a layer of carbon was applied on the top of biochip, and TF-Ag-α-positive exosomes were characterized using a field-emission scanning electron microscope (SU-70; Hitachi High Technologies, Tokyo, Japan) with an energy-dispersive X-ray spectrometer.

Statistical Data Analysis: Receiver operating characteristic (ROC) analysis was performed using the SPSS software (Version 30; IBM, Armonk, NY, USA) to evaluate the discriminatory ability of exosomal TF-Ag-α between normal controls and cancer patients. In the training cohort, a logistic regression model was applied to generate the ROC curve, from which the area under the curve (AUC) was calculated and the optimal cutoff value was determined by the maximum Youden index (Youden Index=Sensitivity+Specificity−1).

To assess diagnostic performance at the identified cutoff, a binary classification variable was created, and diagnostic metrics including sensitivity, specificity, positive predictive value (PPV), negative predictive value (NPV), and overall accuracy (ACC) were computed using the Crosstabs function in SPSS, based on the confusion matrix. The sensitivity was defined as the proportion of true positive cases among all actual positives, and specificity as the proportion of true negative cases among all actual negatives. PPV was calculated as the proportion of true positive cases among all test-positive samples, while NPV represented the proportion of true negative cases among all test-negative samples. ACC was calculated as the proportion of correctly classified cases (true positives and true negatives) among all samples.

The cutoff value identified from the training cohort was then applied to an independent test cohort. ROC analysis of the test cohort was performed using the observed exosomal TF-Ag-α values to generate the cross-validated AUC, and diagnostic metrics were calculated to evaluate classification performance.

### Results

SPR-Based Detection and Characterization of Exosomal TF-Ag-α in Cancer Patient Serum: We employed a SPR-based liquid biopsy assay to assess the expression levels of exosomal TF-Ag-α in clinical serum samples from patients with prostate, colon, and ovarian cancers. The assay employed a compact SPR biosensor engineered for highly sensitive and fast (~1 h) detection of exosomal biomarkers. It requires only a small sample volume (as low as 10 μL of serum) and features a user-friendly design that can be operated with just a micropipette. In brief, the SPR assay detects changes in the local refractive index. As shown in FIG. 15A, a laser beam is directed through a prism onto a gold-coated biochip, where JAA-F11 antibodies, specific for TF-Ag-α, are immobilized on the gold surface. Upon the addition of serum (10 μL serum diluted in 40 μL PBS) on the biochip, only exosomes expressing TF-Ag-α bind to the antibody-coated surface, inducing a shift in the refractive index. This change is then detected by a photodetector as a measurable SPR signal. Representative SPR sensing curves for detecting exosomal TF-Ag-α in serum samples from prostate, colon and ovarian cancer patients are shown in FIGS. 15B-15D. To verify exosome capture on the biochips, SEM was performed. Representative SEM images (FIGS. 15E-15G) confirmed the presence of exosomes from serum samples of prostate, colon, and ovarian cancer patients. The average size of exosomes on the biochips was 82.41 nm, which agreed with results from nanoparticle tracking analysis (FIGS. 16A-16C).

Evaluation of Exosomal TF-Ag-α in Colon Cancer Diagnosis: To evaluate the diagnostic performance of exosomal TF-Ag-α in colon cancer, a training set comprising patients with colon cancer (n=40) and normal controls (n=40) was established. The colon cancer group had all four stages of colon cancer (n=10 at each stage). The normal control group included 20 low-risk individuals and 20 patients with benign colon conditions at high risk of colon cancer. The benign conditions included tubular adenoma, tubulovillous adenoma, villous adenoma, mucinous cystadenoma, diverticulosis, familial adenomatous polyposis, Gardner syndrome, abdominal desmoid tumors, and hereditary nonpolyposis colorectal cancer (HNPCC). Patient characteristics are summarized in TABLE 7 and TABLE 10. The exosomal TF-Ag-α levels were significantly higher (p<0.0001) in all colon cancer patients compared to both healthy individuals and those with benign colon conditions (FIG. 17A). No significant difference was observed between healthy and benign control groups, nor among the different cancer stages, indicating that exosomal TF-Ag-α expression is not correlated with tumor burden or disease stage.

TABLE 7
Characteristics of colon cancer patients and normal controls.
Characteristics n (%)

| | Training set | | Test set | |
|---|---|---|---|---|
| Total patients | 80 | | 49 | |
| Normal controls | 20 (25) | | 29 (59) | |
| (low risk) | | | | |
| Normal controls | 20 (25) | | 0 (0) | |
| (high risk) | | | | |
| Cancer | 40 (50) | | 20 (41) | |
| | Normal controls | | | |
| | (both low-risk and | Patients with | Normal controls | Patients with |
| | high-risk patients) | colon cancer | (low-risk patients) | colon cancer |

Gender

| | | | | |
|---|---|---|---|---|
| Female | 20 (25) | 20 (25) | 16 (33) | 10 (20) |
| Male | 20 (25) | 20 (25) | 13 (27) | 10 (20) |

Age (years)

| | | | | |
|---|---|---|---|---|
| Mean | 60 | 65 | 64 | 61 |
| Median | 60 | 66 | 64 | 58 |
| Range | 30-79 | 44-91 | 50-79 | 45-79 |

Stage

| | | |
|---|---|---|
| I | 10 (25) | 5 (25) |
| II | 10 (25) | 5 (25) |
| III | 10 (25) | 5 (25) |
| IV | 10 (25) | 5 (25) |

The diagnostic performance of exosomal TF-Ag-α in colon cancer was first evaluated by ROC analysis. Exosomal TF-Ag-α effectively distinguished colon cancer patients (n=40) from normal controls (n=40) with 97.5% sensitivity, 100% specificity, and an AUC of 0.999 at a cutoff value of 0.397 (FIG. 17B). Additionally, confusion matrix analysis showed 97.5% sensitivity, 100% specificity, 100% PPV, 97.6% NPV, and 98.7% ACC at the cutoff value of 0.397, confirming the diagnostic performance (FIG. 18A). When stratified by stage, as shown in FIGS. 18B-18C, exosomal TF-Ag-α distinguished early-stage (I/II) colon cancer patients (n=20) from controls with 95% sensitivity, 100% specificity, and an AUC of 0.998 at a cutoff value of 0.397. For late-stage (III/IV) patients (n=20), both sensitivity and specificity reached 100%, with an AUC of 1.00 at a cutoff value of 0.442.

The diagnostic performance of exosomal TF-Ag-α in colon cancer was further validated in a blinded, independent test set analyzed by a different operator than the one who handled the training cohort. The test set included 20 colon cancer patients and 29 normal controls. Patient characteristics are provided in TABLE 7 and TABLE 10. As shown in FIG. 17C, exosomal TF-Ag-α levels were significantly higher (p<0.0001) in serum samples from colon cancer patients compared to normal controls. Based on the observed exosomal TF-Ag-α levels, ROC analysis yielded a sensitivity of 100%, specificity of 100% and AUC of 1.00 (FIG. 17D). Using the cutoff value of 0.397 derived from the training set, exosomal TF-Ag-α achieved 100% sensitivity, specificity, AUC, PPV, NPV, and ACC in distinguishing colon cancer patients (stages I-IV) from controls, surpassing the performance metrics observed in the training cohort (FIGS. 18D-18F). These results not only validated exosomal TF-Ag-α as a highly sensitive and specific biomarker for colon cancer diagnosis but also demonstrated the ease of use and operator-friendliness of the SPR assay, supporting its strong potential for clinical application.

Diagnostic Evaluation of Exosomal TF-Ag-α in Ovarian Cancer: The diagnostic value of exosomal TF-Ag-α in ovarian cancer was first assessed using a training set consisting of ovarian cancer patients (n=40) and normal controls (n=30). The ovarian cancer group included 10 patients at each stage (stage I-IV), and the normal control group included 10 patients at low risk and 20 patients with benign ovarian conditions at high risk of ovarian cancer. The benign conditions included mucinous cystadenoma, serous cystadenoma, endometrioma, corpus luteum cyst, serous cyst, dermoid cyst, and follicular cyst. The patient characteristics are provided in TABLE 8 and TABLE 11. Significantly higher levels of exosomal TF-Ag-α were detected (p<0.0001) in ovarian cancer patients compared with low-risk controls and patients with benign ovarian conditions (FIG. 19A). No significant difference in exosomal TF-Ag-α levels was observed between low-risk individuals and patients with benign ovarian conditions, and between early and late stages.

TABLE 8
Characteristics of ovarian cancer patients and normal controls.
Characteristics n (%)

| | Training set | | Test set | |
|---|---|---|---|---|
| Total patients | 70 | | 36 | |
| Normal controls | 10 (14) | | 16 (44) | |
| (low risk) | | | | |
| Normal controls | 20 (29) | | 0 (0) | |
| (high risk) | | | | |
| Cancer | 40 (57) | | 20 (56) | |
| | Normal controls | | | |
| | (both low-risk and | Patients with | Normal controls | Patients with |
| | high-risk patients) | ovarian cancer | (low-risk patients) | ovarian cancer |

Gender

| | | | | |
|---|---|---|---|---|
| Female | 30 (43) | 40 (57) | 16 (44) | 20 (56) |

Age (years)

| | | | | |
|---|---|---|---|---|
| Mean | 58 | 65 | 64 | 59 |
| Median | 57 | 65 | 66 | 60 |
| Range | 38-85 | 40-89 | 50-79 | 47-76 |

Stage

| | | |
|---|---|---|
| I | 10 (25) | 5 (25) |
| II | 10 (25) | 5 (25) |
| III | 10 (25) | 5 (25) |
| IV | 10 (25) | 5 (25) |

ROC analysis was conducted to evaluate the sensitivity, specificity, and AUC of exosomal TF-Ag-α in diagnosing ovarian cancer (FIG. 19B). Exosomal TF-Ag-α successfully distinguished ovarian cancer patients (n=40) from normal controls (n=30) with 100% sensitivity, 100% specificity and AUC of 1.00 at cutoff value of 0.428. Confusion matrix analysis showed that the sensitivity, specificity, PPV, NPV, and ACC were 100% at the cutoff value of 0.428 (FIG. 20A). Exosomal TF-Ag-α also accurately differentiated both early-stage (I/II, n=20) and late-stage (III/IV, n=20) ovarian cancer patients from controls, with 100% sensitivity, 100% specificity and AUC of 1.00 at cutoff values of 0.428 and 0.437, respectively (FIGS. 20B-20C).

The diagnostic performance of exosomal TF-Ag-α in ovarian cancer was validated using a blinded, independent test cohort consisting of 20 ovarian cancer patients and 16 normal controls. Patient characteristics are detailed in TABLE 8 and TABLE 11. As shown in FIG. 19C, exosomal TF-Ag-α levels were significantly elevated in serum samples from ovarian cancer patients compared to normal controls (p=0.0002). Based on the observed exosomal TF-Ag-α levels, ROC analysis yielded a sensitivity, specificity, and AUC of 1.00 (FIG. 19D). When applying the cutoff value of 0.428 established from the training set, exosomal TF-Ag-α achieved 100% sensitivity, specificity, AUC, PPV, NPV, and ACC in distinguishing ovarian cancer patients (stages I-IV) from normal controls (FIGS. 20D-20F). Results from both the training and test cohorts demonstrated that exosomal TF-Ag-α serves as a highly sensitive and specific biomarker for ovarian cancer diagnosis.

To evaluate whether exosomal TF-Ag-α outperforms the established biomarker CA125 in ovarian cancer diagnosis, we compared their diagnostic performance. Pre-treatment CA125 data were available for a subset of ovarian cancer patients (n=27) and patients with benign conditions (n=3), with blood samples collected within 100 days of those used for exosomal TF-Ag-α analysis to maintain temporal and analytical consistency. As shown in FIGS. 21A-21H, at a cutoff value of 30.2 U/mL, CA125 demonstrated limited diagnostic accuracy, with a sensitivity of 74.1%, specificity of 33.3%, AUC of 0.728, NPV of 12.5%, PPV of 90.9%, and ACC of 70%. Among the 27 ovarian cancer patients, 18 were diagnosed at an early stage. The diagnostic performance of CA125 further declined when distinguishing early-stage ovarian cancer from benign conditions, yielding a sensitivity of 61.1%, specificity of 33.3%, AUC of 0.630, NPV of 12.5%, PPV of 84.6%, and ACC of 57.1%. In contrast, exosomal TF-Ag-α achieved perfect diagnostic performance in detecting ovarian cancer, including both early- and late-stage cases, with a sensitivity, specificity, AUC, NPV, PPV, and ACC all equal to 100%.

Evaluation of Exosomal TF-Ag-α in Prostate Cancer Diagnosis: To evaluate the diagnostic performance of exosomal TF-Ag-α in prostate cancer, we first established a training set consisting of 40 prostate cancer patients and 30 normal controls. The prostate cancer group included 20 early-stage (I/II) and 20 late-stage (III/IV) patients. The normal control group included 10 patients at low risk and 20 patients with benign prostate conditions at high risk of prostate cancer. The benign conditions included benign prostatic hyperplasia (BPH), high-grade prostatic intraepithelial neoplasia (HGPIN, PIN III), focal glandular hyperplasia, benign phyllodes tumor, and prostatitis. The patient characteristics are provided in TABLE 9 and TABLE 12. Exosomal TF-Ag-α levels were markedly elevated (p<0.0001) in serum samples from prostate cancer patients (n=40) compared to normal controls (n=30) (FIG. 22A). No significant difference was observed between low-risk controls (n=10) and patients with benign prostate conditions (n=20). Consistent with the findings in colon and ovarian cancers, exosomal TF-Ag-α levels did not differ significantly across prostate cancer stages, indicating that this biomarker is not associated with tumor progression.

TABLE 9
Characteristics of prostate cancer patients and normal controls.
Characteristics n (%)

| | Training set | | Test set | |
|---|---|---|---|---|
| Total patients | 70 | | 33 | |
| Normal controls | 10 (14) | | 13 (39) | |
| (low risk) | | | | |
| Normal controls | 20 (29) | | 0 (0) | |
| (high risk) | | | | |
| Cancer | 40 (57) | | 20 (61) | |
| | Normal controls | | | |
| | (both low-risk and | Patients with | Normal controls | Patients with |
| | high-risk patients) | prostate cancer | (low-risk patients) | prostate cancer |

Gender

| | | | | |
|---|---|---|---|---|
| Male | 30 (43) | 40 (57) | 13 (39) | 20 (61) |

Age (years)

| | | | | |
|---|---|---|---|---|
| Mean | 64 | 60 | 63 | 62 |
| Median | 64 | 60 | 61 | 59 |
| Range | 51-78 | 45-81 | 51-77 | 53-74 |

Stage

| | | |
|---|---|---|
| I | 10 (25) | 5 (25) |
| II | 10 (25) | 5 (25) |
| III | 10 (25) | 5 (25) |
| IV | 10 (25) | 5 (25) |

ROC analysis revealed that exosomal TF-Ag-α could accurately discriminate prostate cancer patients (n=40) from normal controls (n=30), achieving 100% sensitivity, 100% specificity and an AUC of 1.00 at the cutoff value of 0.487 (FIG. 22B). Confusion matrix analysis showed that the sensitivity, specificity, PPV, NPV, and ACC all reached 100% at the cutoff value of 0.487 (FIG. 23A). To further refine our assessment, prostate cancer patients were stratified into early-stage (stage I/II, n=20) and late-stage (stage III/IV, n=20) groups. Both groups were distinguished from normal controls with a sensitivity of 100%, specificity of 100% and AUC of 1.00 at cutoff values of 0.514 and 0.487, respectively (FIGS. 23B-23C). These findings demonstrated exosomal TF-Ag-α as a robust and potent biomarker for early-stage prostate cancer, capable of distinguishing prostate cancer patients from normal controls and those with benign prostate conditions.

The diagnostic performance of exosomal TF-Ag-α was validated in a blinded, independent test set of 20 prostate cancer patients (10 stage I/II and 10 state III/IV) and 13 normal controls (TABLE 9 and TABLE 12). To assess reproducibility, measurements were independently performed by three operators. As shown in FIG. 22C, serum exosomal TF-Ag-α levels were markedly higher in prostate cancer patients than in normal controls (p<0.0001). ROC analysis of observed exosomal TF-Ag-α levels was conducted to compare overall prostate cancer patients versus normal controls, as well as early-stage (I/II) and late-stage (III/IV) subgroups versus normal controls (FIG. 22D, FIGS. 23E-23F). In all three comparisons, the sensitivity and specificity were 100% and the AUC was 1.00, identical to results from the training cohort, demonstrating robust diagnostic accuracy. When the cutoff value of 0.487 identified from the training set was applied, exosomal TF-Ag-α achieved 100% sensitivity, specificity, AUC, PPV, NPV and ACC in distinguishing prostate cancer patients (stages I-IV) from normal controls (FIG. 23D). Notably, one sample from a stage I patient in test cohort, collected 75 days prior to clinical diagnosis, was correctly classified, further emphasizing the biomarker's potential in early prostate cancer detection. These results demonstrate that exosomal TF-Ag-α is an ultra-sensitive and specific biomarker for prostate cancer detection, showing consistent performance across training and test cohorts.

To demonstrate that exosomal TF-Ag-α outperforms the conventional biomarker PSA in prostate cancer diagnosis, we compared their diagnostic performance. Pre-treatment PSA data were available for a subset of prostate cancer patients (n=23) and patients with benign conditions (n=5), with blood samples obtained within 100 days of those used for exosomal TF-Ag-α analysis to ensure comparability. As shown in FIGS. 24A-24H, at a cutoff value of 4.0 ng/mL, PSA demonstrated poor diagnostic accuracy, with a sensitivity of 34.8%, specificity of 40.0%, AUC of 0.304, NPV of 11.8%, PPV of 72.7%, and ACC of 35.7%. When limited to early-stage prostate cancer cases (n=8), PSA performance further deteriorated, with a sensitivity of 12.5%, specificity of 40.0%, AUC of 0.05, NPV of 22.2%, PPV of 25.0%, and ACC of 23.1%. In contrast, exosomal TF-Ag-α demonstrated outstanding diagnostic accuracy across both early- and late-stage prostate cancer, achieving 100% sensitivity, specificity, AUC, NPV, PPV, and ACC.

Evaluation of Exosomal TF-Ag-α Across Three Cancer Types (Colon, Ovarian, and Prostate): After confirming the diagnostic performance of exosomal TF-Ag-α in detecting individual cancer types (prostate, colon, and ovarian cancer), we evaluated its diagnostic value across all three cancer types. We pooled the training sets for each cancer type, resulting in a combined cohort of 200 cancer patients and normal controls. As shown in FIG. 25A, exosomal TF-Ag-α levels were significantly higher in cancer patients (n=120) compared to non-cancer controls (n=80; n=20 at low risk, n=60 at high risk) (p<0.0001). Exosomal TF-Ag-α maintained high discriminative accuracy, yielding an AUC of 0.999 with 99.2% sensitivity and 100% specificity at a cutoff value of 0.406, as determined by ROC analysis (FIG. 25B). When the cutoff value of 0.406 was applied for binary classification, confusion matrix analysis confirmed the diagnostic performance, yielding 99.2% sensitivity, 100% specificity, 100% PPV, 98.8% NPV, and 99.5% ACC (FIG. 26A). Exosomal TF-Ag-α distinguished normal controls from early-stage (I-II) cancer patients with 98.3% sensitivity, 100% specificity and an AUC of 0.999 at a cutoff value of 0.406, and from late-stage (III-IV) cancer patients with 100% sensitivity, 100% specificity and an AUC of 1.00 at a cutoff value of 0.448 (FIGS. 26B-26C).

Pooled analysis of all three cancer test cohorts (n=89) confirmed significantly elevated exosomal TF-Ag-α levels in cancer patients (n=60) compared with controls (n=29) (p<0.0001), with an AUC of 1.00, sensitivity of 100%, specificity of 100%, PPV of 100%, NPV of 100%, and ACC of 100% at a cutoff value of 0.406, outperforming the diagnostic performance observed in the training set (FIGS. 25C-25D, FIGS. 26D-26F). Importantly, exosomal TF-Ag-α maintained high diagnostic accuracy in early-stage patients, underscoring its potential as a promising biomarker for early cancer detection.

Evaluation of Exosomal TF-Ag-α Across Five Cancer Types (Colon, Ovarian, Prostate, Lung and Breast): Building on previous findings in lung and breast cancers, pooled data from five cancer types (prostate, colon, ovarian, lung, and breast; total n=522) were analyzed to assess the overall diagnostic performance of exosomal TF-Ag-α. Exosomal TF-Ag-α levels were significantly higher in cancer patients (n=335; stage 0/1/II: n=174; stage III/IV: n=161) compared to non-cancer controls (n=187; low risk: n=89; benign condition at high risk: n=98) (p<0.0001; FIG. 27A). Exosomal TF-Ag-α achieved high discriminative accuracy, with an AUC of 0.980, sensitivity of 97.0%, specificity of 94.7%, PPV of 97.0%, NPV of 94.7%, and ACC of 96.2% in distinguishing all cancer patients from normal controls at a cutoff value of 0.407 (FIG. 27B, FIG. 28A). Exosomal TF-Ag-α analysis achieved high detection accuracy, with an AUC of 0.981, sensitivity of 97.6% and specificity of 94.7% in distinguishing early-stage cancer patients from normal controls at a cutoff value of 0.407, which underscores the potential of exosomal TF-Ag-α as an early cancer detection biomarker (FIG. 28B). Similarly, exosomal TF-Ag-α discriminated late-stage cancer patients from normal controls with an AUC of 0.979, sensitivity of 96.3% and specificity of 94.7% (FIG. 28C).

When analyzed by cancer type, a significant difference in exosomal TF-Ag-α levels was observed only between breast cancer and colon cancer (p=0.0071), but not among other cancer types (FIG. 29A). No significant differences were detected across cancer stages (FIGS. 29B-29C). These findings indicate that exosomal TF-Ag-α levels remain largely consistent across cancer stages and exhibit only limited variation among tumor types. Unlike tissue-specific biomarkers that are limited to detecting individual cancer types, exosomal TF-Ag-α demonstrates consistent overexpression across diverse tumor origins. When combined with the high sensitivity, specificity, and label-free, real-time detection capabilities of SPR-based biosensors, this single-marker approach offers a promising platform for universal cancer screening independent of tissue origin.

### Discussion

The United States Preventive Services Task Force (USPSTF) currently recommends screening tests for only four cancer types: low-dose CT scans for lung cancer, mammography for breast cancer, cytology/HPV testing for cervical cancer, and colonoscopy/stool-based testing for colorectal cancer. While these screening modalities have demonstrated population-level benefits, they are constrained by significant limitations, including suboptimal sensitivity and specificity, invasive procedures, radiation exposure, and high cost. Moreover, many malignancies with lower prevalence rates, such as pancreatic and ovarian cancers, currently lack effective screening options. This underscores an urgent need to develop highly sensitive, specific, and cost-effective screening methodologies for early cancer detection. Liquid biopsy platforms have emerged as particularly promising solutions, offering minimally invasive alternatives through the analysis of tumor-derived biomarkers in blood, urine, or other biofluids. Such approaches could potentially overcome key limitations of current methods by eliminating invasive procedures, reducing preparation complexity, and improving patient compliance.

Conventional blood-based biomarkers such as CA125 (ovarian cancer) and PSA (prostate cancer) remain in clinical use, yet their effectiveness for early detection is fundamentally constrained by insufficient sensitivity and specificity. In contrast, exosomes represent a transformative biomarker class characterized by three biologically and clinically important properties: their active secretion from viable tumor cells, molecular cargos that recapitulate tumor biology, and abundant stable presence in circulation. These intrinsic features have established exosome-based diagnostics as a compelling platform across the cancer care continuum, from screening, early detection, treatment monitoring to prognosis.

We previously showed that TF-Ag-α is broadly expressed across multiple tumor types, including breast, lung, prostate, colon, bladder, and ovarian cancers but being absent in normal tissues, highlighting its diagnostic potential. We further demonstrated that TF-Ag-α is present on exosomes, enabling accurate discrimination of lung and breast cancer patients from normal controls. These findings inspired us to investigate the diagnostic utility of exosomal TF-Ag-α across additional cancer types. In the present study, we evaluated exosomal TF-Ag-α for the diagnosis of colon, ovarian, and prostate cancers using serum samples from 109 normal controls and 180 cancer patients. Employing our established SPR-based liquid biopsy platform with only 10 μL of serum per assay, we found that exosomal TF-Ag-α exhibited exceptional diagnostic performance, achieving 99.2% sensitivity, 100% specificity, and an AUC of 0.999 for distinguishing cancer patients from controls. Notably, exosomal TF-Ag-α maintained high detection capability for early-stage (I/II) cancers, with 98.3% sensitivity, 100% specificity, and an AUC of 0.999, demonstrating its strong potential for early cancer detection.

By integrating data from our previous and current studies (total n=522; 335 patients with lung, breast, colon, ovarian, or prostate cancer and 187 normal controls), exosomal TF-Ag-α demonstrated strong diagnostic performance for multi-cancer detection. It distinguished all cancer patients from normal controls with a sensitivity of 97%, specificity of 94.7%, AUC of 0.980, NPV of 94.7%, PPV of 97% and ACC of 96.2%.

Our analysis revealed no significant differences in exosomal TF-Ag-α levels across cancer stages, suggesting this biomarker is not indicative of tumor progression stage. This observation may reflect inherent tumor heterogeneity, as we also observed that TF-Ag-α expression in tumor tissues was independent of disease stage. Our findings align with prior research by Takanami et al., who demonstrated that TF-Ag expression showed no correlation with gender, tumor size, lymph node involvement, distant metastasis, disease stage, or tumor differentiation grade in pulmonary adenocarcinoma. However, TF-Ag immunoreactivity did exhibit significant prognostic value for overall survival.

Although exosomal TF-Ag-α levels did not differ significantly among cancer stages, this limitation does not diminish its clinical utility for cancer screening and early detection. For a screening or diagnostic biomarker, the primary objective is to distinguish cancer from non-cancer individuals, particularly at early stages when intervention is most effective. In our subgroup analysis, exosomal TF-Ag-α demonstrated robust discrimination between stage 0/1/II cancers and normal controls, achieving an AUC of 0.981, sensitivity of 97.6%, and specificity of 94.7% across five cancer types. These findings indicate that exosomal TF-Ag-α holds great potential as a sensitive and reliable biomarker for cancer screening and early detection.

Among the five cancer types examined, only breast cancer exhibited significantly higher levels of exosomal TF-Ag-α compared to colon cancer, with no significant differences observed among the other cancers. These findings indicate that exosomal TF-Ag-α alone has limited ability to distinguish between cancer types. However, it can be incorporated into a multianalyte test alongside organ- or tumor type-specific biomarkers to enable simultaneous detection and localization of multiple cancer types. Moreover, its high sensitivity and specificity for early-stage disease make it an attractive candidate for inclusion in multimodal screening strategies. For example, exosomal TF-Ag-α may serve as a complementary blood test to low dose CT for lung cancer, as an adjunctive triage test for mammography in breast cancer, and as an alternative/triage modality alongside colonoscopy/stool-based testing for colorectal cancer.

Collectively, our results have demonstrated that exosomal TF-Ag-α could serve as a robust and broadly applicable biomarker for early multi-cancer detection. Moving forward, we will evaluate its diagnostic performance in additional cancer types, such as brain and bladder cancers. We plan to further validate the clinical utility of exosomal TF-Ag-α in large patient cohorts. For example, our comparison with PSA indicates that exosomal TF-Ag-α demonstrates substantially superior diagnostic performance for prostate cancer. However, this comparison was limited to a small subset of patients and lacked comprehensive clinical annotations, including Gleason scores, molecular data, and longitudinal outcomes. Thus, although exosomal TF-Ag-α appears more accurate than PSA for detecting prostate cancer, further studies are needed to determine whether it preferentially identifies higher-risk disease or reduces overtreatment of indolent tumors. Prospective studies incorporating detailed histopathologic and genomic information will be essential to evaluate whether exosomal TF-Ag-α correlates with tumor aggressiveness and clinical progression.

To assess its value for early cancer detection, we plan to collect blood samples from patients at various time points prior to diagnosis to determine how early exosomal TF-Ag-α can indicate the presence of cancer. We will also investigate whether exosomal TF-Ag-α can serve as a complementary assay to enhance the accuracy of existing screening and diagnostic methods. For example, in cases where low-dose CT detects an indeterminate nodule, measuring exosomal TF-Ag-α levels may help distinguish between benign and malignant findings. Additionally, combining exosomal TF-Ag-α with established markers such as CA125 for ovarian cancer and PSA for prostate cancer could improve diagnostic accuracy.

Given that elevated exosomal TF-Ag-α levels are observed in multiple cancer types, it may be used in conjunction with cancer-type- or organ-specific markers to develop a multi-cancer early detection (MCED) test. Furthermore, exosomal TF-Ag-α holds promise as a biomarker for monitoring treatment response, particularly for procedures involving organ removal. For instance, following mastectomy for breast cancer, few effective methods exist to monitor recurrence. Tracking changes in exosomal TF-Ag-α expression may offer a valuable approach for early relapse detection.

### Conclusions

In summary, we showed that exosomal TF-Ag-α was a highly sensitive and specific biomarker for colon, ovarian, and prostate cancers. The exosome-based liquid biopsy assay achieved 99.5% diagnostic accuracy across all three cancer types. This approach offers a simple, accurate, and minimally invasive alternative to current diagnostic procedures, with the potential to enhance current diagnostic practices, enable early cancer detection, and ultimately improve patient outcomes.

### Example 7: The Use of TF-Ag-α to Selectively Capture Tumor-Derived Exosomes And Enhance the Predictive Performance of Exosomal PD-L1 in Predicting Responses to Immune Checkpoint Inhibitors

Only a subset of non-small cell lung cancer (NSCLC) patients benefits from immune checkpoint inhibitors (ICIs). Tissue PD-L1 has limited accuracy in predicting ICI response and requires invasive biopsies, highlighting the urgent need for reliable, minimally invasive biomarkers. Exosomal PD-L1 is a promising liquid biopsy marker, but its predictive performance is confounded by PD-L1 derived from non-tumor cells. We developed a tumor-specific exosome-based liquid biopsy to predict ICI response. Tumor-derived exosomes (TEXs) were selectively captured using the tumor-specific Thomsen-Friedenreich glycoantigen alpha (TF-Ag-α), and PD-L1 levels in TEXs expressing TF-Ag-α (i.e., TEX^{TF-Ag-α} PD-L1) were measured in sera from 91 NSCLC patients receiving anti-PD-1 therapy (40 responders and 51 non-responders). Predictive performance was evaluated at pre-treatment baseline, at the end of first treatment cycle, and via fold changes. At pre-treatment baseline, TEX^{TF-Ag-α} PD-L1 showed moderate predictive ability (AUC: 0.76; sensitivity: 0.97; specificity: 0.56). By the end of the first treatment cycle, responders exhibited a ~4.18-fold increase in TEX^{TF-Ag-α} PD-L1 expression, whereas non-responders showed an approximate 20% decrease. Dynamic fold changes after the first treatment cycle substantially improved predictive performance, achieving an AUC of 0.93, sensitivity of 0.95, and specificity of 0.80 for distinguishing responders from non-responders, outperforming both total exosomal PD-L1 and tissue PD-L1. These results demonstrated that selective TEX capture using TF-Ag-α significantly enhanced the predictive value of exosomal PD-L1. Dynamic changes in TEX^{TF-Ag-α} PD-L1 provided a highly accurate, minimally invasive biomarker for early assessment of anti-PD-1 response, supporting tumor-specific exosome-based liquid biopsy for precision immunotherapy.

### Introduction

Lung cancer causes about 25% of all cancer deaths worldwide. While immune checkpoint inhibitors (ICI), such as pembrolizumab and nivolumab have revolutionized treatment for various cancers including non-small cell lung cancer (NSCLC), only a subset of patients receive durable clinical benefit. In the absence of predictive markers of response to ICI therapy, treatment choices are empiric and further complicated by unusual response patterns and potentially severe immune-related adverse effects (irAEs). Currently tissue biopsy and imaging modalities are used to predict and monitor the response to ICI therapy. Unfortunately, PD-L1 expression in pre-treatment tumor samples has a disappointing 28.9% accuracy in predicting response to ICI therapy. Repeated tumor biopsies may show increased T-cell clonality and expression of immune cytolytic markers in responding tumors, and would be ideal to evaluate evolution of tumor and microenvironment during ICI therapy. However, this approach is invasive and challenging for visceral tumors such as NSCLC. Imaging is non-invasive but limited by issues such as pseudo progression and delayed response. New tests are needed to address these limitations, effectively predict and monitor response to ICI therapy, and improve patient care and clinical outcomes.

Liquid biopsy enables the detection of tumor-derived biomarkers in body fluids such as blood and urine, making it possible to repeatedly assess disease status over time. By complementing medical imaging and clinical risk factor information, it can provide a broader view of tumor biology and progression, offering a non- or minimally invasive approach to help address key challenges in ICI therapy. Exosomes are nano-sized extracellular vesicles secreted by cells. They transport versatile cargos (such as proteins and microRNAs) between cells for intercellular communication. Tumor-derived exosomes (TEXs) play important roles in every step of cancer development, immune regulation, metastasis and drug resistance. Therefore, TEXs are promising biomarkers for cancer liquid biopsy in various applications spanning from cancer early detection to treatment response prediction.

Recent studies have shown that exosomal PD-L1 is closely correlated with responses to anti-PD-1/PD-L1 therapy. In melanoma patients, increased levels of exosomal PD-L1 at early stage of treatment have been observed in responders to anti-PD-1 therapy. This increase resulted from a tumor adaptive mechanism in which cancer cells release PD-L1-bearing exosomes that bind anti-PD-1 antibodies, thereby circumventing ICI therapy. In lung cancer patients, decreased levels of exosomal PD-L1 were observed in responders at a later stage of treatment (3-4 months post treatment. A recent meta-analysis study reported that increased exosomal PD-L1 levels in the early stages of immunotherapy was associated with improved patient prognosis. However, PD-L1 is expressed not only by cancer cells but also by immune cells, including T cells, B cells, NK cells, dendritic cells, and macrophages, all of which can release PD-L1-positive exosomes. Exosomal PD-L1 derived from non-tumor cells may therefore confound the detection of TEX PD-L1, reducing the predictive accuracy of PD-L1 as a biomarker for response to ICI therapy.

We recently discovered a tumor-specific exosomal biomarker, Thomsen-Friedenreich glycoantigen alpha (TF-Ag-α). TF-Ag is a disaccharide with chemical structure of D-galactose-β-(1-3)-N-acetylgalactosamine (Gal-β-(1-3)-GalNAc). TF-Ag is typically α-linked to serine or threonine residues on proteins. In normal tissues, TF-Ag is usually concealed by additional glycosylation, forming more complex structures that may be mono- or di-sialylated, extended through the addition of N-acetylglucosamine to galactose, or further modified by fucosylation. In tumor tissues, however, changes in the glycosylation machinery prevent these subsequent modifications, leaving TF-Ag exposed in its simpler form. This tumor-associated form, TF-Ag-α, therefore represents a promising cancer-specific biomarker. In our previous studies, we developed a TF-Ag-α-specific monoclonal antibody, JAA-F11, and demonstrated TF-Ag-α expression across a broad range of cancers, including lung, breast, colon, bladder, ovarian, prostate, and stomach cancers. We further confirmed that TF-Ag-α is carried by exosomes and showed that exosomal TF-Ag-α in serum samples effectively discriminated five cancer types (lung, breast, colon, ovarian, and prostate; n=335; stage 0/1/II: n=174; stage III/IV: n=161) from non-cancer controls (n=187; low risk: n=89; benign conditions at high risk: n=98), achieving a sensitivity of 97.0%, specificity of 94.7%, and overall accuracy of 96.2% at a cutoff value of 0.407.

In this study, TF-Ag-α was used to selectively capture TEXs from the total exosome population. The levels of TEX TF-Ag-α and TEX^{TF-Ag-α} PD-L1 in serum samples were then measured simultaneously using the Exo-PROS biosensor. Nonsmall cell lung cancer (NSCLC) patients receiving anti-PD-1 therapy (n=91), including 40 responders (complete response and partial response) and 51 non-responders (stable disease and progression), were enrolled in this study. Their serum samples were collected at pre-treatment baseline and at the end of first treatment cycle. The dynamic fold changes of TEX^{TF-Ag-α} PD-L1 distinguished responders from non-responders with a sensitivity of 0.95, specificity of 0.80 and an area under the curve (AUC) of 0.93 at the cutoff of 1.59. Notably, this study has demonstrated that TEX^{TF-Ag-α} PD-L1 exhibits superior sensitivity, specificity, and overall predictive accuracy compared with total exosomal PD-L1 and tissue PD-L1 in predicting response to anti-PD-1 therapy.

### Materials and Methods

Materials: Methyl-(PEG)4-thiol (PEG200-SH, MW 224 Da, 26132) was purchased from Sigma-Aldrich (St. Louis, MO). Biotin-PEG1000-SH (MW 1000 Da, PG2-BNTH-1k) was purchased from Nanocs (Boston, MA). NeutrAvidin protein (31000), biotinylated anti-PD-L1 antibody (clone MIH1, 13-5983-82, RRID:AB_466840), fluorescent dye APC labelled anti-PD-L1 antibody (clone MIH1, 17-5983-42, RRID:AB_10597586) and EZ-Link Micro Sulfo-NHS-LC-Biotinylation Kit (21935) were purchased from Thermo Fisher Scientific (Waltham, MA). Biotinylated anti-LG3BP antibody (LS-C686310-1) was purchased from LSBio (Seattle, WA). JAA-F11 anti-TF-Ag-α mouse antibody was obtained from For-Robin, Inc. (Buffalo, NY). ASYLGARD 184 silicone elastomer kit (PDMS, 2065622) was purchased from Dow Corning (Midland, MI).

Fabrication of Exo-PROS biochip: A glass slide (12-550-A3, Fisher Scientific) underwent cleaning using acetone, 200-proof ethanol, and DI water with 10 minutes of sonication. Using the Kurt J. Lesker AXXIS Electron-Beam Evaporator, a 2 nm layer of Ti followed by a 49 nm layer of Au was deposited onto the surface of the glass slide. The deposition rate was set at 0.8 Å/s for Ti and 1 Å/s for Au. Finally, a PDMS piece featuring a 6 mm diameter well and an Au-coated glass slide was subjected to oxygen plasma treatment and then securely bonded together for subsequent use.

Surface modification of Exo-PROS biochip: The surface of the Exo-PROS biochip was first modified with polyethylene glycol (PEG) by incubating it with a mixture of PEG200-SH and biotin-PEG1000-SH (100 μL, 10 mmol/L in PBS; PEG200-SH:biotin-PEG1000-SH molar ratio of 3:1) for 1 hour at room temperature (RT). Excess PEG was removed by washing three times with PBS. NeutrAvidin (100 μL, 50 μg/mL) was then added and incubated at RT for 1 hour. After removal of excess NeutrAvidin by three PBS washes, biotinylated antibodies against TF-Ag-α, PD-L1, or LG3BP (50 μL, 50 μg/mL) were added and incubated on the biochip overnight at 4 °C to immobilize the antibodies on the surface via biotin-avidin interactions. Excess antibodies were removed by washing three times with PBS, and the biochip was then ready for use.

Detection of TEX TF-Ag-α and TEX^{TF-Ag-α} PD-L1 using Exo-PROS assay: The biochip was mounted onto the Exo-PROS biosensor. A 647 nm laser with a power density of 30 mW was directed through the sample to generate surface plasmon resonance (SPR) signals. The SPR signals of water (I_{water}) and PBS (I_{PBS}) were recorded for 2 minutes to establish the baseline and minimize the chip-chip variation. Human serum samples (10 uL serum diluted with 40 uL PBS) were then applied to the biochip coated with anti-TF-Ag-α antibodies, and the SPR signal acquisition was performed at RT for 1 hour. Non-specific binding of exosomes was removed by washing three times with PBS. The SPR signal from TF-Ag-α positive exosomes (I_{TEX TF-Ag-α}) was subsequently recorded for 2 minutes. The expression level of TEX TF-Ag-α was calculated using the following equation.

TEX TF-Ag-α Expression=(I_{TEX TF-Ag-α}−I_{PBS})/I_{PBS}−I_{water})

To measure the level of TEX^{TF-Ag-α} PD-L1, an avalanche photodiode was positioned above the biochip, and the baseline fluorescence signal was recorded (F_{before APC-PD-L1}). Fluorescent dye APC-conjugated anti-PD-L1 antibodies (200 μg/mL, 5 μL) were then added and incubated on the biochip for 1.5 h at RT. After washing off the unbound APC-anti-PD-L1 antibodies, the fluorescence signal from TEX^{TF-Ag-α} PD-L1 was recorded (F_{after APC-PD-L1}). The expression level of TEX^{TF-Ag-α} PD-L1 was calculated using the following equation.

TEX^{TF-Ag-α}PD-L1 Expression=(F_{after APC-PD-L1}−F_{before APC-PD-L1})/F_{before APC-PD-L1}

The same procedure was used to detect TEX LG3BP and TEX^{LG3BPα} PD-L1 levels using a biochip modified with anti-LG3BP antibodies.

Detection of total exosomal PD-L1 using Exo-PROS assay: Similar to the detection of TEX TF-Ag-α, total exosomal PD-L1 was measured using a biochip modified with anti-PD-L1 antibodies. The expression level of total exosomal PD-L1 was calculated using the following equation.

Total TEX PD-L1 Expression=(I_{TEX PD-L1}−I_{PBS})/(I_{PBS}−I_{water})

Human serum samples: De-identified serum samples and clinical data of nonsmall cell lung cancer patients who received anti-PD-1 immunotherapy were obtained from the Data Bank and BioRepository Shared Resource at Roswell Park Comprehensive Cancer Center. After the serum samples were transferred to the University at Buffalo on dry ice, they were stored at −80 °C. All serum samples were centrifuged at 10,000 g at 4 °C for 30 minutes to remove debris and then diluted with PBS at a volume ratio of 1:4 before use. Approvals from Institutional Review Boards of Roswell Park Comprehensive Cancer Center and the University at Buffalo were obtained for the use of human serum samples in this study.

Statistical data analysis: Binary logistic regression and receiver operating characteristic (ROC) analyses were performed to compare responders and non-responders using individual biomarkers or their combinations in SPSS (Version 26, RRID:SCR_002865). For the PD-L1 tumor proportion score (TPS)-based ROC analysis, tissue PD-L1 TPS was dichotomized by classifying samples with TPS≥50% as responders and TPS<50% as non-responders, and this binary outcome was used for subsequent ROC evaluation. Cut-offs maximizing the sum of sensitivity and specificity were first identified, and the corresponding area under the curve (AUC), sensitivity, and specificity were then calculated at the selected cutoff. In the proportional stacked bar chart, patients were stratified into three predefined percentage categories (<1%, 1-49%, and ≥50%). For each cohort (responders and non-responders), the number of patients falling into each category was tabulated and entered into GraphPad Prism (Version 8.0, RRID:SCR_002798) as a contingency table (3 rows×2 columns). The distribution of categories within each cohort was visualized using a 100% stacked bar chart, in which each bar was normalized to the cohort total and displayed as the percentage of patients per category. Differences in the categorical distributions between responders and non-responders were assessed using a contingency table test (chi-square test; Fisher's exact test was used when expected cell counts were small, as appropriate). Statistical significance was defined as P<0.05, and non-significant results were annotated as “ns” on the figure.

### Results

Detection of TEX^{TF-Ag-α} PD-L1 using Exo-PROS assay: We have developed an Exo-PROS assay that enables the simultaneous detection of multiple TEX biomarkers on a single platform (29). The Exo-PROS assay employs surface plasmon resonance (SPR) as the sensing mechanism, allowing sensitive detection of different types of TEX biomarkers including proteins, microRNAs, and carbohydrate antigens. In this study, the Exo-PROS assay was used to first quantify the level of TEX TF-Ag-α and subsequently measure PD-L1 levels on these TF-Ag-α-expressing TEX (i.e., TEX^{TF-Ag-α}). Briefly, as shown in FIGS. 30A-30B, JAA-F11 antibodies specific to TF-Ag-α were immobilized on the surface of a gold-coated glass slide. Water and PBS were initially introduced to the biochip to collect background signals (I_{water} and I_{PBS}). Next, 10 μL of serum diluted in 40 μL of PBS was applied to the biochip to capture TEX^{TF-Ag-α} via the immobilized JAA-F11 antibodies. Binding of TEX^{TF-Ag-α} altered the local refractive index at the sensor surface, leading to changes in the intensity of reflected laser light and enabling optical detection of TEX^{TF-Ag-α} After unbound exosomes were removed by PBS washing, the intensity of reflected laser light (I_{TEX TF-Ag-α}) was recorded. The expression level of TEX TF-Ag-α is calculated using the Equation 1.

Next, to detect PD-L1 levels in TEX^{TF-Ag-α} fluorescent dye, APC labeled anti-PD-L1 antibodies (APC-PD-L1) were added and incubated on the biochip for 1 hour at RT. Excess APC-PD-L1 antibodies were removed by washing three times with PBS. The fluorescence signals from APC were measured by the avalanche photodetector (APD) positioned above the biochip. In Exo-PROS biosensor, the fluorescence signals are enhanced ~100 times by the compact SPR system, i.e. surface plasmon enhanced fluorescence spectroscopy (SPEFS). The fluorescence intensities recorded before and after the addition of APC-PD-L1 antibodies were used to calculate the expression level of TEX^{TF-Ag-α} PD-L1 according to the following equation.

TEX^{TF-Ag-α}PD-L1 Expression=(F_{after APC-PD-L1}−F_{before APC-PD-L1})/F_{before APC-PD-cL1}

Evaluation of TEX TF-Ag-α, TEX^{TF-Ag-α} PD-L1, TEX LG3BP, and TEX^{LG3BP} PD-L1 for predicting response to anti-PD-1 immunotherapy in a discovery cohort of NSCLC patients: In a discovery cohort of NSCLC patients (n=29), we compared the performance of TEX TF-Ag-α and TEX^{TF-Ag-α} PD-L1 in predicting response to anti-PD-1 treatment with that of TEX LG3BP and TEX^{LG3BP} PD-L1. LG3BP was selected for comparison because it has been reported as a promising circulating biomarker for distinguishing benign pulmonary nodules from malignant lesions (30-33). In our previous study, we further showed that exosomal LG3BP discriminated lung cancer patients from normal controls (including both low- and high-risk individuals), achieving an AUC of 0.96, with a sensitivity of 0.85 and a specificity of 0.97 (29).

The discovery cohort included 15 responders (complete response or partial response) and 14 non-responders (stable disease or progression). The clinical characteristics of these patients were summarized in TABLE 13 and TABLE 14 (No. 1-No. 29). The Exo-PROS assay was used to measure the levels of TEX TF-Ag-α, TEX^{TF-Ag-α} PD-L1, TEX LG3BP and TEX^{LG3BP} PD-L1 at both the pre-treatment baseline and at the end of first treatment cycle (i.e., 3-6 weeks post treatment). Our results demonstrated that TF-Ag-α serves as a superior biomarker for selectively capturing TEX and improving predictive performance.

TABLE 13
Characteristics of nonsmall cell lung cancer patients receiving
anti-PD-1 treatment.
n (%)

| | Discovery Set | | Validation Set | |
|---|---|---|---|---|
| Total patients | 29 | | 62 | |
| | Responder | Non-Responder | Responder | Non-Responder |
| | (n = 15) | (n = 14) | (n = 25) | (n = 37) |
| Gender | | | | |
| Female | 8 (53) | 7 (50) | 14 (56) | 23 (62) |
| Male | 7 (47) | 7 (50) | 11 (44) | 14 (38) |
| Age (years) | | | | |
| Mean | 66.4 | 68.6 | 66.7 | 69.4 |
| Median | 66 | 68 | 65 | 71 |
| Range | 58-79 | 57-84 | 39-88 | 50-89 |
| Stage | | | | |
| II | 0 (0) | 0 (0) | 0 (0) | 1 (3) |
| III | 4 (27) | 1 (7) | 7 (28) | 6 (16) |
| IV | 11 (73) | 13 (93) | 18 (72) | 30 (81) |

At the pre-treatment baseline, there were no significant differences in TEX TF-Ag-α or TEX^{TF-Ag-α} PD-L1 levels between responders (n=15) and non-responders (n=14) (FIGS. 31A-31B). Receiver operating characteristic (ROC) analysis demonstrated that each biomarker alone had limited predictive performance. TEX TF-Ag-α distinguished responders from non-responders with an AUC of 0.65, a sensitivity of 0.57, and a specificity of 0.80 (FIG. 31C). TEX^{TF-Ag-α} PD-L1 showed an AUC of 0.57, a sensitivity of 0.93, and a specificity of 0.27 (FIG. 31D). Combining the two biomarkers improved predictive performance, yielding an AUC of 0.77, a sensitivity of 0.64, and a specificity of 0.87 for distinguishing responders from non-responders (FIG. 32A).

At the end of the first treatment cycle, TEX TF-Ag-α levels were 0.64-fold lower in responders than in non-responders (1.74 vs. 2.72; p<0.05), whereas TEX^{TF-Ag-α} PD-L1 levels were 3.13-fold higher in responders compared with non-responders (0.098 vs. 0.031; p<0.001), effectively distinguishing the two groups (FIGS. 31E-31F). ROC analysis showed that each biomarker individually exhibited improved predictive performance relative to the pre-treatment baseline. TEX TF-Ag-α distinguished responders from non-responders with an AUC of 0.70, a sensitivity of 0.50, and a specificity of 0.93 (FIG. 31G). TEX^{TF-Ag-α} PD-L1 demonstrated an AUC of 0.90, a sensitivity of 0.86, and a specificity of 0.80 (FIG. 31H). Combining the two biomarkers yielded predictive performance comparable to TEX^{TF-Ag-α} PD-L1 alone, with an AUC of 0.88, a sensitivity of 0.86, and a specificity of 0.80 for distinguishing responders from non-responders (FIG. 32B).

Dynamic changes in biomarker levels were assessed by calculating fold changes between the end of the first treatment cycle and the pre-treatment baseline. No significant difference in TEX TF-Ag-α fold change was observed between responders and non-responders; the mean TEX TF-Ag-α fold change was 0.98 in responders and 1.23 in non-responders (FIGS. 31I-31J). In contrast, responders exhibited a significantly greater fold increase in TEX^{TF-Ag-α} PD-L1 levels compared with non-responders (p<0.01); the mean TEX^{TF-Ag-α} PD-L1 fold change was 3.49 in responders and 0.76 in non-responders (FIGS. 31K-31L). ROC analysis showed that fold-change-based metrics for each biomarker demonstrated improved predictive performance compared with pre-treatment baseline measurements, and comparable predictive performance to measurements obtained at the end of the first treatment cycle. TEX TF-Ag-α fold change distinguished responders from non-responders with an AUC of 0.65, a sensitivity of 0.57, and a specificity of 0.87 (FIG. 31M). TEX^{TF-Ag-α} PD-L1 fold change showed better predictive performance, with an AUC of 0.87, a sensitivity of 0.79, and a specificity of 0.87 (FIG. 31N). Combining the fold changes of the two biomarkers yielded slightly improved predictive performance compared with TEX^{TF-Ag-α} PD-L1 fold change alone, achieving an AUC of 0.88, a sensitivity of 0.86, and a specificity of 0.87 for distinguishing responders from non-responders (FIG. 32C).

In addition to TF-Ag-α, we used LG3BP to isolate TEXs and measured TEX LG3BP and TEX^{LG3BP} PD-L1 levels in the same serum samples using the Exo-PROS assay. Results were shown in FIGS. 33A-33N and FIGS. 34A-34C. At the pre-treatment baseline, no significant differences were observed in the TEX^{LG3BP} PD-L1 levels between responders and non-responders (FIG. 33B). Baseline TEX^{LG3BP} PD-L1 exhibited slightly better predictive performance than baseline TEX^{TF-Ag-α} PD-L1 (AUC: 0.62 vs. 0.57; sensitivity: 0.50 vs. 0.93; specificity: 0.87 vs. 0.27) (FIG. 33D). However, at the end of first treatment cycle, even though significantly higher levels of TEX^{LG3BP} PD-L1 were observed in responders than non-responders (0.082 vs. 0.037, p<0.05, FIG. 33F), end-of cycle 1 TEX^{LG3BP} PD-L1 showed inferior predictive performances compared with end-of-cycle 1 TEX^{TF-Ag-α} PD-L1 (AUC: 0.80 vs. 0.90; sensitivity: 0.79 vs. 0.86; specificity: 0.73 vs. 0.80) (FIG. 33H). When dynamic fold changes were assessed, no significant differences were observed in the TEX^{LG3BP} PD-L1 fold changes between responders and non-responders (FIGS. 33K-33L). The predictive performance of TEX^{LG3BP} PD-L1 fold change was also lower than that of TEX^{TF-Ag-α} PD-L1 fold change (AUC: 0.79 vs. 0.87; sensitivity: 0.64 vs. 0.79; specificity: 0.87 vs. 0.87) (FIG. 33N). These results demonstrated that TF-Ag-α is a superior biomarker for the selective capture of TEXs and for improving the predictive performance of PD-L1.

Evaluation of TEX TF-Ag-α and TEX^{TF-Ag-α} PD-L1 for predicting response to anti-PD-1 immunotherapy in an internal validation cohort of NSCLC patients: Encouraged by the findings from the discovery cohort, we next evaluated the predictive performance of TEX TF-Ag-α and TEX^{TF-Ag-α} PD-L1 in an internal validation cohort of patients with NSCLC (n=62). The internal validation cohort comprised 25 responders and 37 non-responders from the same institution, i.e., Roswell Park Comprehensive Cancer Center. The clinical characteristics of the patients were summarized in TABLE 13 and TABLE 14 (No. 30-No. 91).

At the pre-treatment baseline, no significant differences in TEX TF-Ag-α levels were observed between responders (n=25) and non-responders (n=37) (FIG. 35A). In contrast, responders showed significantly lower levels of TEX^{TF-Ag-α} PD-L1 compared with non-responders (p<0.01, FIG. 35B). ROC analysis indicated that TEX TF-Ag-α distinguished responders from non-responders with an AUC of 0.67, a sensitivity of 0.8, and a specificity of 0.57 at the cutoff of 1.80 (FIG. 35C). TEX^{TF-Ag-α} PD-L1 showed an AUC of 0.76, a sensitivity of 0.97, and a specificity of 0.56 at the cutoff of 0.018 (FIG. 35D). Combining the two biomarkers did not further improve predictive performance (FIG. 36A); the AUC, sensitivity, and specificity remained the same as for TEX^{TF-Ag-α} PD-L1 alone.

At the end of the first treatment cycle, TEX TF-Ag-α levels were comparable between responders and non-responders (FIG. 35E), whereas TEX^{TF-Ag-α} PD-L1 levels were 1.7-fold higher in responders compared with non-responders (p<0.05), effectively distinguishing the two groups (FIG. 35F). ROC analysis showed that TEX TF-Ag-α distinguished responders from non-responders with an AUC of 0.54, a sensitivity of 0.73, and a specificity of 0.48 at the cutoff of 2.13 (FIG. 35G), and TEX^{TF-Ag-α} PD-L1 demonstrated an AUC of 0.63, a sensitivity of 0.60, and a specificity of 0.68 at the cutoff of 0.036 (FIG. 35H). Combining the two biomarkers yielded a slightly better predictive performance for distinguishing responders from non-responders, with an AUC of 0.67, a sensitivity of 0.92, and a specificity of 0.38 at the cutoff of 0.28 (FIG. 36B). Compared with the discovery set, the predictive performance of TEX TF-Ag-α alone, TEX^{TF-Ag-α} PD-L1 alone, and their combination was all inferior, suggesting that biomarker levels measured at the end of first treatment cycle may not serve as reliable direct predictive markers.

Dynamic changes in biomarker levels were assessed by calculating fold changes between the end of the first treatment cycle and the pre-treatment baseline. No significant difference in TEX TF-Ag-α fold change was observed between responders and non-responders; the mean TEX TF-Ag-α fold change was 0.98 in responders and 1.19 in non-responders (FIGS. 35I-35J). In contrast, responders exhibited a significantly greater fold increase in TEX^{TF-Ag-α} PD-L1 levels compared with non-responders (p<0.0001); the mean TEX^{TF-Ag-α} PD-L1 fold change was 4.18 in responders and 0.82 in non-responders (FIGS. 35K-35L). ROC analysis showed TEX TF-Ag-α fold change distinguished responders from non-responders with an AUC of 0.59, a sensitivity of 0.64, and a specificity of 0.57 at the cutoff of 1.00 (FIG. 35M), TEX^{TF-Ag-α} PD-L1 fold change distinguished responders from non-responders with an AUC of 0.93, a sensitivity of 0.95, and a specificity of 0.8 at the cutoff of 1.59 (FIG. 35N), and the combination yielded slightly improved predictive performance, achieving an AUC of 0.94, a sensitivity of 0.97, and a specificity of 0.84 at the cutoff of 0.44 (FIG. 36C).

Compared with pre-treatment baseline measurements and those obtained at the end of the first treatment cycle, the TEX^{TF-Ag-α} PD-L1 fold change showed significantly enhanced predictive performance. Moreover, relative to the observations in the discovery set, the TEX^{TF-Ag-α} PD-L1 fold change also demonstrated improved predictive performance in the validation set. Collectively, these results indicate that the dynamic change of TEX^{TF-Ag-α} PD-L1 is a robust biomarker for predicting response to anti-PD-1 immunotherapy.

Predictive performance comparison among TEX^{TF-Ag-α} PD-L1, total exosomal PD-L1 and tissue PD-L1: To determine whether TEX^{TF-Ag-α} PD-L1 demonstrates superior predictive performance for response to anti-PD-1 immunotherapy, we compared it with total exosomal PD-L1 and tissue PD-L1 (FIGS. 37A-37I). FIGS. 38A-38P presented a side-by-side comparison of the predictive performance of TEX^{TF-Ag-α} PD-L1, total exosomal PD-L1 and tissue PD-L1. The analysis was conducted using the same patient cohort as the validation set, which included 25 responders and 37 non-responders. Their clinical characteristics were summarized in TABLE 14 (No. 30-No. 91).

At the pre-treatment baseline, TEX^{TF-Ag-α PD-L}1 levels were significantly lower in responders compared with non-responders (p<0.01, FIG. 35B), whereas total exosomal PD-L1 levels were comparable between the two groups (FIG. 37A). At the end of the first treatment cycle, both TEX^{TF-Ag-α} PD-L1 and total exosomal PD-L1 exhibited similar expression trends between responders and non-responders. TEX^{TF-Ag-α} PD-L1 expression was significantly higher in responders than non-responders (p<0.05; FIG. 35F), increasing by 4.18-fold in responders but decreasing by 0.82-fold in non-responders (FIGS. 35K-35L). Similarly, total exosomal PD-L1 levels were 1.5-fold higher in responders than non-responders (p<0.01; FIG. 37C), increasing by 1.38-fold in responders while decreasing by 0.8-fold in non-responders (FIGS. 37E-37F).

Compared with total exosomal PD-L1, TEX^{TF-Ag-α} PD-L1 demonstrated superior discriminative performance, especially when dynamic fold change was used as the biomarker. ROC analysis showed that at the pre-treatment baseline, TEX^{TF-Ag-α} PD-L1 distinguished responders from non-responders with an AUC of 0.76, a sensitivity of 0.97, and a specificity of 0.56 (FIG. 35D), outperforming total exosomal PD-L1, which achieved an AUC of 0.65, a sensitivity of 0.92, and a specificity of 0.44 (FIG. 37B). At the end of the first treatment cycle, TEX^{TF-Ag-α} PD-L1 achieved an AUC of 0.63, a sensitivity of 0.60, and a specificity of 0.68 (FIG. 35H), slightly inferior to total exosomal PD-L1 (AUC: 0.69; sensitivity: 0.92, specificity: 0.52) (FIG. 37D). When dynamic fold change was used as the biomarker, TEX^{TF-Ag-α} PD-L1 fold change yielded an AUC of 0.93, a sensitivity of 0.95, and a specificity of 0.80 (FIG. 35N), exceeding the performance of total exosomal PD-L1 (AUC: 0.89; sensitivity 0.81; specificity 0.88) (FIG. 37G). Collectively, these results demonstrate that selective capture of TEX enhances the predictive value of PD-L1 for response to anti-PD-1 immunotherapy.

Tissue PD-L1 tumor proportion scores (TPS) were compared between responders and non-responders. As shown in FIG. 37H, among the 25 responders, 17 had a PD-L1 TPS≥50%, 4 had a TPS between 1 and 49% and 4 had a TPS<1%. Among the 37 non-responders, 26 had a PD-L1 TPS≥50%, 8 had a TPS between 1 and 49% and 3 had a TPS<1%. ROC analysis indicated that tissue PD-L1 TPS demonstrated limited predictive performance, with an AUC of 0.51, sensitivity of 0.70, and specificity of 0.32 at the cutoff of 0.50 (FIG. 37I), substantially lower than that of TEX^{TF-Ag-α} PD-L1 fold change.

### Discussion

Tissue PD-L1 TPS remains the most widely used biomarker to guide anti-PD-1 therapy in NSCLC. However, tissue PD-L1 showed limited predictive value. In our cohort, tissue PD-L1 TPS showed an AUC of 0.51 in distinguishing responders from non-responders, consistent with previously reported modest accuracy. This limitation likely reflects spatial heterogeneity, temporal evolution of PD-L1 expression, and the static nature of a single pre-treatment biopsy.

In this study, we developed and validated a tumor-specific exosome-based liquid biopsy assay to predict response to anti-PD-1 immunotherapy in patients with NSCLC. By leveraging the tumor-specific glycoantigen TF-Ag-α to selectively capture TEXs, we demonstrated that dynamic changes in TEX^{TF-Ag-α} PD-L1 provide robust and reproducible predictive performance. Across independent discovery and validation cohorts, the fold change of TEX^{TF-Ag-α} PD-L1 after the first treatment cycle consistently distinguished responders from non-responders with high accuracy, achieving an AUC of 0.93, a sensitivity of 0.95, and a specificity of 0.80 at the cutoff of 1.59. When the fold change of TEX^{TF-Ag-α} PD-L1 was combined with TEX TF-Ag-α fold change, the predictive performance was further improved to an AUC of 0.94, sensitivity of 0.97, and specificity of 0.84. Importantly, this performance exceeded that of total exosomal PD-L1, underscoring the value of TF-Ag-α-positive, tumor-specific exosome isolation for improving biomarker precision.

A major challenge in using exosomal PD-L1 as a biomarker is that PD-L1 is expressed not only by tumor cells but also by multiple immune cell populations. As a result, total exosomal PD-L1 measurements may reflect a mixture of tumor- and immune-derived vesicles, potentially confounding interpretation. Our results directly address this limitation. Compared with total exosomal PD-L1, TEX^{TF-Ag-α} PD-L1 consistently showed superior predictive performance at baseline and, more notably, when fold change was used as the metric. These findings highlight the importance of selectively enriching TEXs prior to biomarker quantification. TF-Ag-α, a tumor-specific glycoepitope exposed due to aberrant glycosylation in cancer cells, enables precise capture of TEXs while minimizing background noise from non-malignant sources. The comparison with LG3BP-based capture further supports this conclusion, as TF-Ag-α-based isolation outperformed LG3BP in predicting treatment response. Collectively, these results indicate that tumor specificity at the exosome isolation step is critical for maximizing the clinical utility of circulating PD-L1 measurements.

Another key finding of this study is that dynamic changes in TEX^{TF-Ag-α} PD-L1, rather than absolute levels measured at the pre-treatment baseline or at the end of the first treatment cycle, provide the strongest predictive signal. In both the discovery and validation cohorts, the fold change in TEX^{TF-Ag-α} PD-L1 between baseline and the end of the first treatment cycle consistently demonstrated high AUC, sensitivity, and specificity for predicting response to anti-PD-1 therapy. Interestingly, responders exhibited a significant increase (~4.18-fold) in TEX^{TF-Ag-α} PD-L1 levels after initiation of anti-PD-1 therapy, whereas non-responders showed a moderate decrease (~20%). This observation agrees with results reported by other groups and may reflect an adaptive tumor response to immune activation. Effective PD-1 blockade can reinvigorate tumor-specific T cells, leading to enhanced immune pressure within the tumor microenvironment. In turn, tumor cells may upregulate PD-L1 expression and release PD-L1-bearing exosomes as a compensatory immune evasion mechanism. Thus, an early rise in tumor-specific exosomal PD-L1 may serve as a pharmacodynamic marker of immune engagement and therapeutic activity. In contrast, absence of such a dynamic response may indicate poor response or resistance to therapy. These findings align with the concept that early on-treatment biomarkers can outperform static pre-treatment measurements in immunotherapy, particularly given the complex and evolving interactions between tumor and immune system.

Importantly, serum-based monitoring circumvents limitations of tissue biopsy and avoids the interpretive challenges of imaging, such as pseudo-progression and delayed radiographic responses. The Exo-PROS biosensor platform used in this study allows simultaneous quantification of TEX TF-Ag-α and TEX^{TF-Ag-α} PD-L1 from small serum volumes, supporting feasibility for routine clinical implementation. As a minimally invasive assay, this approach may be particularly valuable for patients with advanced or inoperable NSCLC, where repeated tissue biopsies are impractical.

Several limitations should be acknowledged. First, although the study included independent discovery and validation cohorts, the overall sample size remains moderate. Larger, multi-center prospective studies are needed to validate these findings. Second, we only evaluated samples collected at the early stage of treatment. The performance of TEX^{TF-Ag-α} PD-L1 in predicting and monitoring long-term treatment responses remains to be determined and should be investigated using longitudinal samples collected at later time points. Third, TEX TF-Ag-α and TEX LG3BP did not demonstrate strong predictive performance in this study, which may be because samples were collected at an early stage of treatment, when tumor size may not yet have decreased sufficiently to reflect changes in these two tumor-associated biomarkers. Their efficacy in predicting and monitoring responses to ICIs should be further investigated using samples collected at later time points. Fourth, NSCLC patients in this study received PD-1 inhibitors (pembrolizumab, nivolumab, and cemiplimab), and the predictive performance of TEX^{TF-Ag-α} PD-L1 was therefore assessed only in the context of PD-1 blockade. Future studies should evaluate its predictive value in patients treated with other immune checkpoint inhibitors, such as PD-L1 inhibitors, as treatment differences may influence exosomal dynamics. Finally, future investigations should also explore integration of TEX^{TF-Ag-α} PD-L1 dynamics with other circulating biomarkers, such as circulating tumor DNA or immune cell profiling, to develop multi-parametric predictive models.

### Conclusion

In summary, we have demonstrated that selective capture of TEXs using TF-Ag-α substantially enhances the predictive value of exosomal PD-L1 for anti-PD-1 immunotherapy in NSCLC. Dynamic fold changes in TEX^{TF-Ag-α} PD-L1 after the first treatment cycle provide a highly accurate, minimally invasive biomarker for early response assessment, outperforming both total exosomal PD-L1 and tissue PD-L1 TPS. The high sensitivity and specificity achieved by TEX^{TF-Ag-α} PD-L1 fold change indicate strong potential for early response stratification. Patients unlikely to benefit from anti-PD-1 therapy could be identified within weeks of treatment initiation, enabling earlier therapeutic adjustments and minimizing unnecessary exposure to immune-related adverse events. Conversely, patients demonstrating favorable biomarker dynamics could continue therapy with increased confidence, even in cases where imaging findings are ambiguous. The superior predictive performance of TEX^{TF-Ag-α} PD-L1 observed here suggests that tumor-specific exosomal PD-L1 may complement or potentially outperform tissue-based assays in predicting and monitoring response to anti-PD-1 therapy. These findings warrant further validation in larger, prospective clinical trials.

### REFERENCES

Abdullah P, et al. Synthetic 2D mammography versus standard 2D digital mammography: a diagnostic test accuracy systematic review and meta-analysis. American Journal of Roentgenology 2021; 217(2):314-25.
Almogren A, et al. Anti-Thomsen-Friedenreich-Ag (anti-TF-Ag) potential for cancer therapy. Frontiers in Bioscience-Scholar 2012; 4(3):840-63.
Chen G, et al. Exosomal PD-L1 contributes to immunosuppression and is associated with anti-PD-1 response. Nature. 2018, 560, 382-386.
Cui Q, et al. Prognostic significance of blood-based PD-L1 analysis in patients with non-small cell lung cancer undergoing immune checkpoint inhibitor therapy: a systematic review and meta-analysis. World J Surg Oncol. 2023, 21(1), 318.
Davis A A, Patel V G. The role of PD-L1 expression as a predictive biomarker: an analysis of all US Food and Drug Administration (FDA) approvals of immune checkpoint inhibitors. J ImmunoTher Cancer. 2019, 7, 278.
Doroshow D B, et al. PD-L1 as a biomarker of response to immune-checkpoint inhibitors. Nat Rev Clin Oncol. 2021, 18(6), 345-362.
Friedenreich V. The Thomsen hemagglutination phenomenon: Production of a specific receptor quality in red corpuscles by bacterial activity. Levin & Munksgaard; 1930.
Ghazal D, et al. Therapeutic efficacy of the humanized JAA-F11 anti-Thomsen-Friedenreich antibody constructs H2aL2a and H3L3 in human breast and lung cancer xenograft models. Oncotarget 2022; 13:1155.
Glinskii O V, et al. Endothelial integrin α3β1 stabilizes carbohydrate-mediated tumor/endothelial cell adhesion and induces macromolecular signaling complex formation at the endothelial cell membrane. Oncotarget 2014; 5(5):1382-1389.
Gopal M, et al. Screening for lung cancer with low-dose computed tomography: a systematic review and meta-analysis of the baseline findings of randomized controlled trials. Journal of thoracic oncology 2010; 5(8):1233-9.
Gunasekaran M, et al. Exosomal PD-L1 expression as non-invasive biomarker for immune checkpoint inhibitors in non-small cell lung cancer. J Immunol. 2020, 204 (1 Supplement) 90.10.
Hanash S M, et al. Blood based biomarkers beyond genomics for lung cancer screening. Translational lung cancer research 2018; 7(3):327.
Hanisch F, Baldus S. The Thomsen-Friedenreich (TF) antigen: a critical review on the structural, biosynthetic and histochemica aspects of a pancarcinoma-associated antigen. Histology and histopathology 1997.
Heimburg J, et al. Inhibition of spontaneous breast cancer metastasis by anti-Thomsen-Friedenreich antigen monoclonal antibody JAA-F11. Neoplasia 2006; 8(11):939-48.
Hsu C-C, et al. Simultaneous Detection of Tumor Derived Exosomal Protein-MicroRNA Pairs with an Exo-PROS Biosensor for Cancer Diagnosis. ACS nano 2023; 17(9):8108-22.
Hsu M-T, et al. Exosomal proteins and lipids as potential biomarkers for lung cancer diagnosis, prognosis, and treatment. Cancers 2022; 14(3):732.
Huang H, et al. The potential diagnostic value of extracellular vesicle miRNA for human non-small cell lung cancer: A systematic review and meta-analysis. Expert Review of Molecular Diagnostics 2021; 21(8):823-36.
Ignatiadis M, et al. Liquid biopsy enters the clinic-implementation issues and future challenges. Nature reviews Clinical oncology 2021; 18(5):297-312.
Im H, et al. Nano-plasmonic exosome diagnostics. Expert review of molecular diagnostics 2015; 15(6):725-33.
Jahr S, et al. DNA fragments in the blood plasma of cancer patients: quantitations and evidence for their origin from apoptotic and necrotic cells. Cancer research 2001; 61(4):1659-65.
Kabe Y, et al. Development of a highly sensitive device for counting the number of disease-specific exosomes in human sera. Clinical chemistry 2018; 64(10):1463-73.
Karacosta L G, et al. Preclinical Analysis of JAA-F11, a Specific Anti-Thomsen-Friedenreich Antibody via Immunohistochemistry and In vivo Imaging. Translational oncology 2018; 11(2):450-66.
Karacosta L G, et al. Preclinical Analysis of JAA-F11, a Specific Anti-Thomsen-Friedenreich Antibody via Immunohistochemistry and In vivo Imaging. Transl Oncol. 2018, 11(2), 450-466.
Lee S J, et al. Exosomal Del-1 as a potent diagnostic marker for breast cancer: prospective cohort study. Clinical Breast Cancer 2021; 21(6):e748-e56.
Lehman C D, et al. Diagnostic Accuracy of Digital Screening Mammography With and Without Computer Aided Detection. JAMA Internal Medicine 2015; 175(11):1828-37 doi 10.1001/jamainternmed.2015.5231.
Li X J, et al. A blood-based proteomic classifier for the molecular characterization of pulmonary nodules. Sci Transl Med. 2013, 5(207), 207ra142.
Li Y, et al. Molecular identification of tumor-derived extracellular vesicles using thermophoresis-mediated DNA computation. Journal of the American Chemical Society 2021; 143(3):1290-5.
Liu C, et al. Non-invasive detection of exosomal MicroRNAs via tethered cationic lipoplex nanoparticles (tCLN) biochip for lung cancer early detection. Frontiers in Genetics 2020; 11:258.
Liu C, et al. Sensitive detection of exosomal proteins via a compact surface plasmon resonance biosensor for cancer diagnosis. ACS sensors 2018; 3(8):1471-9.
Meckes Jr D G, Raab-Traub N. Microvesicles and viral infection. Journal of virology 2011; 85(24):12844-54.
Monzavi-Karbassi B, et al. Tumor-associated glycans and immune surveillance. Vaccines 2013; 1(2):174-203.
Moon P-G, et al. Fibronectin on circulating extracellular vesicles as a liquid biopsy to detect breast cancer. Oncotarget 2016; 7(26):40189.
Moon P-G, et al. Identification of developmental endothelial locus-1 on circulating extracellular vesicles as a novel biomarker for early breast cancer detection. Clinical cancer research 2016; 22(7):1757-66.
Mun B, et al. An immuno-magnetophoresis-based microfluidic chip to isolate and detect HER2-Positive cancer-derived exosomes via multiple separation. Biosensors and Bioelectronics 2023:115592.
Neurohr K J, et al. Binding of disaccharides by peanut agglutinin as studied by ultraviolet difference spectroscopy. European journal of biochemistry 1982; 123(2):305-10.
Normanno N, et al. Guide to detecting epidermal growth factor receptor (EGFR) mutations in ctDNA of patients with advanced non-small-cell lung cancer. Oncotarget 2017; 8(7):12501.
Peled N, Ilouze M. Screening for lung cancer: what comes next? Volume 33: American Society of Clinical Oncology; 2015. p 3847-8.
Pisano E D, et al. Diagnostic Accuracy of Digital versus Film Mammography: Exploratory Analysis of Selected Population Subgroups in DMIST. Radiology 2008; 246(2):376-83 doi 10.1148/radio1.2461070200.
Ribli D, et al. Detecting and classifying lesions in mammograms with Deep Learning. Scientific Reports 2018; 8(1):4165 doi 10.1038/s41598-018-22437-z.
Rittenhouse-Diakun K, et al. Development and characterization of monoclonal antibody to T-Antigen:(Galβ1-3GalNAc-α-O). Hybridoma 1998; 17(2):165-73.
Shao J, et al. Deep learning empowers lung cancer screening based on mobile low-dose computed tomography in resource-constrained sites. Frontiers in Bioscience-Landmark 2022; 27(7):212.
Shimada Y, et al. Serum-derived exosomal PD-L1 expression to predict anti-PD-1 response and in patients with non-small cell lung cancer. Sci Rep. 2021, 11(1), 7830.
Shrivastava A, Gupta V B. Methods for the determination of limit of detection and limit of quantitation of the analytical methods. Chron Young Sci 2011; 2(1):21-5.
Silvestri G A, et al; PANOPTIC Trial Team. Assessment of Plasma Proteomics Biomarker's Ability to Distinguish Benign From Malignant Lung Nodules: Results of the PANOPTIC (Pulmonary Nodule Plasma Proteomic Classifier) Trial. Chest. 2018, 154(3), 491-500.
Siravegna G, et al. Integrating liquid biopsies into the management of cancer. Nature reviews Clinical oncology 2017; 14(9):531-48.
Springer G F. T and Tn, general carcinoma autoantigens. Science 1984; 224(4654):1198-206.
Takanami I. Expression of Thomsen-Friedenreich antigen as a marker of poor prognosis in pulmonary adenocarcinoma. Oncol Rep 1999; 6:341-4
Tanner N T, et al. Assessment of Integrated Classifier's Ability to Distinguish Benign From Malignant Lung Nodules: Extended Analyses and 2-Year Follow-Up Results of the PANOPTIC (Pulmonary Nodule Plasma Proteomic Classifier) Trial. Chest. 2021, 159(3), 1283-1287.
Tati S, et al. Humanization of JAA-F11, a highly specific anti-thomsen-friedenreich pancarcinoma antibody and in vitro efficacy analysis. Neoplasia 2017; 19(9):716-33.
Team NLSTR. Reduced lung-cancer mortality with low-dose computed tomographic screening. New England Journal of Medicine 2011; 365(5):395-409.
Thomsen O. Ein vermehrungsfahiges Agens als Veranderer des isooagglutinotorischen Verhaltens der roten Blutkarperchen, eine bisher unbekannte Quelle der Fehlbestimmung. 1927.
Vachani A, et al. Validation of a multiprotein plasma classifier to identify benign lung nodules. J Thorac Oncol. 2015, 10(4), 629-37.
Wang M, et al. Diagnostic and predictive value of liquid biopsy-derived exosome miR-21 for breast cancer: a systematic review and meta-analysis. Expert Review of Molecular Diagnostics 2023; 23(4):315-24.
Yang Q, et al. Novel Biomarkers of Dynamic Blood PD-L1 Expression for Immune Checkpoint Inhibitors in Advanced Non-Small-Cell Lung Cancer Patients. Front Immunol. 2021, 12, 665133.
Yang Y, et al. An immuno-biochip selectively captures tumor-derived exosomes and detects exosomal RNAs for cancer diagnosis. ACS applied materials & interfaces 2018; 10(50):43375-86.
Yin L, et al. The role of exosomes in lung cancer metastasis and clinical applications: an updated review. Journal of Translational Medicine 2021; 19:1-16.
Yu L-G. The oncofetal Thomsen-Friedenreich carbohydrate antigen in cancer progression. Glycoconjugate journal 2007; 24(8):411-20.
Zhang H, et al. Value of miR-21 levels as potential biomarkers in the early diagnosis of hepatocellular carcinoma: a meta-analysis. Biomarkers 2021; 26(7):586-97.

### TABLES

TABLE 5
Characteristics of Lung Cancer Patients and Controls.

| | | | | | | |
|---|---|---|---|---|---|---|
| | | | | | | Exosomal |
| | | | | | | TF-Ag-α |
| | | | Gen- | | | level |
| ID | Cancer type | Race | der | Age | Stage | (A.U.) |

Training set (41 normal controls and 40 cancer cases)

| | | | | | | |
|---|---|---|---|---|---|---|
| 1 | Normal control (low risk) | White | M | 50 | | 0.35 |
| 2 | Normal control (low risk) | White | F | 65 | | 0.28 |
| 3 | Normal control (low risk) | White | F | 56 | | 0.04 |
| 4 | Normal control (low risk) | White | M | 57 | | 0.20 |
| 5 | Normal control (low risk) | White | F | 60 | | −0.23 |
| 6 | Normal control (low risk) | White | M | 61 | | 0.22 |
| 7 | Normal control (low risk) | White | M | 60 | | −0.64 |
| 8 | Normal control (low risk) | White | M | 68 | | 0.53 |
| 9 | Normal control (low risk) | White | M | 65 | | −0.26 |
| 10 | Normal control (low risk) | Black | M | 50 | | −0.34 |
| 11 | Normal control (low risk) | White | F | 77 | | 0.02 |
| 12 | Normal control (low risk) | White | M | 54 | | 0.08 |
| 13 | Normal control (low risk) | White | F | 70 | | 0.15 |
| 14 | Normal control (low risk) | White | F | 76 | | 0.10 |
| 15 | Normal control (low risk) | White | F | 60 | | 0.16 |
| 16 | Normal control (low risk) | White | F | 64 | | 0.43 |
| 17 | Normal control (low risk) | White | F | 56 | | −0.14 |
| 18 | Normal control (low risk) | White | M | 49 | | −0.04 |
| 19 | Normal control (low risk) | White | M | 73 | | −0.34 |
| 20 | Normal control (low risk) | White | M | 57 | | 0.57 |
| 21 | Normal control (high risk) | White | M | 53 | | 1.41 |
| 22 | Normal control (high risk) | White | M | 42 | | 1.23 |
| 23 | Normal control (high risk) | White | M | 55 | | 0.12 |
| 24 | Normal control (high risk) | White | F | 55 | | 0.61 |
| 25 | Normal control (high risk) | White | F | 55 | | 0.22 |
| 26 | Normal control (high risk) | White | M | 73 | | −0.08 |
| 27 | Normal control (high risk) | White | M | 52 | | 0.59 |
| 28 | Normal control (high risk) | White | F | 48 | | −0.54 |
| 29 | Normal control (high risk) | White | M | 62 | | 0.08 |
| 30 | Normal control (high risk) | White | F | 71 | | 0.23 |
| 31 | Normal control (high risk) | White | F | 63 | | 0.29 |
| 32 | Normal control (high risk) | White | M | 55 | | −0.33 |
| 33 | Normal control (high risk) | Un- | M | 58 | | 0.20 |
| | | known | | | | |
| 34 | Normal control (high risk) | White | M | 57 | | 0.34 |
| 35 | Normal control (high risk) | White | F | 72 | | −0.19 |
| 36 | Normal control (high risk) | White | M | 69 | | 0.39 |
| 37 | Normal control (high risk) | White | M | 53 | | −0.11 |
| 38 | Normal control (high risk) | White | F | 51 | | −0.11 |
| 39 | Normal control (high risk) | White | F | 52 | | 0.07 |
| 40 | Normal control (high risk) | White | M | 65 | | −0.04 |
| 41 | Normal control (high risk) | White | M | 66 | | −0.01 |
| 42 | Squamous cell carcinoma, | White | F | 52 | 1B | 0.64 |
| | keratinizing, NOS | | | | | |
| 43 | Squamous cell carcinoma, | White | M | 59 | 1A | 0.66 |
| | NOS | | | | | |
| 44 | Adenocarcinoma, NOS | White | F | 75 | 1A | 0.79 |
| 45 | Adenocarcinoma, NOS | White | F | 56 | 1A | 0.53 |
| 46 | Adenocarcinoma, NOS | White | M | 60 | 1A | 1.99 |
| 47 | Squamous cell carcinoma, | White | M | 76 | 1A | 1.21 |
| | NOS | | | | | |
| 48 | Adenocarcinoma, NOS | White | M | 60 | 1B | 0.89 |
| 49 | Adenocarcinoma, NOS | White | M | 53 | 1B | 2.97 |
| 50 | Small cell lung cancer | White | F | 78 | 1A2 | 0.71 |
| 51 | Small cell lung cancer | White | F | 73 | 1A3 | 0.51 |
| 52 | Adenocarcinoma with | White | F | 74 | 2 | 0.66 |
| | mixed subtypes | | | | | |
| 53 | Squamous cell carcinoma, | White | M | 66 | 2A | 0.65 |
| | NOS | | | | | |
| 54 | Adenocarcinoma, NOS | White | F | 70 | 2B | 0.67 |
| 55 | Adenocarcinoma, NOS | White | F | 61 | 2B | 0.45 |
| 56 | Adenocarcinoma, NOS | White | M | 57 | 2B | 0.76 |
| 57 | Squamous cell carcinoma, | White | M | 65 | 2B | 1.11 |
| | NOS | | | | | |
| 58 | Adenocarcinoma, NOS | White | M | 63 | 2B | 1.29 |
| 59 | Small cell lung cancer | White | F | 70 | 2B | 2.31 |
| 60 | Mucinous adenocarcinoma | White | M | 57 | 3B | 0.87 |
| 61 | Squamous cell carcinoma, | White | M | 54 | 3A | 0.32 |
| | NOS | | | | | |
| 62 | Adenocarcinoma, NOS | White | F | 58 | 3B | 4.10 |
| 63 | Adenocarcinoma, NOS | White | F | 64 | 3A | 0.72 |
| 64 | Adenocarcinoma, NOS | White | F | 55 | 3B | 1.11 |
| 65 | Squamous cell carcinoma, | White | F | 63 | 3A | 2.06 |
| | NOS | | | | | |
| 66 | Squamous cell carcinoma, | White | M | 67 | 3B | 1.01 |
| | NOS | | | | | |
| 67 | Adenocarcinoma, NOS | White | F | 47 | 3A | 0.90 |
| 68 | Adenocarcinoma, NOS | White | F | 77 | 3A | 0.44 |
| 69 | Small cell lung cancer | White | F | 71 | 3B | 2.49 |
| 70 | Small cell lung cancer | White | F | 60 | 3A | 0.81 |
| 71 | Small cell lung cancer | White | M | 61 | 3B | 0.74 |
| 72 | Small cell lung cancer | White | M | 62 | 3A | 0.57 |
| 73 | Small cell lung cancer | White | M | 62 | 3A | 1.10 |
| 74 | Adenocarcinoma, NOS | White | F | 73 | 4 | 0.87 |
| 75 | Adenocarcinoma, NOS | White | F | 69 | 4 | 2.51 |
| 76 | Squamous cell carcinoma, | White | M | 58 | 4 | 1.55 |
| | NOS | | | | | |
| 77 | Adenocarcinoma, NOS | White | M | 62 | 4A | 0.61 |
| 78 | Squamous cell carcinoma, | White | M | 77 | 4B | 0.88 |
| | NOS | | | | | |
| 79 | Squamous cell carcinoma, | White | F | 70 | 4B | 0.73 |
| | NOS | | | | | |
| 80 | Small cell lung cancer | Black | M | 70 | 4A | 0.93 |
| 81 | Small cell lung cancer | White | M | 68 | 4B | 1.11 |

Test set (6 normal controls and 20 cancer cases)

| | | | | | | |
|---|---|---|---|---|---|---|
| 82 | Normal control | White | M | 61 | | −1.26 |
| 83 | Normal control | White | M | 65 | | −0.72 |
| 84 | Normal control | White | M | 63 | | −0.45 |
| 85 | Normal control* | White | F | 77 | | −1.09 |
| 86 | Normal control* | White | F | 62 | | −0.47 |
| 87 | Normal control* | White | F | 48 | | −0.81 |
| 88 | Adenocarcinoma, NOS | White | M | 69 | 1 | 0.70 |
| 89 | Squamous cell carcinoma, | White | M | 57 | 1 | 0.48 |
| | NOS | | | | | |
| 90 | Adenocarcinoma, NOS | White | F | 52 | 1 | 0.49 |
| 91 | Adenocarcinoma, NOS | White | M | 64 | 1 | 0.88 |
| 92 | Adenocarcinoma, NOS | White | F | 70 | 1 | 0.49 |
| 93 | Adenocarcinoma, NOS | White | M | 68 | 2 | 0.72 |
| 94 | Adenocarcinoma, NOS | White | M | 48 | 2 | 0.76 |
| 95 | Adenocarcinoma with | White | M | 61 | 2 | 0.76 |
| | mixed subtypes | | | | | |
| 96 | Adenocarcinoma with | White | F | 64 | 2 | 0.45 |
| | mixed subtypes | | | | | |
| 97 | Squamous cell carcinoma, | White | M | 49 | 2 | 0.71 |
| | NOS | | | | | |
| 98 | Adenocarcinoma, NOS | White | F | 62 | 3 | 0.48 |
| 99 | Adenocarcinoma, NOS | White | F | 55 | 3B | 1.39 |
| 100 | Small cell carcinoma, NOS | White | M | 62 | 3 | 1.03 |
| 101 | Small cell carcinoma, NOS | White | F | 62 | 3 | 0.58 |
| 102 | Small cell carcinoma, NOS | White | F | 51 | 3 | 0.70 |
| 103 | Adenocarcinoma, NOS | White | F | 41 | 4 | −0.15 |
| 104 | Adenocarcinoma, NOS | White | M | 55 | 4 | 0.51 |
| 105 | Small cell carcinoma, NOS | White | F | 67 | 4 | 0.42 |
| 106 | Small cell carcinoma, NOS | White | M | 66 | 4 | 0.61 |
| 107 | Small cell carcinoma, NOS | White | F | 64 | 4 | 0.66 |

*These normal controls are the same normal controls #100, #101 and #102 in TABLE 6.

TABLE 6
Characteristics of Breast Cancer Patients and Controls.

| | | | | | | |
|---|---|---|---|---|---|---|
| | | | | | | Exo- |
| | | | | | | somal |
| | | | | | | TF-Ag- |
| | | | Gen- | | | α level |
| ID | Cancer type | Race | der | Age | Stage | (A.U.) |

Training set (29 normal controls and 70 cancer cases)

| | | | | | | |
|---|---|---|---|---|---|---|
| 1 | Normal control | White | F | 36 | | 0.01 |
| 2 | Normal control | White | F | 44 | | 0.30 |
| 3 | Normal control | White | F | 48 | | 0.46 |
| 4 | Normal control | White | F | 55 | | 0.30 |
| 5 | Normal control | White | F | 57 | | 0.09 |
| 6 | Normal control | White | F | 69 | | 0.04 |
| 7 | Normal control | White | F | 65 | | −0.06 |
| 8 | Normal control | White | F | 74 | | 0.40 |
| 9 | Normal control | White | F | 73 | | −0.29 |
| 10 | Normal control | White | F | 50 | | −0.52 |
| 11 | Normal control | White | F | 52 | | −0.42 |
| 12 | Normal control | White | F | 63 | | 0.28 |
| 13 | Normal control | White | F | 56 | | 0.22 |
| 14 | Normal control | White | F | 64 | | 0.15 |
| 15 | Abnormal mammogram | Black | F | 41 | Benign | 0.08 |
| 16 | Atypical ductal hyperplasia | White | F | 77 | Benign | 0.18 |
| | (ADH) | | | | | |
| 17 | Atypical ductal hyperplasia | Black | F | 47 | Benign | 0.66 |
| 18 | Atypical ductal hyperplasia | White | F | 59 | Benign | −0.08 |
| 19 | Atypical lobular | White | F | 41 | Benign | −0.21 |
| | hyperplasia (ALH) | | | | | |
| 20 | Atypical lobular | White | F | 52 | Benign | 0.10 |
| | hyperplasia | | | | | |
| 21 | Atypical lobular | White | F | 55 | Benign | −0.31 |
| | hyperplasia | | | | | |
| 22 | Fibroadenoma | White | F | 47 | Benign | −0.53 |
| 23 | Fibroadenoma | White | F | 50 | Benign | −0.20 |
| 24 | Fibroadenoma | White | F | 57 | Benign | −0.35 |
| 25 | Fibrocystic Change | Black | F | 53 | Benign | −0.86 |
| 26 | Fibrocystic Change | White | F | 56 | Benign | −0.83 |
| 27 | Intraductal Papilloma | White | F | 49 | Benign | −0.08 |
| 28 | Intraductal Papilloma | White | F | 55 | Benign | 0.22 |
| 29 | Intraductal Papilloma | White | F | 64 | Benign | 0.47 |
| 30 | Ductal carcinoma in situ | White | F | 64 | 0 | 1.34 |
| | (DCIS) | | | | | |
| 31 | DCIS | White | F | 56 | 0 | 1.92 |
| 32 | DCIS | White | F | 65 | 0 | 0.21 |
| 33 | DCIS | White | F | 33 | 0 | 1.85 |
| 34 | DCIS | White | F | 70 | 0 | 3.04 |
| 35 | DCIS | Asian | F | 61 | 0 | 0.49 |
| 36 | DCIS | White | F | 44 | 0 | 1.51 |
| 37 | DCIS | White | F | 62 | 0 | 1.42 |
| 38 | DCIS | White | F | 76 | 0 | 2.04 |
| 39 | Invasive carcinoma, no | un- | F | 61 | 0 | 0.97 |
| | special type | known | | | | |
| 40 | Invasive carcinoma, no | White | F | 54 | 0 | 2.57 |
| | special type | | | | | |
| 41 | Intraductal carcinoma, | White | F | 48 | 0 | 2.19 |
| | noninfiltrating | | | | | |
| 42 | Infiltrating duct carcinoma | White | F | 48 | 1 | 3.20 |
| 43 | Infiltrating duct carcinoma | White | F | 58 | 1 | 0.69 |
| 44 | Infiltrating duct carcinoma | White | F | 57 | 1 | 0.71 |
| 45 | Infiltrating duct carcinoma | White | F | 68 | 1 | 0.66 |
| 46 | Infiltrating duct carcinoma | White | F | 65 | 1 | 0.48 |
| 47 | Infiltrating duct carcinoma | White | F | 77 | 1 | 1.11 |
| 48 | Infiltrating duct carcinoma | White | F | 61 | 1 | 0.54 |
| 49 | Infiltrating duct carcinoma | White | F | 69 | 1 | 2.26 |
| 50 | Invasive carcinoma | White | F | 48 | 1 | 0.77 |
| 51 | Infiltrating duct carcinoma | White | F | 75 | 1 | 0.94 |
| 52 | Infiltrating duct carcinoma | White | F | 64 | 1B | 1.52 |
| 53 | Infiltrating duct carcinoma | Black | F | 59 | 1A | 1.02 |
| 54 | Infiltrating duct carcinoma | White | F | 56 | 1 | 0.64 |
| 55 | Infiltrating duct carcinoma | White | F | 51 | 1 | 0.23 |
| 56 | Infiltrating duct carcinoma | White | F | 38 | 2 | 1.65 |
| 57 | Infiltrating duct carcinoma | White | F | 69 | 2 | 1.53 |
| 58 | Infiltrating duct carcinoma | Black | F | 41 | 2 | 0.71 |
| 59 | Infiltrating duct carcinoma | White | F | 39 | 2 | 0.76 |
| 60 | Infiltrating duct carcinoma | White | F | 42 | 2 | 0.41 |
| 61 | Infiltrating duct carcinoma | White | F | 73 | 2 | 0.32 |
| 62 | Invasive carcinoma, no | White | F | 75 | 2A | 3.08 |
| | special type | | | | | |
| 63 | Invasive carcinoma, no | White | F | 80 | 2A | 1.14 |
| | special type | | | | | |
| 64 | Invasive carcinoma, no | White | F | 64 | 2A | 2.11 |
| | special type | | | | | |
| 65 | Invasive carcinoma, no | White | F | 72 | 2A | 1.40 |
| | special type | | | | | |
| 66 | Invasive carcinoma, no | White | F | 57 | 2B | 4.23 |
| | special type | | | | | |
| 67 | Invasive carcinoma, no | White | F | 54 | 2B | 1.96 |
| | special type | | | | | |
| 68 | Infiltrating duct carcinoma | White | F | 34 | 2B | 1.38 |
| 69 | Infiltrating duct carcinoma | White | F | 44 | 2A | 1.38 |
| 70 | Infiltrating duct carcinoma | White | F | 49 | 2A | 1.10 |
| 71 | Infiltrating duct and lobular | White | F | 51 | 3A | 1.15 |
| | carcinoma | | | | | |
| 72 | Invasive carcinoma, no | White | F | 62 | 3A | 0.86 |
| | special type | | | | | |
| 73 | Infiltrating duct carcinoma | White | F | 62 | 3A | 3.02 |
| 74 | Infiltrating duct carcinoma | White | F | 30 | 3B | 1.10 |
| 75 | Infiltrating duct carcinoma | White | F | 79 | 3 | 1.43 |
| 76 | Infiltrating duct carcinoma | White | F | 46 | 3 | 1.64 |
| 77 | Infiltrating duct carcinoma | White | F | 48 | 3 | 0.58 |
| 78 | Infiltrating duct carcinoma | White | F | 62 | 3 | 0.82 |
| 79 | Infiltrating duct carcinoma | Black | F | 46 | 3 | 1.45 |
| 80 | Infiltrating duct carcinoma | White | F | 62 | 3 | 1.11 |
| 81 | Infiltrating duct carcinoma | White | F | 57 | 3 | 0.86 |
| 82 | Infiltrating duct carcinoma | Asian | F | 37 | 3 | 0.93 |
| 83 | Infiltrating duct carcinoma | White | F | 56 | 3 | 1.89 |
| 84 | Infiltrating duct and lobular | White | F | 51 | 3 | 1.40 |
| | carcinoma | | | | | |
| 85 | Infiltrating duct carcinoma | Black | F | 42 | 4 | 0.75 |
| 86 | Infiltrating duct carcinoma | White | F | 39 | 4 | 1.46 |
| 87 | Infiltrating duct carcinoma | White | F | 42 | 4 | 0.62 |
| 88 | Infiltrating duct carcinoma | White | F | 48 | 4 | 0.65 |
| 89 | Infiltrating duct carcinoma | White | F | 57 | 4 | 1.23 |
| 90 | Infiltrating duct carcinoma | White | F | 58 | 4 | 0.47 |
| 91 | Carcinoma | White | F | 68 | 4 | 1.06 |
| 92 | Infiltrating duct carcinoma | White | F | 65 | 4 | 1.13 |
| 93 | Infiltrating duct carcinoma | White | F | 73 | 4 | −0.07 |
| 94 | Infiltrating duct carcinoma | White | F | 77 | 4 | 1.48 |
| 95 | Infiltrating duct carcinoma | White | F | 53 | 4 | 2.67 |
| 96 | Infiltrating duct carcinoma | White | F | 40 | 4 | 0.37 |
| 97 | Infiltrating duct carcinoma | White | F | 53 | 4 | 5.76 |
| 98 | Infiltrating duct carcinoma | White | F | 66 | 4 | 4.41 |
| 99 | Infiltrating duct carcinoma | White | F | 70 | 4 | 0.38 |

Test set (5 normal controls and 25 cancer cases)

| | | | | | | |
|---|---|---|---|---|---|---|
| 100 | Normal control* | White | F | 77 | | −1.09 |
| 101 | Normal control* | White | F | 62 | | −0.47 |
| 102 | Normal control* | White | F | 48 | | −0.81 |
| 103 | Atypical ductal hyperplasia | White | F | 50 | Benign | 0.27 |
| 104 | Fibrocystic Change | White | F | 47 | Benign | −0.25 |
| 105 | Intraductal carcinoma, | White | F | 55 | 0 | 0.57 |
| | noninfiltrating, NOS | | | | | |
| 106 | Intraductal carcinoma, | White | F | 44 | 0 | 0.68 |
| | noninfiltrating, NOS | | | | | |
| 107 | Intraductal carcinoma, | Black | F | 76 | 0 | 1.26 |
| | noninfiltrating, NOS | | | | | |
| 108 | DCIS, NOS | White | F | 73 | 0 | 0.48 |
| 109 | DCIS, NOS | White | F | 69 | 0 | 1.45 |
| 110 | Infiltrating duct carcinoma, | White | F | 51 | 1 | 0.83 |
| | NOS | | | | | |
| 111 | Infiltrating duct carcinoma, | White | F | 72 | 1 | 1.08 |
| | NOS | | | | | |
| 112 | Infiltrating duct carcinoma, | White | F | 68 | 1A | 0.70 |
| | NOS | | | | | |
| 113 | Infiltrating duct carcinoma, | White | F | 50 | 1A | 0.50 |
| | NOS | | | | | |
| 114 | Infiltrating duct carcinoma, | White | F | 64 | 1A | 0.73 |
| | NOS | | | | | |
| 115 | Infiltrating duct carcinoma, | White | F | 62 | 2A | 1.26 |
| | NOS | | | | | |
| 116 | Infiltrating duct carcinoma, | White | F | 68 | 2A | 0.57 |
| | NOS | | | | | |
| 117 | Invasive carcinoma, no | White | F | 68 | 2A | 0.73 |
| | special type | | | | | |
| 118 | Invasive carcinoma, no | White | F | 35 | 2A | 0.91 |
| | special type | | | | | |
| 119 | Invasive carcinoma, no | White | F | 54 | 2B | 0.61 |
| | special type | | | | | |
| 120 | Lobular carcinoma, NOS | White | F | 78 | 3C | 1.40 |
| 121 | Lobular carcinoma, NOS | White | F | 76 | 3A | 0.59 |
| 122 | Lobular carcinoma, NOS | White | F | 52 | 3A | 1.38 |
| 123 | Infiltrating duct carcinoma, | Black | F | 75 | 3B | 0.52 |
| | NOS | | | | | |
| 124 | Infiltrating duct and | Black | F | 59 | 3A | 0.70 |
| | lobular carcinoma | | | | | |
| 125 | Infiltrating duct carcinoma, | Native | F | 41 | 4 | 0.90 |
| | NOS | Amer- | | | | |
| | | ican | | | | |
| 126 | Infiltrating duct carcinoma, | White | F | 55 | 4 | 0.32 |
| | NOS | | | | | |
| 127 | Invasive carcinoma, no | White | F | 61 | 4 | 0.73 |
| | special type | | | | | |
| 128 | Infiltrating duct carcinoma, | White | F | 64 | 4 | 0.70 |
| | NOS | | | | | |
| 129 | Invasive carcinoma, no | White | F | 51 | 4 | 0.81 |
| | special type | | | | | |

*These normal controls are the same normal controls #85, #86 and #87 in TABLE 5.

TABLE 10
Characteristics of colon cancer patients and controls.

| | | | | | | |
|---|---|---|---|---|---|---|
| | | | | | | Exosomal |
| | | | | | | TF-Ag-α |
| | | | Gen- | | | level |
| ID | Cancer Morphology | Race | der | Age | Stage | (A.U.) |

Training set (40 normal controls and 40 cancer cases)

| | | | | | | |
|---|---|---|---|---|---|---|
| 1 | Normal control | White | F | 65 | | 0.37 |
| 2 | Normal control | White | M | 59 | | −0.27 |
| 3 | Normal control | White | F | 70 | | 0.33 |
| 4 | Normal control | White | F | 50 | | −0.19 |
| 5 | Normal control | Black | F | 70 | | −0.72 |
| 6 | Normal control | Black | F | 60 | | 0.08 |
| 7 | Normal control | White | M | 76 | | 0.13 |
| 8 | Normal control | White | F | 77 | | −0.56 |
| 9 | Normal control | White | M | 52 | | −0.41 |
| 10 | Normal control | Black | M | 60 | | −0.66 |
| 11 | Normal control | White | F | 79 | | −1.59 |
| 12 | Normal control | White | F | 53 | | 0.30 |
| 13 | Normal control | White | F | 68 | | −0.45 |
| 14 | Normal control | White | M | 57 | | −0.03 |
| 15 | Normal control | White | M | 69 | | 0.38 |
| 16 | Normal control | White | F | 65 | | −1.30 |
| 17 | Normal control | Black | M | 69 | | −0.96 |
| 18 | Normal control | White | M | 62 | | −0.31 |
| 19 | Normal control | White | M | 66 | | −0.12 |
| 20 | Normal control | White | M | 51 | | −1.53 |
| 21 | Tubular Adenoma | White | F | 52 | Benign | 0.08 |
| 22 | Diverticular disease, | White | F | 62 | Benign | −0.62 |
| | diverticulosis | | | | | |
| 23 | Diverticular disease, | White | M | 79 | Benign | −0.69 |
| | diverticulosis | | | | | |
| 24 | Tubular Adenoma | White | F | 63 | Benign | −0.63 |
| 25 | Familial Adenomatous | White | F | 51 | Benign | −0.43 |
| | polyposis | | | | | |
| 26 | Tubulovillous Adenoma | White | M | 73 | Benign | 0.27 |
| 27 | Diverticular disease, | White | M | 60 | Benign | 0.04 |
| | diverticulosis | | | | | |
| 28 | Gardner Syndrome | White | M | 57 | Benign | −0.65 |
| 29 | Mucinous Cystadenoma | White | F | 47 | Benign | 0.02 |
| 30 | Polyposis Coli | White | M | 44 | Benign | −0.01 |
| 31 | Abdominal desmoid | White | M | 68 | Benign | 0.35 |
| | tumors | | | | | |
| 32 | Familial Adenomatous | White | M | 47 | Benign | −1.09 |
| | polyposis | | | | | |
| 33 | Tubulovillous Adenoma | White | F | 70 | Benign | −0.63 |
| 34 | Adenomatous polyp | Black | M | 53 | Benign | 0.32 |
| 35 | Hereditary nonpolyposis | White | F | 47 | Benign | −1.10 |
| | colorectal cancer | | | | | |
| | (HNPCC) | | | | | |
| 36 | Polyp of colon | White | M | 58 | Benign | −0.34 |
| 37 | HNPCC | White | F | 46 | Benign | −1.27 |
| 38 | Villous Adenoma | White | F | 30 | Benign | −0.21 |
| 39 | Mucinous tumor of low | White | M | 63 | Benign | −0.79 |
| | malignant potential | | | | | |
| 40 | Adenomatous polyp | White | F | 60 | Benign | 0.02 |
| 41 | Adenocarcinoma, NOS | White | F | 80 | 1 | 0.56 |
| 42 | Adenocarcinoma, NOS | White | F | 50 | 1 | 0.96 |
| 43 | Adenocarcinoma, NOS | Black | M | 64 | 1 | 1.12 |
| 44 | Adenocarcinoma, NOS | White | M | 51 | 1 | 0.50 |
| 45 | Adenocarcinoma in | Un- | F | 51 | 1 | 0.36 |
| | adenomatous polyp | known | | | | |
| 46 | Adenocarcinoma, NOS | White | F | 47 | 1 | 0.79 |
| 47 | Adenocarcinoma, NOS | White | M | 74 | 1 | 0.56 |
| 48 | Adenocarcinoma, NOS | White | M | 56 | 1 | 1.06 |
| 49 | Adenocarcinoma in | White | M | 53 | 1 | 0.56 |
| | tubular adenoma | | | | | |
| 50 | Adenocarcinoma, NOS | White | F | 77 | 1 | 0.52 |
| 51 | Adenocarcinoma, NOS | White | M | 68 | 2A | 0.45 |
| 52 | Adenocarcinoma, NOS | White | F | 60 | 2A | 0.41 |
| 53 | Adenocarcinoma, NOS | White | M | 91 | 2A | 0.48 |
| 54 | Adenocarcinoma, NOS | White | F | 49 | 2A | 0.53 |
| 55 | Adenocarcinoma, NOS | White | F | 80 | 2A | 0.49 |
| 56 | Adenocarcinoma, NOS | White | F | 87 | 2A | 1.05 |
| 57 | Gastrointestinal stromal | White | M | 74 | 2B | 0.52 |
| | sarcoma | | | | | |
| 58 | Adenocarcinoma, NOS | White | M | 67 | 2C | 0.65 |
| 59 | Mucinous | White | F | 71 | 2C | 0.61 |
| | adenocarcinoma | | | | | |
| 60 | Mucinous | White | F | 82 | 2C | 1.25 |
| | adenocarcinoma | | | | | |
| 61 | Adenocarcinoma, NOS | Un- | M | 59 | 3B | 0.56 |
| | | known | | | | |
| 62 | Adenocarcinoma, NOS | White | M | 65 | 3B | 0.85 |
| 63 | Adenocarcinoma, NOS | White | F | 84 | 3B | 0.85 |
| 64 | Adenocarcinoma, NOS | Native | F | 44 | 3B | 0.78 |
| | | Amer- | | | | |
| | | ican | | | | |
| 65 | Mucinous | White | M | 68 | 3B | 0.60 |
| | adenocarcinoma | | | | | |
| 66 | Adenocarcinoma, NOS | White | M | 65 | 3C | 0.90 |
| 67 | Adenocarcinoma in | White | F | 59 | 3A | 0.70 |
| | adenomatous polyp | | | | | |
| 68 | Adenocarcinoma in | White | M | 53 | 3A | 0.50 |
| | tubulovillous adenoma | | | | | |
| 69 | Adenocarcinoma, NOS | White | M | 56 | 3B | 1.14 |
| 70 | Adenocarcinoma, NOS | White | F | 67 | 3B | 2.71 |
| 71 | Adenocarcinoma in | White | M | 85 | 4 | 0.93 |
| | tubulovillous adenoma | | | | | |
| 72 | Signet ring cell | Un- | F | 50 | 4 | 1.24 |
| | carcinoma | known | | | | |
| 73 | Mucinous | White | M | 66 | 4A | 0.71 |
| | adenocarcinoma | | | | | |
| 74 | Adenocarcinoma, NOS | White | F | 62 | 4A | 0.91 |
| 75 | Adenocarcinoma, NOS | White | F | 69 | 4A | 0.85 |
| 76 | Adenocarcinoma, NOS | White | M | 63 | 4B | 1.18 |
| 77 | Adenocarcinoma, NOS | White | M | 69 | 4B | 0.91 |
| 78 | Small cell carcinoma, | White | M | 67 | 4B | 1.37 |
| | NOS | | | | | |
| 79 | Mucinous | White | F | 67 | 4C | 0.68 |
| | adenocarcinoma | | | | | |
| 80 | Adenocarcinoma, NOS | White | F | 55 | 4C | 1.11 |

Test set (29 normal controls and 20 cancer cases)

| | | | | | | |
|---|---|---|---|---|---|---|
| 81 | Normal control (low risk) | White | F | 50 | | −0.09 |
| 82 | Normal control (low risk) | White | M | 54 | | 0.07 |
| 83 | Normal control (low risk) | White | F | 70 | | −0.04 |
| 84 | Normal control (low risk) | Black | F | 66 | | −0.18 |
| 85 | Normal control (low risk) | Black | M | 58 | | −1.11 |
| 86 | Normal control (low risk) | Black | M | 51 | | −0.81 |
| 87 | Normal control (low risk) | Asian | M | 54 | | −1.23 |
| 88 | Normal control (low risk) | Black | F | 56 | | −0.29 |
| 89 | Normal control (low risk) | White | F | 55 | | −1.26 |
| 90 | Normal control (low risk) | White | M | 72 | | −0.63 |
| 91 | Normal control (low risk) | White | M | 67 | | −1.39 |
| 92 | Normal control (low risk) | White | F | 72 | | −0.35 |
| 93 | Normal control (low risk) | White | M | 77 | | −0.71 |
| 94 | Normal control (low risk) | White | M | 72 | | −1.43 |
| 95 | Normal control (low risk) | White | F | 57 | | 0.12 |
| 96 | Normal control (low risk) | White | F | 77 | | −0.44 |
| 97 | Normal control (low risk) | White | F | 52 | | 0.18 |
| 98 | Normal control (low risk) | White | F | 77 | | −1.63 |
| 99 | Normal control (low risk) | White | M | 55 | | −0.74 |
| 100 | Normal control (low risk) | White | F | 69 | | 0.20 |
| 101 | Normal control (low risk) | White | M | 72 | | 0.18 |
| 102 | Normal control (low risk) | White | M | 72 | | −0.25 |
| 103 | Normal control (low risk) | White | F | 58 | | −0.60 |
| 104 | Normal control (low risk) | White | M | 61 | | −0.19 |
| 105 | Normal control (low risk) | White | M | 55 | | 0.07 |
| 106 | Normal control (low risk) | White | F | 60 | | −1.20 |
| 107 | Normal control (low risk) | White | F | 67 | | −0.39 |
| 108 | Normal control (low risk) | White | F | 79 | | 0.08 |
| 109 | Normal control (low risk) | Asian | F | 69 | | −0.16 |
| 110 | Adenocarcinoma in | White | F | 45 | 1 | 1.01 |
| | villous adenoma | | | | | |
| 111 | Adenocarcinoma, NOS | White | F | 74 | 1 | 0.62 |
| 112 | Adenocarcinoma in | White | M | 57 | 1 | 0.62 |
| | adenomatous polyp | | | | | |
| 113 | Adenocarcinoma, NOS | Black | M | 61 | 1 | 0.90 |
| 114 | Adenocarcinoma in | White | F | 50 | 1 | 0.84 |
| | tubulovillous adenoma | | | | | |
| 115 | Adenocarcinoma in | White | F | 79 | 2A | 1.91 |
| | polypoid adenoma | | | | | |
| 116 | Adenocarcinoma in | White | M | 54 | 2A | 0.56 |
| | tubulovillous adenoma | | | | | |
| 117 | Adenocarcinoma, NOS | White | M | 74 | 2A | 2.17 |
| 118 | Adenocarcinoma, NOS | White | F | 58 | 2A | 0.82 |
| 119 | Adenocarcinoma with | White | F | 68 | 2B | 0.63 |
| | mixed subtypes | | | | | |
| 120 | Adenocarcinoma in | White | F | 56 | 3A | 0.56 |
| | tubulovillous adenoma | | | | | |
| 121 | Adenocarcinoma, NOS | White | F | 55 | 3B | 0.56 |
| 122 | Adenocarcinoma, NOS | White | M | 69 | 3C | 0.64 |
| 123 | Adenocarcinoma, NOS | White | M | 66 | 3C | 1.56 |
| 124 | Adenocarcinoma, NOS | White | M | 56 | 3C | 1.28 |
| 125 | Adenocarcinoma, NOS | White | M | 56 | 4 | 0.86 |
| 126 | Adenocarcinoma in | White | F | 52 | 4A | 0.66 |
| | tubulovillous adenoma | | | | | |
| 127 | Adenocarcinoma in | White | M | 63 | 4A | 0.58 |
| | tubular adenoma | | | | | |
| 128 | Adenocarcinoma, NOS | White | M | 65 | 4B | 1.09 |
| 129 | Adenocarcinoma, NOS | White | F | 52 | 4C | 1.19 |

TABLE 11
Characteristics of ovarian cancer patients and controls.

| | | | | | | Exosomal | Serum |
|---|---|---|---|---|---|---|---|
| | | | | | | TF-Ag-α | CA125 |
| | | | | | | level | level |
| ID | Cancer Morphology | Race | Gender | Age | Stage | (A.U.) | (U/mL) |
| Training set (30 normal controls and 40 cancer cases) | | | | | | | |
| *1 | Normal control | White | F | 65 | | 0.37 | N/A |
| *2 | Normal control | White | F | 70 | | 0.33 | N/A |
| *3 | Normal control | White | F | 50 | | −0.19 | N/A |
| *4 | Normal control | Black | F | 70 | | −0.72 | N/A |
| *5 | Normal control | Black | F | 60 | | 0.08 | N/A |
| *6 | Normal control | White | F | 77 | | −0.56 | N/A |
| *7 | Normal control | White | F | 79 | | −1.59 | N/A |
| *8 | Normal control | White | F | 53 | | 0.30 | N/A |
| *9 | Normal control | White | F | 68 | | −0.45 | N/A |
| *10 | Normal control | White | F | 65 | | −1.30 | N/A |
| 11 | Dermoid Cyst | White | F | 41 | Benign | 0.34 | N/A |
| 12 | Mucinous Cystadenoma | White | F | 56 | Benign | 0.20 | N/A |
| 13 | Mucinous Cystadenoma | White | F | 62 | Benign | 0.37 | N/A |
| 14 | Corpus Luteum Cyst | White | F | 47 | Benign | −1.08 | N/A |
| 15 | Serous Cystadenoma | White | F | 60 | Benign | −0.30 | N/A |
| 16 | Serous Cystadenoma | White | F | 53 | Benign | −0.74 | N/A |
| 17 | Serous Cyst | White | F | 57 | Benign | −0.25 | N/A |
| 18 | Corpus Luteum Cyst | White | F | 47 | Benign | −0.86 | N/A |
| 19 | Cystadenoma | White | F | 54 | Benign | 0.02 | N/A |
| 20 | Serous Cyst | White | F | 65 | Benign | 0.38 | N/A |
| 21 | Mucinous Cystadenoma | White | F | 56 | Benign | −0.51 | N/A |
| 22 | Endometrioma | White | F | 38 | Benign | 0.23 | N/A |
| 23 | Serous Cystadenoma | White | F | 54 | Benign | −0.88 | N/A |
| 24 | Serous Cyst | White | F | 85 | Benign | −0.51 | N/A |
| 25 | Endometrioma | White | F | 47 | Benign | 0.15 | 64.10 |
| 26 | Dermoid Cyst | White | F | 40 | Benign | 0.23 | 12.40 |
| 27 | Follicular Cyst | Black | F | 45 | Benign | 0.29 | 31.30 |
| 28 | Cystadenoma | White | F | 69 | Benign | −0.01 | N/A |
| 29 | Serous Cystadenoma | White | F | 61 | Benign | −1.21 | N/A |
| 30 | Follicular Cyst | White | F | 52 | Benign | −0.08 | N/A |
| 31 | Serous cystadenoma, | White | F | 67 | 1A | 0.60 | 7.3 |
| | borderline malig | | | | | | |
| 32 | Endometrioid | White | F | 54 | 1A | 0.87 | 11.0 |
| | adenocarcinoma, NOS | | | | | | |
| 33 | Carcinosarcoma, NOS | White | F | 73 | 1A | 0.83 | 9.0 |
| 34 | Papillary transitional | White | F | 65 | 1A | 0.51 | 4.8 |
| | cell carcinoma | | | | | | |
| 35 | Serous surface papillary | White | F | 63 | 1B | 1.29 | 7.5 |
| | carcinoma (C56.9) | | | | | | |
| 36 | Clear cell carcinoma | White | F | 52 | 1C | 0.56 | 46.6 |
| 37 | Mucinous | White | F | 57 | 1C | 0.54 | 211.3 |
| | cystadenocarcinoma, | | | | | | |
| | NOS | | | | | | |
| 38 | Mucinous carcinoma | White | F | 70 | 1C | 0.61 | 30.1 |
| 39 | Adult granulosa cell | White | F | 68 | 1C | 0.48 | N/A |
| | tumor of ovary | | | | | | |
| 40 | Mucinous | White | F | 54 | 1C | 1.29 | 160.3 |
| | cystadenocarcinoma, | | | | | | |
| | NOS | | | | | | |
| 41 | Adenocarcinoma, NOS | White | F | 53 | 2A | 0.85 | 99.8 |
| 42 | Serous surface papillary | White | F | 84 | 2A | 1.54 | 23.6 |
| | carcinoma (C56.9) | | | | | | |
| 43 | Serous adenocarcinoma, | White | F | 77 | 2A | 0.86 | 333.1 |
| | NOS (C56.9) | | | | | | |
| 44 | Mixed cell | White | F | 55 | 2B | 1.33 | 211.8 |
| | adenocarcinoma | | | | | | |
| 45 | Serous carcinoma, NOS | Black | F | 59 | 2C | 1.00 | 102.2 |
| 46 | Serous carcinoma, NOS | White | F | 60 | 2C | 0.63 | 23.3 |
| 47 | Papillary serous | White | F | 40 | 2C | 0.78 | 9.5 |
| | cystadenocarcinoma | | | | | | |
| 48 | Clear cell | White | F | 67 | 2B | 2.90 | N/A |
| | adenocarcinoma, NOS | | | | | | |
| 49 | Clear cell | White | F | 55 | 2B | 0.73 | 33.5 |
| | adenocarcinoma, NOS | | | | | | |
| 50 | Serous surface papillary | White | F | 77 | 2B | 0.49 | 54.0 |
| | carcinoma (C56.9) | | | | | | |
| 51 | Serous surface papillary | White | F | 60 | 3B | 0.58 | N/A |
| | carcinoma (C56.9) | | | | | | |
| 52 | Serous surface papillary | White | F | 68 | 3C | 1.53 | N/A |
| | carcinoma (C56.9) | | | | | | |
| 53 | Carcinosarcoma, NOS | White | F | 67 | 3C | 0.49 | 5.1 |
| 54 | Serous | White | F | 58 | 3C | 1.69 | 107.0 |
| | cystadenocarcinoma, | | | | | | |
| | NOS (C56.9) | | | | | | |
| 55 | Serous surface papillary | White | F | 82 | 3C | 1.14 | N/A |
| | carcinoma | | | | | | |
| 56 | Serous surface papillary | White | F | 76 | 3C | 2.39 | 167.6 |
| | carcinoma (C56.9) | | | | | | |
| 57 | Mixed cell | White | F | 89 | 3C | 0.70 | N/A |
| | adenocarcinoma | | | | | | |
| 58 | Mixed cell | White | F | 75 | 3C | 0.59 | 74.7 |
| | adenocarcinoma | | | | | | |
| 59 | Serous surface papillary | White | F | 59 | 3C | 0.58 | 12.1 |
| | carcinoma | | | | | | |
| 60 | Serous surface papillary | White | F | 76 | 3C | 0.73 | 131.1 |
| | carcinoma (C56.9) | | | | | | |
| 61 | High grade serous | White | F | 65 | 4 | 0.70 | N/A |
| | carcinoma | | | | | | |
| 62 | Mixed cell | White | F | 61 | 4 | 1.45 | 30.4 |
| | adenocarcinoma | | | | | | |
| 63 | Papillary serous | White | F | 68 | 4 | 0.69 | 165.6 |
| | cystadenocarcinoma | | | | | | |
| 64 | Mixed cell | White | F | 54 | 4 | 1.22 | 104.4 |
| | adenocarcinoma | | | | | | |
| 65 | Serous | White | F | 62 | 4 | 0.77 | 12.3 |
| | cystadenocarcinoma, | | | | | | |
| | NOS (C56.9) | | | | | | |
| 66 | Papillary serous | White | F | 68 | 4 | 1.51 | 9.2 |
| | cystadenocarcinoma | | | | | | |
| 67 | Mesodermal mixed | White | F | 78 | 4 | 0.86 | 2847.3 |
| | tumor | | | | | | |
| 68 | Serous surface papillary | White | F | 77 | 4 | 0.73 | 218.9 |
| | carcinoma (C56.9) | | | | | | |
| 69 | Serous surface papillary | White | F | 65 | 4 | 0.75 | 361.8 |
| | carcinoma (C56.9) | | | | | | |
| 70 | Serous surface papillary | White | F | 51 | 4B | 2.29 | 8399.2 |
| | carcinoma | | | | | | |

Test set (16 normal controls and 20 cancer cases)

| | | | | | | | |
|---|---|---|---|---|---|---|---|
| *71 | Normal control (low risk) | White | F | 50 | | −0.09 | N/A |
| *72 | Normal control (low risk) | White | F | 70 | | −0.04 | N/A |
| *73 | Normal control (low risk) | Black | F | 66 | | −0.18 | N/A |
| *74 | Normal control (low risk) | Black | F | 56 | | −0.29 | N/A |
| *75 | Normal control (low risk) | White | F | 55 | | −1.26 | N/A |
| *76 | Normal control (low risk) | White | F | 72 | | −0.35 | N/A |
| *77 | Normal control (low risk) | White | F | 57 | | 0.12 | N/A |
| *78 | Normal control (low risk) | White | F | 77 | | −0.44 | N/A |
| *79 | Normal control (low risk) | White | F | 52 | | 0.18 | N/A |
| *80 | Normal control (low risk) | White | F | 77 | | −1.63 | N/A |
| *81 | Normal control (low risk) | White | F | 69 | | 0.20 | N/A |
| *82 | Normal control (low risk) | White | F | 58 | | −0.60 | N/A |
| *83 | Normal control (low risk) | White | F | 60 | | −1.20 | N/A |
| *84 | Normal control (low risk) | White | F | 67 | | −0.39 | N/A |
| *85 | Normal control (low risk) | White | F | 79 | | 0.08 | N/A |
| *86 | Normal control (low risk) | Asian | F | 69 | | −0.16 | N/A |
| 87 | Mixed cell | White | F | 57 | 1A | 0.55 | 7.0 |
| | adenocarcinoma | | | | | | |
| 88 | Endometrioid | White | F | 60 | 1A | 0.53 | 9.4 |
| | adenocarcinoma, NOS | | | | | | |
| 89 | Mixed cell | White | F | 48 | 1C | 0.45 | 19.6 |
| | adenocarcinoma | | | | | | |
| 90 | Serous tumor, NOS, low | White | F | 48 | 1C | 0.51 | 45.6 |
| | malignant potential | | | | | | |
| 91 | Serous | White | F | 67 | 1C | 0.67 | 111.7 |
| | cystadenocarcinoma, | | | | | | |
| | NOS (C56.9) | | | | | | |
| 92 | Endometrioid carcinoma, | White | F | 61 | 2A | 0.51 | 517.2 |
| | NOS | | | | | | |
| 93 | Mixed cell | White | F | 52 | 2A | 0.56 | 11.2 |
| | adenocarcinoma | | | | | | |
| 94 | Clear cell carcinoma | White | F | 62 | 2B | 0.52 | 110.8 |
| 95 | Endometrioid | White | F | 59 | 2B | 0.72 | 133.0 |
| | adenocarcinoma, NOS | | | | | | |
| 96 | Serous surface papillary | White | F | 65 | 2C | 2.29 | 19.2 |
| | carcinoma (C56.9) | | | | | | |
| 97 | Papillary serous | White | F | 76 | 3C | 0.52 | N/A |
| | cystadenocarcinoma | | | | | | |
| 98 | Serous adenocarcinoma, | White | F | 52 | 3C | 0.56 | 836.6 |
| | NOS (C56.9) | | | | | | |
| 99 | Endometrioid | White | F | 47 | 3C | 1.22 | 160.4 |
| | adenocarcinoma, NOS | | | | | | |
| 100 | Serous surface papillary | White | F | 54 | 3C | 0.95 | 991.6 |
| | carcinoma (C56.9) | | | | | | |
| 101 | Mixed cell | White | F | 50 | 3C | 0.65 | N/A |
| | adenocarcinoma | | | | | | |
| 102 | Carcinosarcoma, NOS | White | F | 64 | 4 | 1.37 | 81.8 |
| 103 | Serous surface papillary | White | F | 68 | 4 | 0.88 | 14.1 |
| | carcinoma | | | | | | |
| 104 | Serous surface papillary | White | F | 56 | 4 | 0.50 | 54.3 |
| | carcinoma | | | | | | |
| 105 | Mucinous | Black | F | 60 | 4 | 0.59 | N/A |
| | adenocarcinoma | | | | | | |
| 106 | Serous | White | F | 65 | 4 | 1.28 | 6.4 |
| | cystadenocarcinoma, | | | | | | |
| | NOS (C56.9) | | | | | | |

*These normal controls are the same normal controls #1, #3-6, #8, #11-13, #16, #81, #83, #84, #88, #89, #92, #95-98, #100, #103, #106-109 in TABLE 10.

TABLE 12
Characteristics of prostate cancer and controls.

| | | | | | | | |
|---|---|---|---|---|---|---|---|
| | | | | | | Exosomal | Serum |
| | | | | | | TF-Ag-α | PSA |
| | Cancer | | | | | level | level |
| ID | Morphology | Race | Gender | Age | Stage | (A.U.) | (ng/mL) |

Training set (30 normal controls and 40 cancer cases)

| | | | | | | | |
|---|---|---|---|---|---|---|---|
| *1 | Normal control | White | M | 59 | | −0.27 | N/A |
| *2 | Normal control | White | M | 76 | | 0.13 | N/A |
| *3 | Normal control | White | M | 52 | | −0.41 | N/A |
| *4 | Normal control | Black | M | 60 | | −0.66 | N/A |
| *5 | Normal control | White | M | 57 | | −0.03 | N/A |
| *6 | Normal control | White | M | 69 | | 0.38 | N/A |
| *7 | Normal control | Black | M | 69 | | −0.96 | N/A |
| *8 | Normal control | White | M | 62 | | −0.31 | N/A |
| *9 | Normal control | White | M | 66 | | −0.12 | N/A |
| *10 | Normal control | White | M | 51 | | −1.53 | N/A |
| 11 | Benign prostatic | White | M | 65 | Benign | −0.28 | N/A |
| | hyperplasia (BPH) | | | | | | |
| 12 | High-grade prostatic | White | M | 64 | Benign | −0.32 | <0.03 |
| | intraepithelial | | | | | | |
| | neoplasia (HGPIN) | | | | | | |
| | (PIN III) | | | | | | |
| 13 | Focal glandular | White | M | 57 | Benign | −0.26 | N/A |
| | hyperplasia | | | | | | |
| 14 | HGPIN (PIN III) | White | M | 63 | Benign | −0.50 | 5.11 |
| 15 | HGPIN (PIN III) | White | M | 70 | Benign | −0.90 | 31.69 |
| 16 | Phyllodes tumor, | White | M | 70 | Benign | −0.39 | <0.03 |
| | benign | | | | | | |
| 17 | Prostatitis | White | M | 55 | Benign | 0.40 | N/A |
| 18 | HGPIN (PIN III) | White | M | 60 | Benign | −0.88 | 3.06 |
| 19 | HGPIN (PIN III) | Black | M | 72 | Benign | −0.73 | 10.41 |
| 20 | Focal glandular | White | M | 64 | Benign | −0.35 | 6.69 |
| | hyperplasia | | | | | | |
| 21 | BPH | Unknown | M | 75 | Benign | −0.72 | 6.82 |
| 22 | BPH | White | M | 63 | Benign | 0.29 | 13.80 |
| 23 | BPH | White | M | 69 | Benign | −1.36 | 0.96 |
| 24 | BPH | White | M | 60 | Benign | −0.12 | 13.58 |
| 25 | BPH | White | M | 74 | Benign | −0.57 | N/A |
| 26 | BPH | White | M | 53 | Benign | −0.22 | 4.26 |
| 27 | BPH | White | M | 73 | Benign | −0.77 | N/A |
| 28 | HGPIN (PIN III) | White | M | 59 | Benign | 0.36 | 0.80 |
| 29 | Prostatitis | White | M | 78 | Benign | −0.76 | 3.64 |
| 30 | BPH | White | M | 60 | Benign | −0.97 | 2.30 |
| 31 | Adenocarcinoma, | White | M | 55 | 1 | 0.89 | 0.04 |
| | NOS | | | | | | |
| 32 | Adenocarcinoma, | White | M | 63 | 1 | 0.63 | <0.04 |
| | NOS | | | | | | |
| 33 | Adenocarcinoma, | Asian | M | 71 | 1 | 0.89 | <0.03 |
| | NOS | | | | | | |
| 34 | Adenocarcinoma, | White | M | 49 | 1 | 1.39 | 0.03 |
| | NOS | | | | | | |
| 35 | Acinar | White | M | 58 | 1 | 0.75 | <0.04 |
| | adenocarcinoma | | | | | | |
| | (C61.9 ONLY) (For | | | | | | |
| | prostate only, do not | | | | | | |
| | use 8550/3) | | | | | | |
| 36 | Adenocarcinoma, | White | M | 75 | 1 | 1.26 | <0.03 |
| | NOS | | | | | | |
| 37 | Adenocarcinoma, | White | M | 55 | 1 | 0.74 | 4.11 |
| | NOS | | | | | | |
| 38 | Adenocarcinoma, | White | M | 58 | 1 | 0.72 | <0.03 |
| | NOS | | | | | | |
| 39 | Adenocarcinoma, | White | M | 65 | 1 | 1.30 | <0.04 |
| | NOS | | | | | | |
| 40 | Adenocarcinoma, | White | M | 58 | 1 | 1.01 | N/A |
| | NOS | | | | | | |
| 41 | Adenocarcinoma, | White | M | 62 | 2 | 0.70 | <0.03 |
| | NOS | | | | | | |
| 42 | Adenocarcinoma, | White | M | 51 | 2 | 0.80 | N/A |
| | NOS | | | | | | |
| 43 | Adenocarcinoma, | White | M | 54 | 2B | 0.75 | 0.16 |
| | NOS | | | | | | |
| 44 | Adenocarcinoma, | White | M | 69 | 2B | 0.87 | <0.04 |
| | NOS | | | | | | |
| 45 | Adenocarcinoma, | White | M | 55 | 2B | 1.00 | <0.03 |
| | NOS | | | | | | |
| 46 | Adenocarcinoma, | White | M | 56 | 2B | 1.69 | <0.04 |
| | NOS | | | | | | |
| 47 | Acinar | White | M | 68 | 2B | 0.81 | <0.04 |
| | adenocarcinoma | | | | | | |
| | (C61.9 ONLY) (For | | | | | | |
| | prostate only, do not | | | | | | |
| | use 8550/3) | | | | | | |
| 48 | Adenocarcinoma, | White | M | 67 | 2B | 1.19 | N/A |
| | NOS | | | | | | |
| 49 | Adenocarcinoma, | White | M | 59 | 2B | 0.90 | <0.03 |
| | NOS | | | | | | |
| 50 | Adenocarcinoma, | White | M | 51 | 2B | 2.50 | 0.05 |
| | NOS | | | | | | |
| 51 | Adenocarcinoma, | White | M | 69 | 3 | 1.35 | 0.15 |
| | NOS | | | | | | |
| 52 | Adenocarcinoma, | White | M | 66 | 3 | 1.05 | <0.03 |
| | NOS | | | | | | |
| 53 | Acinar | White | M | 50 | 3A | 1.48 | <0.04 |
| | adenocarcinoma | | | | | | |
| | (C61.9 ONLY) (For | | | | | | |
| | prostate only, do not | | | | | | |
| | use 8550/3) | | | | | | |
| 54 | Adenocarcinoma, | White | M | 60 | 3A | 1.13 | <0.04 |
| | NOS | | | | | | |
| 55 | Adenocarcinoma, | White | M | 62 | 3B | 1.41 | 4.76 |
| | NOS | | | | | | |
| 56 | Adenocarcinoma, | White | M | 62 | 3B | 1.25 | 14.82 |
| | NOS | | | | | | |
| 57 | Adenocarcinoma, | White | M | 52 | 3B | 0.99 | 0.07 |
| | NOS | | | | | | |
| 58 | Adenocarcinoma, | White | M | 56 | 3B | 1.32 | 39.57 |
| | NOS | | | | | | |
| 59 | Adenocarcinoma, | White | M | 62 | 3C | 1.04 | 6.92 |
| | NOS | | | | | | |
| 60 | Adenocarcinoma, | White | M | 47 | 3C | 1.06 | 1.78 |
| | NOS | | | | | | |
| 61 | Adenocarcinoma, | White | M | 45 | 4 | 1.25 | 323.39 |
| | NOS | | | | | | |
| 62 | Adenocarcinoma, | Unknown | M | 71 | 4B | 1.07 | 189.44 |
| | NOS | | | | | | |
| 63 | Adenocarcinoma, | White | M | 81 | 4B | 2.71 | 976.64 |
| | NOS | | | | | | |
| 64 | Adenocarcinoma, | White | M | 47 | 4B | 1.09 | 3.34 |
| | NOS | | | | | | |
| 65 | Acinar | White | M | 62 | 4A | 0.57 | 0.17 |
| | adenocarcinoma | | | | | | |
| 66 | Adenocarcinoma, | White | M | 58 | 4A | 1.50 | N/A |
| | NOS | | | | | | |
| 67 | Adenocarcinoma, | White | M | 61 | 4A | 1.03 | 7.97 |
| | NOS | | | | | | |
| 68 | Adenocarcinoma, | White | M | 67 | 4 | 0.62 | 4.49 |
| | NOS | | | | | | |
| 69 | Adenocarcinoma, | White | M | 58 | 4 | 0.67 | N/A |
| | NOS | | | | | | |
| 70 | Adenocarcinoma, | White | M | 60 | 4 | 1.37 | 0.17 |
| | NOS | | | | | | |

Test set (13 normal controls and 20 cancer cases)

| | | | | | | | |
|---|---|---|---|---|---|---|---|
| *71 | Normal control (low | White | M | 54 | | 0.07 | N/A |
| | risk) | | | | | | |
| *72 | Normal control (low | Black | M | 58 | | −1.11 | N/A |
| | risk) | | | | | | |
| *73 | Normal control (low | Black | M | 51 | | −0.81 | N/A |
| | risk) | | | | | | |
| *74 | Normal control (low | Asian | M | 54 | | −1.23 | N/A |
| | risk) | | | | | | |
| *75 | Normal control (low | White | M | 72 | | −0.63 | N/A |
| | risk) | | | | | | |
| *76 | Normal control (low | White | M | 67 | | −1.39 | N/A |
| | risk) | | | | | | |
| *77 | Normal control (low | White | M | 77 | | −0.71 | N/A |
| | risk) | | | | | | |
| *78 | Normal control (low | White | M | 72 | | −1.43 | N/A |
| | risk) | | | | | | |
| *79 | Normal control (low | White | M | 55 | | −0.74 | N/A |
| | risk) | | | | | | |
| *80 | Normal control (low | White | M | 72 | | 0.18 | N/A |
| | risk) | | | | | | |
| *81 | Normal control (low | White | M | 72 | | −0.25 | N/A |
| | risk) | | | | | | |
| *82 | Normal control (low | White | M | 61 | | −0.19 | N/A |
| | risk) | | | | | | |
| *83 | Normal control (low | White | M | 55 | | 0.07 | N/A |
| | risk) | | | | | | |
| 84 | Adenocarcinoma, | White | M | 57 | 1 | 0.65 | <0.04 |
| | NOS | | | | | | |
| 85 | Adenocarcinoma, | White | M | 74 | 1 | 0.57 | <0.03 |
| | NOS | | | | | | |
| 86 | Adenocarcinoma, | White | M | 71 | 1 | 0.82 | <0.04 |
| | NOS | | | | | | |
| 87 | Acinar | Black | M | 68 | 1 | 1.26 | <0.04 |
| | adenocarcinoma | | | | | | |
| 88 | Adenocarcinoma, | White | M | 61 | 1 | 1.11 | 0.17 |
| | NOS | | | | | | |
| 89 | Adenocarcinoma, | White | M | 59 | 2 | 2.03 | N/A |
| | NOS | | | | | | |
| 90 | Adenocarcinoma, | White | M | 56 | 2 | 1.74 | 0.03 |
| | NOS | | | | | | |
| 91 | Adenocarcinoma, | White | M | 66 | 2B | 0.72 | N/A |
| | NOS | | | | | | |
| 92 | Adenocarcinoma, | White | M | 57 | 2B | 0.54 | <0.04 |
| | NOS | | | | | | |
| 93 | Adenocarcinoma, | Unknown | M | 61 | 2B | 1.49 | N/A |
| | NOS | | | | | | |
| 94 | Adenocarcinoma, | Black | M | 55 | 3 | 1.13 | <0.04 |
| | NOS | | | | | | |
| 95 | Adenocarcinoma, | White | M | 59 | 3 | 0.86 | N/A |
| | NOS | | | | | | |
| 96 | Adenocarcinoma, | Black | M | 58 | 3B | 0.49 | <0.04 |
| | NOS | | | | | | |
| 97 | Adenocarcinoma, | White | M | 69 | 3B | 0.86 | <0.04 |
| | NOS | | | | | | |
| 98 | Adenocarcinoma, | White | M | 58 | 3C | 0.52 | <0.04 |
| | NOS | | | | | | |
| 99 | Infiltrating duct | White | M | 59 | 4 | 1.07 | N/A |
| | carcinoma, NOS | | | | | | |
| 100 | Adenocarcinoma, | White | M | 63 | 4 | 2.10 | N/A |
| | NOS | | | | | | |
| 101 | Adenocarcinoma, | White | M | 53 | 4 | 0.50 | N/A |
| | NOS | | | | | | |
| 102 | Adenocarcinoma, | White | M | 72 | 4 | 0.74 | <0.04 |
| | NOS | | | | | | |
| 103 | Adenocarcinoma, | White | M | 58 | 4 | 0.79 | <0.03 |
| | NOS | | | | | | |

*These normal controls are the same normal controls #2, #7, #9, #10, #14, #15, #17-20, #82, #85-87, #90, #91, #93, #94, #99, #101, #102, #104, #105 in TABLE 10.

TABLE 14
Characteristics of nonsmall cell lung cancer patients receiving PD-1 checkpoint
inhibitors.

| | | | | | | |
|---|---|---|---|---|---|---|
| | | | | | | Tissue |
| | | Therapeutic | | Age | | PD-L1 |
| No. | Response* | regimen | Cancer subtype | Gender | Stage | TPS |

Discovery Set

| | | | | | | |
|---|---|---|---|---|---|---|
| 1 | Responder | Pembrolizumab | Adenocarcinoma | 66M | IV | 0% |
| | | Ramucirumab | | | | |
| 2 | Responder | Pembrolizumab | Squamous | 66M | IV | >50% |
| | | Carboplatin | | | | |
| | | Paclitaxel | | | | |
| 3 | Responder | Pembrolizumab | Adenocarcinoma | 72F | IV | 1% |
| | | Carboplatin | | | | |
| | | Pemetrexed | | | | |
| 4 | Responder | Pembrolizumab | Adenocarcinoma | 62M | IV | 20% |
| | | Carboplatin | | | | |
| | | Pemetrexed | | | | |
| 5 | Responder | Pembrolizumab | Adenocarcinoma | 77F | IV | 90% |
| 6 | Responder | Nivolumab | Squamous | 58M | IIIA | 0% |
| | | Carboplatin | | | | |
| | | Paclitaxel | | | | |
| 7 | Responder | Pembrolizumab | Adenocarcinoma | 79F | IV | >50% |
| 8 | Responder | Pembrolizumab | Squamous | 60F | III | 0% |
| | | Carboplatin | | | | |
| | | Paclitaxel | | | | |
| 9 | Responder | Nivolumab | Adenocarcinoma | 59F | IIIA | 1-49% |
| | | Carboplatin | | | | |
| | | Pemetrexed | | | | |
| 10 | Responder | Nivolumab | Adenocarcinoma | 69M | IV | 0% |
| | | Ipilimumab | | | | |
| | | Carboplatin | | | | |
| | | Pemetrexed | | | | |
| 11 | Responder | Pembrolizumab | Adenocarcinoma | 66M | IV | 1-49% |
| | | Carboplatin | | | | |
| | | Pemetrexed | | | | |
| 12 | Responder | Cemiplimab | Adenocarcinoma | 66F | IV | N/A |
| 13 | Responder | Nivolumab | Adenocarcinoma | 66F | IIIA | >50% |
| | | Cisplatin | | | | |
| | | Pemetrexed | | | | |
| 14 | Responder | Pembrolizumab | Squamous | 64M | IV | >50% |
| | | Carboplatin | | | | |
| | | Paclitaxel | | | | |
| 15 | Responder | Pembrolizumab | Squamous | 66F | IV | 1-49% |
| | | Carboplatin | | | | |
| | | Paclitaxel | | | | |
| 16 | Non-Responder | Pembrolizumab | Adenocarcinoma | 84M | IV | 10% |
| 17 | Non-Responder | Pembrolizumab | Adenocarcinoma | 76F | IV | >50% |
| 18 | Non-Responder | Pembrolizumab | Adenocarcinoma | 72F | IV | 1-49% |
| | | Pemetrexed | | | | |
| 19 | Non-Responder | Nivolumab | Adenocarcinoma | 59F | IV | 0% |
| 20 | Non-Responder | Nivolumab | Squamous | 75F | IV | 0% |
| | | Ipilimumab | | | | |
| 21 | Non-Responder | Pembrolizumab | Adenocarcinoma | 67F | IV | >50% |
| 22 | Non-Responder | Pembrolizumab | Adenocarcinoma | 67F | IV | 0% |
| | | Carboplatin | | | | |
| | | Pemetrexed | | | | |
| 23 | Non-Responder | Pembrolizumab | Adenocarcinoma | 72F | IV | 0% |
| | | Carboplatin | | | | |
| | | Pemetrexed | | | | |
| 24 | Non-Responder | Pembrolizumab | Squamous | 57M | IV | 1-49% |
| | | Carboplatin | | | | |
| | | Paclitaxel | | | | |
| 25 | Non-Responder | Pembrolizumab | Adenocarcinoma | 69M | IV | 0% |
| | | Carboplatin | | | | |
| | | Pemetrexed | | | | |
| 26 | Non-Responder | Cisplatin | Adenocarcinoma | 64M | IIIA | 0% |
| | | Pemetrexed | (colloid) | | | |
| | | Pembrolizumab | | | | |
| 27 | Non-Responder | Nivolumab | Squamous | 60M | IV | 0% |
| | | Ipilimumab | | | | |
| | | Carboplatin | | | | |
| | | Paclitaxel | | | | |
| 28 | Non-Responder | Pembrolizumab | Adenocarcinoma | 76M | IV | 1-49% |
| | | Carboplatin | | | | |
| 29 | Non-Responder | Pembrolizumab | Adenocarcinoma | 63M | IV | 0% |
| | | Carboplatin | | | | |
| | | Pemetrexed | | | | |

Validation Set

| | | | | | | |
|---|---|---|---|---|---|---|
| 30 | Responder | Pembrolizumab | Adenocarcinoma | 57F | IV | 70% |
| 31 | Responder | Pembrolizumab | Adenocarcinoma | 84M | IV | 60% |
| 32 | Responder | Pembrolizumab | Adenocarcinoma | 49F | IV | 60% |
| 33 | Responder | Pembrolizumab | Adenocarcinoma | 79F | IV | 50% |
| 34 | Responder | Pembrolizumab | Adenocarcinoma | 75F | IV | 90% |
| 35 | Responder | Pembrolizumab | Squamous | 61M | IV | >50% |
| 36 | Responder | Pembrolizumab | Adenocarcinoma | 88F | IIIA | 50% |
| 37 | Responder | Pembrolizumab | Adenocarcinoma | 55M | IV | 100% |
| 38 | Responder | Pembrolizumab | Squamous | 77F | IIIB | 1-49% |
| 39 | Responder | Pembrolizumab | Adenocarcinoma | 52M | IV | >75% |
| 40 | Responder | Pembrolizumab | Adenocarcinoma | 66M | IV | 100% |
| 41 | Responder | Pembrolizumab | Adenocarcinoma | 63F | III | >50% |
| 42 | Responder | Pembrolizumab | NSCLC with | 68M | IV | 100% |
| | | | giant cell | | | |
| | | | features | | | |
| 43 | Responder | Pembrolizumab | Squamous | 64F | IIIA | 80% |
| 44 | Responder | Pembrolizumab | Adenocarcinoma | 56F | IV | 0% |
| | | Carboplatin | | | | |
| | | Pemetrexed | | | | |
| 45 | Responder | Pembrolizumab | Adenocarcinoma | 71F | IIIB | 1-25% |
| | | Carboplatin | | | | |
| | | Pemetrexed | | | | |
| 46 | Responder | Pembrolizumab | Adenocarcinoma | 68M | IVB | 20% |
| | | Carboplatin | | | | |
| | | Pemetrexed | | | | |
| 47 | Responder | Pembrolizumab | Adenocarcinoma | 65F | IIIA | 1-25% |
| | | Carboplatin | | | | |
| | | Pemetrexed | | | | |
| 48 | Responder | Pembrolizumab | Adenocarcinoma | 61M | IV | >50% |
| | | Carboplatin | | | | |
| | | Pemetrexed | | | | |
| 49 | Responder | Pembrolizumab | Adenocarcinoma | 39M | IV | 100% |
| | | Carboplatin | | | | |
| | | Pemetrexed | | | | |
| 50 | Responder | Pembrolizumab | Squamous | 57F | IV | <1% |
| | | Carboplatin | | | | |
| | | Pemetrexed | | | | |
| 51 | Responder | Pembrolizumab | Squamous | 65M | IIIB | <1% |
| | | Carboplatin | | | | |
| | | Paclitaxel | | | | |
| 52 | Responder | Pembrolizumab | Adenocarcinoma | 66M | IV | 0% |
| | | Ramucirumab | | | | |
| 53 | Responder | Pembrolizumab | Adenocarcinoma | 77F | IV | 90% |
| 54 | Responder | Pembrolizumab | Adenocarcinoma | 79F | IV | >50% |
| 55 | Non-Responder | Pembrolizumab | Squamous | 63F | IIIB | 60% |
| 56 | Non-Responder | Pembrolizumab | Adenocarcinoma | 89F | IV | 50% |
| 57 | Non-Responder | Pembrolizumab | Adenocarcinoma | 59M | IV | 0% |
| | | Chemotherapy | | | | |
| 58 | Non-Responder | Pembrolizumab | Adenocarcinoma | 74M | IV | 70% |
| 59 | Non-Responder | Nivolumab | Adenocarcinoma | 74F | IV | 25% |
| 60 | Non-Responder | Nivolumab | Squamous | 74M | IV | 2% |
| 61 | Non-Responder | Pembrolizumab | Adenocarcinoma | 73F | IV | 50% |
| 62 | Non-Responder | Pembrolizumab | Squamous | 65M | IIIB | <50% |
| 63 | Non-Responder | Pembrolizumab | Adenocarcinoma | 65F | IV | 90% |
| 64 | Non-Responder | Pembrolizumab | Squamous | 68M | IIIB | 50-75% |
| 65 | Non-Responder | Pembrolizumab | Squamous | 68F | IV | 50-70% |
| 66 | Non-Responder | Pembrolizumab | Squamous | 60M | IV | 90% |
| 67 | Non-Responder | Pembrolizumab | Squamous | 53F | IV | 75-100% |
| 68 | Non-Responder | Pembrolizumab | Adenocarcinoma | 59F | IV | >50% |
| 69 | Non-Responder | Pembrolizumab | Adenocarcinoma | 76F | IV | 50% |
| 70 | Non-Responder | Pembrolizumab | Squamous | 76F | IV | 75% |
| 71 | Non-Responder | Pembrolizumab | Squamous | 72M | IV | 10% |
| 72 | Non-Responder | Pembrolizumab | Adenocarcinoma | 51F | IV | 50% |
| 73 | Non-Responder | Pembrolizumab | Squamous | 84M | IIB | >75% |
| 74 | Non-Responder | Pembrolizumab | Squamous | 75F | III | >75% |
| 75 | Non-Responder | Pembrolizumab | Adenocarcinoma | 64M | IV | >50% |
| 76 | Non-Responder | Pembrolizumab | Adenocarcinoma | 79F | III | >50% |
| 77 | Non-Responder | Pembrolizumab | Adenocarcinoma | 76F | IV | 1-49% |
| 78 | Non-Responder | Pembrolizumab | Squamous | 70F | IV | 100% |
| 79 | Non-Responder | Pembrolizumab | Adenocarcinoma | 65F | IV | 80% |
| 80 | Non-Responder | Pembrolizumab | Adenocarcinoma | 81M | IV | 1-49% |
| 81 | Non-Responder | Pembrolizumab | Adenocarcinoma | 59M | IV | >50% |
| 82 | Non-Responder | Nivolumab | Squamous | 57F | IV | >50% |
| 83 | Non-Responder | Pembrolizumab | Adenocarcinoma | 75M | IV | >50% |
| 84 | Non-Responder | Pembrolizumab | Squamous | 79F | IIIA | 50% |
| 85 | Non-Responder | Pembrolizumab | Adenocarcinoma | 71F | IV | 100% |
| 86 | Non-Responder | Pembrolizumab | Adenocarcinoma | 77F | IV | >50% |
| 87 | Non-Responder | Pembrolizumab | Poorly | 50M | IV | <1% |
| | | | differentiated | | | |
| | | | NSCLC, large | | | |
| | | | cell carcinoma | | | |
| | | | favored | | | |
| 88 | Non-Responder | Pembrolizumab | Adenocarcinoma | 66F | IV | <1% |
| 89 | Non-Responder | Pembrolizumab | Adenocarcinoma | 78M | IV | 70% |
| 90 | Non-Responder | Nivolumab | Adenocarcinoma | 78F | IV | 15% |
| 91 | Non-Responder | Pembrolizumab | Squamous | 63F | IV | 10% |

*Response:
Responder: Complete response or partial response
Non-Responder: Stabel disease or progression
NSCLC: nonsmall cell lung cancer

What is claimed is:

1. A method of detecting tumor-derived exosomal (TEX) biomarker in a subject using liquid biopsy, comprising:
obtaining a liquid biopsy from the subject;
capturing TEX in the liquid biopsy using an antibody specific to the TEX biomarker;
detecting surface plasmon resonance (SPR) signal generated by the capture of TEX;
measuring the expression of TEX biomarker based on SPR signal;
analyzing the signal between the sample with TEX biomarker and reference sample, wherein the reference sample is obtained from a healthy subject; and
administering a therapeutically effective dose of a humanized TEX biomarker specific antibody to the subject with a higher TEX biomarker expression compared to the reference sample from a healthy subject.

2. The method of claim 1, wherein the TEX biomarker is an alpha-linked Thomsen-Friedenreich glycoantigen (TF-Ag-α; Galβ1-3GalNAc alpha).

3. The method of claim 1, wherein the antibody specific to a biomarker present on TEX is JAA-F11.

4. The method of claim 1, wherein liquid biopsy comprises cerebrospinal fluid, saliva, amniotic fluid, ascites, blood, breast milk, bone marrow, or urine.

5. The method of claim 1, wherein the biochip further enables multiplex detection of additional TEX biomarkers co-expressed on TEX, wherein the additional TEX biomarkers comprise PD-L1, PD-1, LAG-3, TIM-3, or CTLA-4.

6. The method of claim 1, wherein the expression level of the TF-Ag-α in the sample is calculated using following equation:
$$Expression of TEX TF - Ag - α = {( \frac{{I}_{TEX TF - Ag - α} - {I}_{PBS}}{{I}_{PBS} - {I}_{water}} )}_{JAA - F 11 antibody modified biochip} - {( \frac{{I}_{TEX TF - Ag - α} - {I}_{PBS}}{{I}_{PBS} - {I}_{water}} )}_{IgG 3 control antibody modified biochip}$$
wherein water and PBS emit baseline signal; wherein IgG3 is a control antibody; wherein a higher expression level of TEX TF-Ag-α in the sample relative to the reference sample indicates presence of a cancer.

7. The method of claim 1, wherein the TEX are extracted from subjects with lung cancer, breast cancer, prostate cancer, colon cancer, ovarian cancer, or bladder cancer.

8. A system to detect expression level of a TEX biomarker in a subject using liquid biopsy, comprising:
a biochip for detection of TEX biomarker in a liquid biopsy sample;
an antibody immobilized on the biochip, wherein the antibody is specific to the TEX biomarker;
a biosensor (photodetector) to record an SPR signal from the liquid biopsy sample;
wherein the SPR signal is obtained when TEX biomarker binds to the antibody; and
an equation to analyze expression level of the TEX biomarker, wherein a high TEX biomarker expression compared to a reference sample indicates presence of a cancer.

9. The system to detect expression level of a TEX biomarker of claim 8, wherein the TEX are extracted from subjects with lung cancer, breast cancer, prostate cancer, colon cancer, ovarian cancer, or bladder cancer.

10. The system to detect expression level of a TEX biomarker of claim 8, wherein the liquid biopsy sample comprises cerebrospinal fluid, saliva, amniotic fluid, ascites, blood, breast milk, bone marrow, or urine.

11. The system to detect expression level of a TEX biomarker of claim 8, wherein the TEX biomarker comprises a protein, a nucleic acid, a carbohydrate or a lipid.

12. The system to detect expression level of a TEX biomarker of claim 11, wherein the carbohydrate is an alpha-linked Thomsen-Friedenreich glycoantigen (TF-Ag-α; Galβ1-3GalNAc alpha).

13. The system to detect expression level of a TEX biomarker of claim 8, wherein the TEX biomarker specific antibody is JAA-F11.

14. A method to assess the efficacy of a cancer immunotherapy response in a liquid biopsy comprising:
obtaining a liquid biopsy;
capturing TEX in a liquid biopsy using TEX biomarker specific antibody (first antibody) attached to a biochip;
detecting a TEX biomarker in the sample by measuring SPR related signal using a SPR biosensor;
measuring TEX biomarker expression;
exposing the biochip to a second antibody specific to a biomarker associated with response to an immune checkpoint inhibitor co-expressed on TEX;
measuring an SPEF signal related to a TEX biomarker associated with the response to an immune checkpoint inhibitor (TEX-ICI); and
administering a therapeutically effective dose of an immune checkpoint inhibitor (ICI) to a subject with increased expression of the TEX biomarker relative to a subject with no or decreased expression of TEX.

15. The method of claim 14, wherein the ICI comprises anti-immune checkpoint inhibitor monoclonal antibodies, wherein the monoclonal antibodies comprise anti-PD-1, anti-PD-L1, anti-LAG3, anti-TIM3 or anti-CTLA-4.

16. The method of claim 14, wherein the TEX biomarker is an alpha-linked Thomsen-Friedenreich glycoantigen (TF-Ag-α; Galβ1-3GalNAc alpha).

17. The method of claim 14, wherein the TEX biomarker specific antibody (first antibody) is JAA-F11.

18. The method of claim 14, wherein the biomarker associated with immune checkpoint proteins co-expressed on TEX in the sample is calculated using following equation:
$$Expression of {TEX}^{TF - Ag - α} PD - L 1 = ( \frac{{I}_{after APC - PD - L 1} - {I}_{before APC - PD - L 1}}{{I}_{before APC - PD - L 1}} ) / ( Expression of TEX TF - Ag - α ) = ( \frac{{I}_{after APC - PD - L 1} - {I}_{before APC - PD - L 1}}{{I}_{before APC - PD - L 1}} ) / ( \frac{{I}_{TEX TF - Ag - α} - {I}_{PBS}}{{I}_{PBS} - {I}_{water}} )$$
wherein water and PBS emit baseline signal; wherein TF-Ag-α is a TEX biomarker; wherein TEX TF-Ag-α is the level of TF-Ag-α expressed on TEX; wherein TEX^{TF-Ag-α} is tumor-derived exosomes that express TF-Ag-α; wherein TEX^{TF-Ag-α} PD-L1 is the biomarker associated with immune checkpoint proteins co-expressed on TEX^{TF-Ag-α}; wherein PD-L1 is the biomarker associated with response to an ICI; wherein APC is a fluorescent dye attached to anti-PD-L1 antibody; wherein after APC-PD-L1 is fluorescence intensity of APC after incubating biochip with anti-APC-PD-L1 antibody; and wherein before APC-PD-L1 is fluorescence intensity of APC before incubating biochip with anti-APC-PD-L1 antibody.

19. The method of claim 14, wherein the liquid biopsy comprises cerebrospinal fluid, saliva, amniotic fluid, ascites, blood, breast milk, bone marrow, or urine.
